(19) United States
(12) Patent Application Publication
(10) Pub. No.: US 2026/0335422 A1
(43) Pub. Date: Oct. 29, 2026

(54) MODIFIED STREPTOCOCCAL IMMUNOGEN AND USES THEREOF

(71) Applicant: GPN Vaccines Pty Ltd, Yarralumla, Australian Capital Territory (AU)

(72) Inventors: Chloe Gates, Henley Beach, South Australia (AU); Erin Bridget Brazel, Woodville West, South Australia (AU); Mohammed Alsharifi, Elizabeth Park, South Australia (AU); Timothy Raymond Hirst, Yarralumla, Australian Capital Territory (AU); James Cleland Paton, Parkside, South Austrlia (AU); Eve Victoria Singleton, Goodwood, South Austrlia (AU)

(21) Appl. No.: 19/486,732

(22) PCT Filed: May 22, 2024
(86) PCT No.: PCT/AU2024/050524

§ 371 Date: Nov. 21, 2025

(30) Foreign Application Priority Data
May 22, 2023 (AU) 2023901596

(51) Int. Cl. A61K 39/09; A61K 39/00; A61P 37/04

(52) U.S. Cl. CPC A61K 39/092; A61P 37/04; A61K 2039/521; A61K 2039/522; A61K 2039/54

(57) ABSTRACT

The present disclosure relates to an immunogenic composition comprising an attenuated and/or killed streptococcal bacterial strain comprising a modification that attenuates, reduces or prevents functional expression of LytC (or a homologue thereof). Such a composition may be utilised in a vaccine composition. The immunogenic composition and/or the vaccine composition may be utilised in methods of inducing an immune response to at least one target streptococcal species and/or serotype thereof and/or preventing or reducing an infection and or disease or condition in a subject by at least one target streptococcal species and/or serotype thereof.

### RELATED APPLICATIONS

The present application claims priority from Australian Provisional Application No. 2023901596, filed on 22 May 2023, the contents of which is incorporated by reference herein in its entirety.

### FIELD OF THE DISCLOSURE

The present disclosure relates to an immunogenic composition. In one form, the present disclosure relates to an attenuated or killed streptococcal bacteria strain.

### BACKGROUND OF THE DISCLOSURE

Any discussion of the prior art throughout the specification should in no way be considered as an admission that such prior art is widely known or forms part of the common general knowledge in the field.

Currently used vaccines against streptococcal infection are generally polysaccharide vaccines containing purified capsular polysaccharides from multiple serotypes (for example, the PCV23 vaccine containing polysaccharides from 23 serotypes), or conjugated vaccines containing capsular polysaccharides conjugated to diphtheria toxoid or other protein antigens, including of non-streptococcal or streptococcal origin. These polysaccharide-targeted vaccines elicit strictly serotype-dependent protection. Serotype replacement is a significant problem associated with polysaccharide vaccines, because multivalent conjugate vaccines induce immunity against only a subset of the more than 100 serotypes of Streptococcus pneumoniae recognised to date, most of which have been shown to cause disease. The immunity induced by commercially available streptococcal vaccines is thus inadequate to establish broad immunity against the majority of streptococcal species and/or serotypes within certain pathogenic species. Accordingly, there is a need for an improved streptococcal vaccine.

Streptococcal strains such as Streptococcus pneumoniae may possess autolytic enzymes such as CbpD, LytA, LytB and LytC. LytC is a cell wall hydrolase that has been proposed to contribute to fratricide with LytA by facilitating the lysis of non-competent pneumococcal cells (Eldholm et al., 2009).

The present disclosure relates to an unexpected finding that the use of an attenuated or killed whole cell Streptococcus vaccine from which a lytC gene (or a homologue thereof) has been modified to attenuate, reduce and/or prevent functional expression of LytC (or a homologue thereof) provides improved vaccine immunogenicity.

### SUMMARY OF THE DISCLOSURE

The present disclosure relates to an attenuated or killed streptococcal bacterial strain comprising a modification that attenuates, reduces or prevents functional expression of LytC (or a homologue thereof). Surprisingly, compared with a parental vaccine strain lacking the modification to lytC, mice and rabbits immunised with a composition comprising a streptococcal bacterial strain harbouring a deletion of lytC produced IgG antibodies with enhanced binding to the surface of pneumococcal strains. Further, the antibodies produced by immunisation with strains harbouring a deletion of lytC exhibit higher functional opsonophagocytic responses, a recognised surrogate endpoint of pneumococcal vaccine efficacy. These findings illustrate a novel mechanism not previously recognised for improving the immunogenicity of whole cell vaccines by modifying expression of or deleting the lytC gene. This could not be foreseen based on prior studies.

The present invention relates to at least the following embodiments:

Embodiment 1. An immunogenic composition comprising an attenuated and/or killed streptococcal bacterial strain comprising a modification that attenuates, reduces or prevents functional expression of LytC or a homologue thereof. In some examples, an immunogenic composition may consist of or essentially consist of an attenuated and/or killed streptococcal bacterial strain comprising a modification that attenuates, reduces or prevents functional expression of LytC or a homologue thereof.

Embodiment 2. The immunogenic composition of embodiment 1, wherein the modification comprises a deletion of the lytC gene or a homologue thereof. In some examples, the modification may consist of or essentially consist of a deletion of the lytC gene or a homologue thereof.

Embodiment 3. The immunogenic composition of embodiment 1 or 2, wherein the immunogenic composition is a cross-protective immunogenic composition.

Embodiment 4. The immunogenic composition of any one of embodiments 1 to 3, wherein the attenuated and/or killed streptococcal bacterial strain is derived from a parental strain lacking the modification, and the attenuated and/or killed streptococcal bacterial strain improves effectiveness of an immune response compared to the parental strain.

Embodiment 5. The immunogenic composition of any one of embodiments 1 to 4, wherein the attenuated and/or killed streptococcal bacterial strain is derived from a parental strain lacking the modification, and the attenuated and/or killed streptococcal bacterial strain has improved stability compared to the parent strain.

Embodiment 6. The immunogenic composition of any one of embodiments 1 to 5, wherein the attenuated and/or killed streptococcal bacterial strain further comprises modification or deletion of genes selected from the group consisting of lytA or a homologue thereof, ply or a homologue thereof, and psaA or a homologue thereof.

Embodiment 7. The immunogenic composition of any one of embodiments 1 to 6, wherein the attenuated and/or killed streptococcal bacterial strain further comprises a modification or deletion of a lytA gene. or a homologue thereof.

Embodiment 8. The immunogenic composition of any one of embodiments 1 to 7, wherein the attenuated and/or killed streptococcal bacterial strain further comprises a modification or deletion of a ply gene or a homologue thereof.

Embodiment 9. The immunogenic composition of any one of embodiments 1 to 8, wherein the attenuated and/or killed streptococcal bacterial strain further comprises a toxoid version of Ply designated PdT or a homologue thereof.

Embodiment 10. The immunogenic composition of any one of embodiments 1 to 9, wherein the attenuated and/or killed streptococcal bacterial strain further comprises a modification or deletion of a psaA gene or a homologue thereof.

Embodiment 11. The immunogenic composition of any one of embodiments 1 to 10, wherein the attenuated and/or killed streptococcal bacterial strain further comprises a modification or deletion of a lytA gene or a homologue thereof, a modification or deletion of a ply gene or a homologue thereof and a modification or deletion of a psaA gene or a homologue thereof, and further comprises a toxoid version of Ply designated PdT or a homologue thereof.

Embodiment 12. The immunogenic composition of any one of embodiments 1 to 11, wherein the attenuated and/or killed streptococcal bacterial strain expresses capsular polysaccharide.

Embodiment 13. The immunogenic composition of any one of embodiments 1 to 12, wherein the attenuated and/or killed streptococcal bacterial strain expresses a reduced level of capsular polysaccharide or no capsular polysaccharide.

Embodiment 14. The immunogenic composition of any one of embodiments 1 to 13, wherein the composition comprises a single attenuated and/or killed streptococcal bacterial strain comprising a modification that attenuates, reduces or prevents functional expression of LytC or a homologue thereof.

Embodiment 15. The immunogenic composition of any one of embodiments 1 to 13, wherein the composition comprises a plurality of attenuated and/or killed streptococcal bacterial strains each comprising a modification that attenuates, reduces or prevents functional expression of LytC or a homologue thereof.

Embodiment 16. The immunogenic composition of any one of embodiments 1 to 15, wherein the attenuated and/or killed streptococcal bacterial strain is of a species selected from the group consisting of Streptococcus agalactiae, Streptococcus bovis, Streptococcus canis, Streptococcus dysgalactiae, Streptococcus equi, Streptococcus equinus, Streptococcus equisimilis, Enterococcus faecalis, Enterococcus faecium, Streptococcus iniae, Streptococcus milleri, Streptococcus mutans, Streptococcus pneumoniae, Streptococcus pyogenes, Streptococcus salivarius, Streptococcus sanguinis, Streptococcus suis, and Streptococcus uberis.

Embodiment 17. The immunogenic composition of any one of embodiments 1 to 16, wherein the attenuated and/or killed streptococcal bacterial strain comprises a Streptococcus pneumoniae strain.

Embodiment 18. The immunogenic composition of embodiment 17, wherein the Streptococcus pneumoniae strain is derived from Rx1.

Embodiment 19. The immunogenic composition of any one of embodiments 1 to 18, wherein the attenuated and/or killed streptococcal bacterial strain induces an immune response to an at least one target streptococcal species and/or serotype thereof.

Embodiment 20. The immunogenic composition of any one of embodiments 1 to 19, wherein the attenuated and/or killed streptococcal bacterial strain induces an immune response to a plurality of target streptococcal species and/or serotypes thereof.

Embodiment 21. The immunogenic composition of any one of embodiments 1 to 20, wherein the attenuated and/or killed streptococcal bacterial strain induces a cross-protective immune response to the at least one target streptococcal species and/or serotype thereof.

Embodiment 22. The immunogenic composition of any one of embodiments 1 to 21, wherein the attenuated and/or killed streptococcal bacterial strain induces a cross-protective immune response to a plurality of target streptococcal species and/or serotypes thereof.

Embodiment 23. The immunogenic composition of any one of embodiments 1 to 20, wherein the attenuated and/or killed streptococcal bacterial strain is derived from the same strain, serotype or species as the at least one target streptococcal species and/or serotype thereof.

Embodiment 24. The immunogenic composition of any one of embodiments 1 to 22, wherein the attenuated and/or killed streptococcal bacterial strain is derived from a different strain, serotype or species as the at least one target streptococcal species and/or serotype thereof.

Embodiment 25. The immunogenic composition of any one of embodiments 1 to 24, wherein the at least one target streptococcal bacterial species and/or serotype thereof selected from the group consisting of: Streptococcus agalactiae, Streptococcus bovis, Streptococcus canis, Streptococcus dysgalactiae, Streptococcus equi, Streptococcus equinus, Streptococcus equisimilis, Enterococcus faecalis, Enterococcus faecium, Streptococcus iniae, Streptococcus milleri, Streptococcus mutans, Streptococcus pneumoniae, Streptococcus pyogenes, Streptococcus salivarius, Streptococcus sanguinis, Streptococcus suis and Streptococcus uberis.

Embodiment 26. The immunogenic composition of any one of embodiments 1 to 25, wherein the immunogenic composition is a killed whole cell immunogenic composition.

Embodiment 27. The immunogenic composition of any one of embodiments 1 to 26, wherein the killed whole cell immunogenic composition is selected from the group consisting of a chemical treatment killed whole cell immunogenic composition, a thermal treatment killed whole cell immunogenic composition, an irradiation killed whole cell immunogenic composition, a high hydrostatic pressure killed whole cell immunogenic composition, a pulsed electric field killed whole cell immunogenic composition, an ultrashort pulsed laser killed whole cell immunogenic composition, an ultrasound under pressure killed whole cell immunogenic composition, UV-irradiation, and a microbial inactivation killed whole cell immunogenic composition.

Embodiment 28. The immunogenic composition of any one of embodiments 1 to 27, wherein the killed whole cell immunogenic composition is a photon-irradiation killed whole cell immunogenic composition.

Embodiment 29. The immunogenic composition of any one of embodiments 1 to 28, wherein the killed whole cell immunogenic composition is a gamma-irradiation killed whole cell immunogenic composition.

Embodiment 30. The immunogenic composition of any one of embodiments 1 to 28, wherein the killed whole cell immunogenic composition is an X-ray-irradiation killed whole cell immunogenic composition.

Embodiment 31. The immunogenic composition of any one of embodiments 1 to 31, wherein the killed whole cell immunogenic composition is a gamma-irradiated and X-ray-irradiation killed whole cell immunogenic composition.

Embodiment 32. The immunogenic composition of any one of embodiments 1 to 25, wherein the immunogenic composition is an attenuated whole cell immunogenic composition.

Embodiment 33. The immunogenic composition of any one of embodiments 1 to 32, further comprising at least one of an adjuvant, a pharmaceutically acceptable excipient, and a pharmaceutically acceptable carrier.

Embodiment 34. The immunogenic composition of any one of embodiments 1 to 33, wherein the composition does not comprise an adjuvant.

Embodiment 35. A vaccine composition comprising the immunogenic composition of any one of embodiments 1 to 34.

Embodiment 36. Use of the immunogenic composition of any one of embodiments 1 to 34 or the vaccine composition of embodiment 35 as an immunogen.

Embodiment 37. Use of the immunogenic composition of any one of embodiments 1 to 34 or the vaccine composition of embodiment 35 as a vaccine.

Embodiment 38. A method of inducing an immune response in a subject to at least one target streptococcal species and/or serotype thereof, the method comprising administering a therapeutically effective amount of the immunogenic composition of any one of embodiments 1 to 34 or the vaccine composition of embodiment 35 to the subject.

Embodiment 39. A method of inducing a cross-protective immune response in a subject to at least one target streptococcal species and/or serotype thereof, the method comprising administering a therapeutically effective amount of the immunogenic composition of any one of embodiments 1 to 34 or the vaccine composition of embodiment 35 to the subject.

Embodiment 40. A method of preventing or reducing an infection in a subject by at least one target streptococcal species and/or serotype thereof, the method comprising administering to a subject a therapeutically effective amount of the immunogenic composition of any one of embodiments 1 to 34 or the vaccine composition of embodiment 35 to the subject.

Embodiment 41. A method of preventing or reducing a disease or condition in a subject caused by an at least one target streptococcal species and/or serotype thereof, the method comprising administering to a subject a therapeutically effective amount of the immunogenic composition of any one of embodiments 1 to 34 or the vaccine composition of embodiment 35 to the subject.

Embodiment 42. The method of any one of embodiments 38 to 42 wherein the attenuated and/or killed streptococcal bacterial strain is derived from the same strain, serotype or species as the at least one target streptococcal species and/or serotype thereof.

Embodiment 43. The method of any one of embodiments 38 to 42 wherein the attenuated and/or killed streptococcal bacterial strain is derived from a different strain, serotype or species as the at least one target streptococcal species and/or serotype thereof.

Embodiment 44. The method of any one of embodiments 38 to 42 wherein the at least one target streptococcal species and/or serotype thereof comprises a plurality of target streptococcal species and/or serotypes thereof.

Embodiment 45. The method of any one of embodiments 38 to 42 wherein the target streptococcal species and/or serotype thereof comprises a streptococcal bacterial species and/or serotype thereof selected from the group consisting of: Streptococcus agalactiae, Streptococcus bovis, Streptococcus canis, Streptococcus dysgalactiae, Streptococcus equi, Streptococcus equinus, Streptococcus equisimilis, Enterococcus faecalis, Enterococcus faecium, Streptococcus iniae, Streptococcus milleri, Streptococcus mutans, Streptococcus pneumoniae, Streptococcus pyogenes, Streptococcus salivarius, Streptococcus sanguinis, Streptococcus suis and Streptococcus uberis.

Embodiment 46. The method of any one of embodiments 38 to 42 wherein the immunogenic composition or the vaccine composition is administered to the subject by an administration route selected from the group consisting of intranasal, intravenous, intramuscular, subcutaneous, oral, intraocular, transmucosal, and transdermal administration.

Embodiment 47. The method of embodiment 41, wherein the disease or condition is selected from the group consisting of a respiratory tract infection, pneumonia, ear infection, earache, middle ear infection, otitis media, sinusitis, meningitis, conjunctivitis, bacteraemia, septicaemia, a joint infection, a bone infection, septic arthritis, osteomyelitis, a soft tissue infection, cellulitis, myositis, periorbital cellulitis, an abscess, peritonitis, a cardiac infection, endocarditis, and pericarditis.

Embodiment 48. A method of preparing the immunogenic composition of any one of embodiments 1 to 34, or the vaccine composition of embodiment 35, the method comprising:
(i) photon-irradiating a preparation of the streptococcal bacterial strain comprising a modification that attenuates, reduces or prevents functional expression of LytC or a homologue thereof to thereby kill or physically attenuate the bacteria to produce photon-irradiated streptococcal bacteria; and
(ii) combining the photon-irradiated streptococcal bacteria with a pharmaceutically acceptable excipient, diluent and/or carrier, and optionally, an adjuvant.

Embodiment 49. The method of embodiment 48, wherein the photon-irradiating comprises exposing the streptococcal bacterial strain to gamma-radiation.

Embodiment 50. The method of embodiment 49, wherein the photon-irradiating comprises exposing the streptococcal bacterial strain to X-radiation.

Embodiment 51. The method of any one of embodiments 48 to 50, wherein said photon-irradiating comprises exposing the streptococcal bacterial strain to gamma-radiation and X-radiation.

Embodiment 52. The method of any one of embodiments 48 to 51, wherein the photon irradiating comprises exposing the streptococcal bacterial strain to photon-radiation at energies of at least 0.01 MeV.

Embodiment 53. Use of a therapeutically effective amount of the immunogenic composition of any one of embodiments 1 to 34 or the vaccine composition of embodiment 35 in the preparation of a medicament for inducing an immune response in a subject to at least one target streptococcal species and/or serotype thereof.

Embodiment 54. Use of a therapeutically effective amount of the immunogenic composition of any one of embodiments 1 to 34 or the vaccine composition of embodiment 35 in the preparation of a medicament for inducing a cross-protective immune response in a subject to at least one target streptococcal species and/or serotype thereof.

Embodiment 55. Use of a therapeutically effective amount of the immunogenic composition of any one of embodiments 1 to 34 or the vaccine composition of embodiment 35 in the preparation of a medicament for preventing or reducing an infection in a subject by at least one target streptococcal species and/or serotype thereof.

Embodiment 56. Use of a therapeutically effective amount of the immunogenic composition of any one of embodiments 1 to 34 or the vaccine composition of embodiment 35 in the preparation of a medicament for preventing or reducing a disease or condition in a subject caused by an at least one target streptococcal species and/or serotype thereof.

Embodiment 57. Use of any one of embodiments 53 to 56 wherein the attenuated and/or killed streptococcal bacterial strain is derived from the same strain, serotype or species as the at least one target streptococcal species and/or serotype thereof.

Embodiment 58. Use of any one of embodiments 53 to 56 wherein the attenuated and/or killed streptococcal bacterial strain is derived from a different strain, serotype or species as the at least one target streptococcal species and/or serotype thereof.

Embodiment 59. Use of any one of embodiments 53 to 56 wherein the at least one target streptococcal species and/or serotype thereof comprises a plurality of target streptococcal species and/or serotypes thereof.

Embodiment 60. Use of any one of embodiments 53 to 56 wherein the target streptococcal species and/or serotype thereof comprises a streptococcal bacterial species and/or serotype thereof selected from the group consisting of: Streptococcus agalactiae, Streptococcus bovis, Streptococcus canis, Streptococcus dysgalactiae, Streptococcus equi, Streptococcus equinus, Streptococcus equisimilis, Enterococcus faecalis, Enterococcus faecium, Streptococcus iniae, Streptococcus milleri, Streptococcus mutans, Streptococcus pneumoniae, Streptococcus pyogenes, Streptococcus salivarius, Streptococcus sanguinis, Streptococcus suis and Streptococcus uberis.

Embodiment 61. Use of any one of embodiments 53 to 56 wherein the immunogenic composition or the vaccine composition is administered to the subject by an administration route selected from the group consisting of intranasal, intravenous, intramuscular, subcutaneous, oral, intraocular, transmucosal, and transdermal administration.

Embodiment 62. Use of embodiment 56, wherein the disease or condition is selected from the group consisting of a respiratory tract infection, pneumonia, ear infection, earache, middle ear infection, otitis media, sinusitis, meningitis, conjunctivitis, bacteraemia, septicaemia, a joint infection, a bone infection, septic arthritis, osteomyelitis, a soft tissue infection, cellulitis, myositis, periorbital cellulitis, an abscess, peritonitis, a cardiac infection, endocarditis, and pericarditis.

Embodiment 63. A therapeutically effective amount of the immunogenic composition of any one of embodiments 1 to 34 or the vaccine composition of embodiment 35 for use in inducing an immune response in a subject to at least one target streptococcal species and/or serotype thereof.

Embodiment 64. A therapeutically effective amount of the immunogenic composition of any one of embodiments 1 to 34 or the vaccine composition of embodiment 35 for use in inducing a cross-protective immune response in a subject to at least one target streptococcal species and/or serotype thereof.

Embodiment 65. A therapeutically effective amount of the immunogenic composition of any one of embodiments 1 to 34 or the vaccine composition of embodiment 35 for use in preventing or reducing an infection in a subject by at least one target streptococcal species and/or serotype thereof.

Embodiment 66. A therapeutically effective amount of the immunogenic composition of any one of embodiments 1 to 34 or the vaccine composition of embodiment 35 for use in preventing or reducing a disease or condition in a subject caused by an at least one target streptococcal species and/or serotype thereof, the method comprising administering to a subject a therapeutically effective amount of the immunogenic composition of any one of embodiments 1 to 34 or the vaccine composition of embodiment 35 to the subject.

Embodiment 67. The composition of any one of embodiments 63 to 66 wherein the attenuated and/or killed streptococcal bacterial strain is derived from the same strain, serotype or species as the at least one target streptococcal species and/or serotype thereof.

Embodiment 68. The composition of any one of embodiments 63 to 66 wherein the attenuated and/or killed streptococcal bacterial strain is derived from a different strain, serotype or species as the at least one target streptococcal species and/or serotype thereof.

Embodiment 69. The composition of any one of embodiments 63 to 66 wherein the at least one target streptococcal species and/or serotype thereof comprises a plurality of target streptococcal species and/or serotypes thereof.

Embodiment 70. The composition of any one of embodiments 63 to 66 wherein the target streptococcal species and/or serotype thereof comprises a streptococcal bacterial species and/or serotype thereof selected from the group consisting of: Streptococcus agalactiae, Streptococcus bovis, Streptococcus canis, Streptococcus dysgalactiae, Streptococcus equi, Streptococcus equinus, Streptococcus equisimilis, Enterococcus faecalis, Enterococcus faecium, Streptococcus iniae, Streptococcus milleri, Streptococcus mutans, Streptococcus pneumoniae, Streptococcus pyogenes, Streptococcus salivarius, Streptococcus sanguinis, Streptococcus suis and Streptococcus uberis.

Embodiment 71. The composition of any one of embodiments 63 to 66 wherein the immunogenic composition or the vaccine composition is administered to the subject by an administration route selected from the group consisting of intranasal, intravenous, intramuscular, subcutaneous, oral, intraocular, transmucosal, and transdermal administration.

Embodiment 72. The composition of embodiment 66, wherein the disease or condition is selected from the group consisting of a respiratory tract infection, pneumonia, ear infection, earache, middle ear infection, otitis media, sinusitis, meningitis, conjunctivitis, bacteraemia, septicaemia, a joint infection, a bone infection, septic arthritis, osteomyelitis, a soft tissue infection, cellulitis, myositis, periorbital cellulitis, an abscess, peritonitis, a cardiac infection, endocarditis, and pericarditis.

### BRIEF DESCRIPTION OF DRAWINGS

FIG. 1 provides a schematic diagram illustrating the generation of GPN-002, GPN-002ΔlytC::KanR, GPN-003 and GPN-001ΔlytC::KanR from GPN-001 derivatives of S. pneumoniae Rx1.

FIG. 2 provides a photographic image of an electrophoresis gel showing PCR products confirming the incorporation of kanamycin resistance cassette at the lytC region of GPN-002.

FIG. 3 provides a graphical representation of the optical density (OD₆₀₀) of washed GPN-002 and GPN-002ΔlytC::kanR bacteria suspended in PBS with 20% glycerol over time at 25° C.

FIG. 4 provides a graphical representation of the optical density (OD₆₀₀) of washed GPN-002 and GPN-002ΔlytC::kanR bacteria suspended in PBS with 20% glycerol over time at 25° C.

FIG. 5 provides a photographic image of an electrophoresis gel showing PCR products confirming the absence of the lytA, ply/PdT substitution, absence of psaA, and absence of lytC in GPN-003.

FIG. 6 provides (upper panel) the DNA sequence and protein translation of the ply region of Rx1; and (lower panel) the DNA sequence and protein translation of the pdt region of GPN-003.

FIG. 7 provides a graphical representation of the optical density (OD₆₀₀) of washed GPN-002 and GPN-003 bacteria suspended in PBS with 20% glycerol over time at 25° C.

FIG. 8 provides graphical representation of antibody responses in mice immunised with PBS (mock control), Gamma-PN2 and Gamma-PN3 by ELISA using Rx1 as capture antigen showing (A) total pneumococcal-specific IgG responses at absorbance (450/620 nm); (B) total IgG response as titres; (C) IgG1; (D) IgG2a; (E) IgG2b; (F) IgG3. Data are presented as mean absorbance (450/620 nm)±SEM from individual or pooled serum samples (n=5 mice per group).

FIG. 9 provides graphical representation of IgG raised following mock vaccination or vaccination with Gamma-PN2 or Gamma-PN3 with (A) percentage IgG binding to S. pneumoniae serotype 2 strain D39, (B) geometric mean of fluorescence intensity (GMFI) of IgG binding to S. pneumoniae serotype 2 strain D39, (C) percentage IgG binding to S. pneumoniae serotype 6A strain P9, (D) GMFI of IgG binding to S. pneumoniae serotype 6A strain P9. Data were analysed by one-way ANOVA (** p<0.01, **** p<0.0001).

FIG. 10 provides graphical representation of IgG titres of (A) individual rabbits pre-immunisation and post-third immunisation, and (B) as mean IgG titres (±S.E.M.) within each vaccine group. Data were analysed by one-way ANOVA, nd=not detected.

FIG. 11 provides graphical representation of flow cytometry analysis of rabbit IgG binding to S. pneumoniae serotype 7C bacterial cells following incubation with rabbit serum following vaccination with either Gamma-PN2 or GammaPN3, with (A) the relative fluorescence, (B) the percentage of serotype 7C cells that are bound by rabbit IgG antibodies, and (C) the geometric mean fluorescent intensity of IgG antibodies bound to serotype 7C cells, indicative of the magnitude of IgG binding. Data were analysed by one-way ANOVA (* p<0.05, ** p<0.01, *** p<0.001, **** p<0.0001, ns not significant).

FIG. 12 provides a graphical representation of mean prsA transcription level relative to gyrA transcription level as determined by quantitative reverse transcription polymerase chain reaction (qRT-PCR) in GPN-002 and GPN-003.

FIG. 13 provides a graphical representation showing (A) survival and (B) weight in grams of mice inoculated with either S. pneumoniae serotype 2 strain D39 at 10⁴ CFU/mouse; or GPN-002 or GPN-003 at a 10,000-fold higher dose (10⁸ CFU/mouse).

FIG. 14 provides a schematic diagram illustrating the generation of GPN-002, GPN-002ΔlytC::KanR, GPN-003 and GPN-001ΔlytC::KanR and GPN 001ΔlytC from GPN-001 derivatives of S. pneumoniae Rx1.

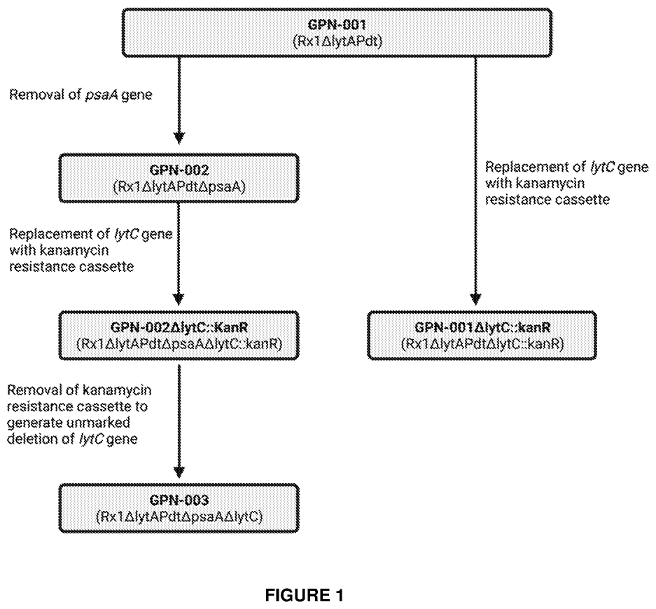

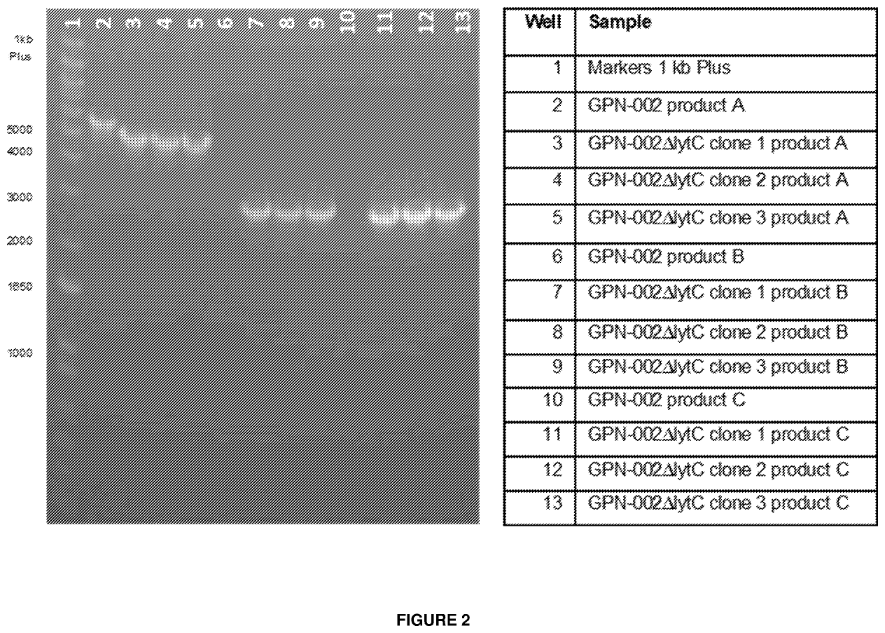

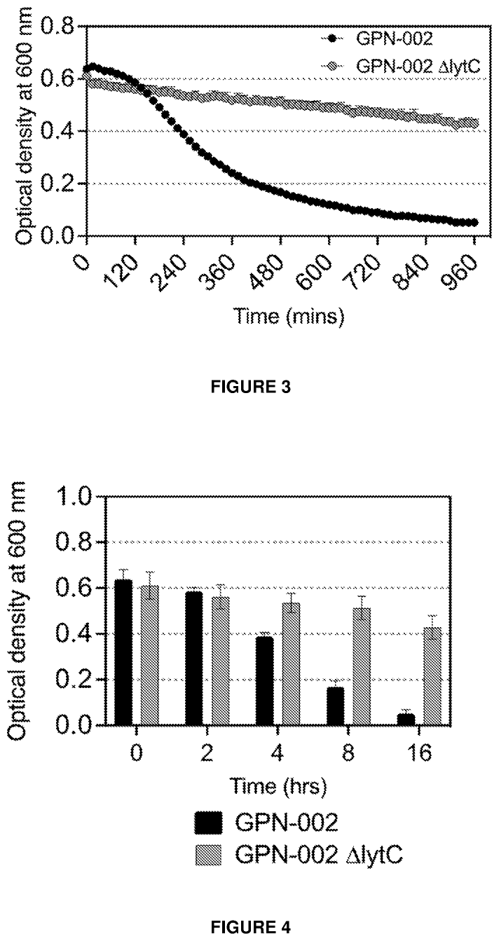

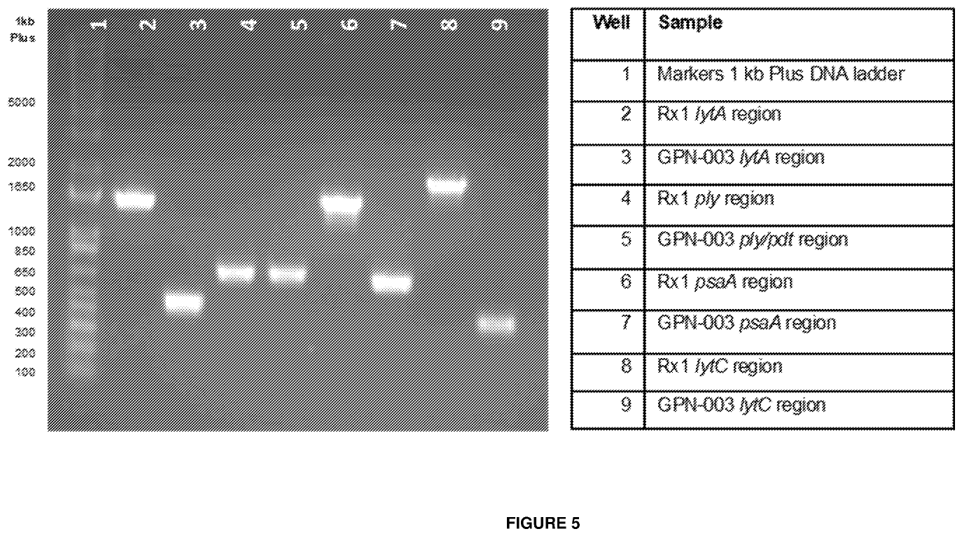

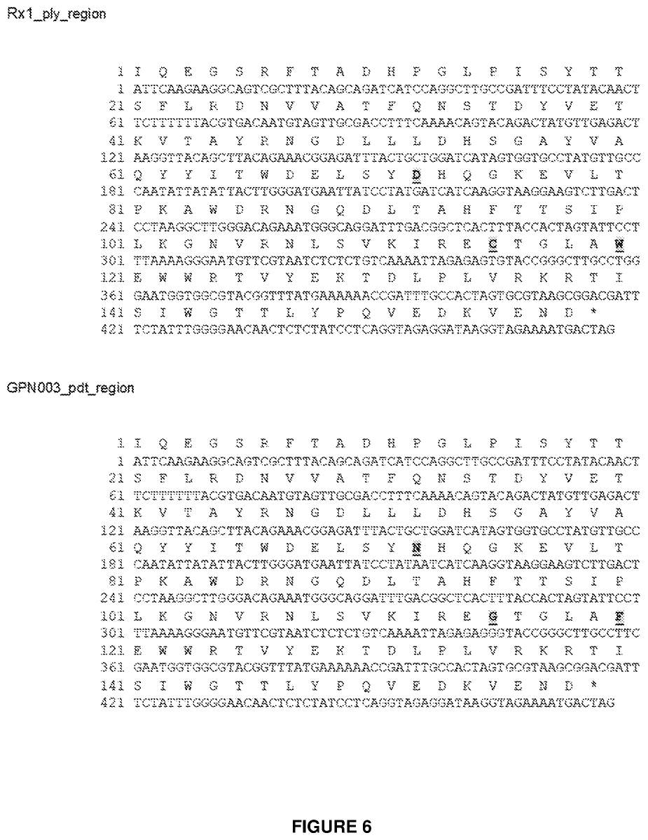

### DEFINITIONS

In the context of this specification, the terms “a” and “an” are used herein to refer to one or to more than one (i.e., to at least one) of the grammatical object of the article. By way of example, “an element” means one element or more than one element.

The term “about”, “approximately” and “substantially”, as used herein, when applied to a value of interest, refers to a value that is similar to a stated value. In certain embodiments, it is understood to refer to a range of +/−10%, preferably +/−9%, +/−8%, +/−7%, +/−6%, +/−5%, +/−4%, +/−3%, +/−2%, or +/−1%; or +/−0.05% or +/−0.1% of the stated value unless otherwise stated or otherwise evident from the context.

As used herein, the term “attenuated” in the context of bacteria will be understood to mean that the bacteria do not cause significant pathological damage to a host during infection. For example, an attenuated bacteria may only be capable of causing a non-pathogenic infection in a host to which they are administered, or otherwise, may not be capable of establishing infection in a host, or may establish an infection in a host that causes minor or acceptable symptoms. It is to be understood that an attenuated immunogenic composition remains within the host for a time period sufficient to induce an immune response in the host. The bacteria are not however capable of establishing long-term infection or establishing pathogenic infection that is detrimental to a non-immunocompromised host to which the attenuated bacteria are administered.

It will be understood that use of the term “between” herein when referring to a range of numerical values encompasses the numerical values at each endpoint of the range. For example, a polypeptide of between 10 residues and 20 residues in length is inclusive of a polypeptide of 10 residues in length and a polypeptide of 20 residues in length.

The terms “comprise”, “comprises”, “comprised” or “comprising”, “including” or “having” and the like in the present specification and claims are used in an inclusive sense, i.e., to specify the presence of the stated features but not preclude the presence of additional or further features.

As used herein, the term “complementary” is used to describe the relationship between a first nucleotide sequence and a second nucleotide sequence by the base-pairing rules, wherein adenine (A) bases pair with uracil (U) bases in an RNA molecule or thymine (T) bases in a DNA molecule; and cytosine (C) bases pair with guanine (G) bases in both RNA and DNA molecules. For example, for a DNA polynucleotide molecule, the sequence “5′-A-G-T-C-3′” is perfectly complementary to the sequence “3′-T-C-A-G-5′”; noting that in an RNA sequence, uracil (U) is typically used in place of thymine (T).

The degree of complementarity between nucleic acid strands has significant effects on the efficiency and strength of hybridization between nucleic acid strands. This is of particular importance in amplification or detection methods that depend upon binding between nucleic acids. The sequence of a nucleic acid need not be “perfectly” (100%) complementary to that of its target in order to hybridise. Complementarity may be “partial” in which only some of the nucleic acid bases are matched according to the base pairing rules. It will be understood that where the two sequences are “specifically complementary” when the two molecules can hybridise under appropriate conditions, that is, a first polynucleotide molecule comprising a first nucleotide sequence is specifically complementary to a second polynucleotide molecule comprising a second nucleotide sequence, when the two molecules can hybridise and form a duplex structure under conditions appropriate for the reaction being undertaken (for example, ligation, PCR, sequencing, etc). The term “specifically complementary” can be used interchangeably with “substantially complementary”. It is also to be understood that two nucleotide molecules need not be complementary over their entire length. For example, a portion of a first polynucleotide molecule may be specifically complementary and hybridise with a portion of a second polynucleotide molecule. In this example, the two molecules may not be hybridised over portions that are not specifically complementary. These terms may also be used in reference to individual nucleotides, especially within the context of oligonucleotides.

For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not necessarily excluded from the claim as a whole.

The transitional phrase “consisting essentially of” may be used to describe a composition, process or method that includes materials, steps, features, components, or elements, in addition to those literally disclosed, provided that these additional materials, steps, features, components, or elements do not materially affect the basic and novel characteristic(s) of the claimed invention.

The term “cross-protective” when used with reference to a composition of the present disclosure refers to a composition that is capable of inducing a cross-protective immune response.

The term “cross-protective immune response” as used herein refers to an immune response that is induced by a particular antigen (that is, a streptococcal bacterial strain) that is protective against a different target bacterial strain or species as compared to the strain from which the antigen is derived. For example, the immune response induced by a S. pneumoniae serotype 2 strain is cross protective if it protects a subject from infection or lessens disease caused by a different S. pneumoniae serotype, such as serotype 6. Similarly, the immune response induced by a S. pneumoniae serotype 2 strain is cross protective if it protects a subject from infection or lessens disease caused by a different streptococcal species, such as Streptococcus pyogenes.

The term “derivative” or “derived from” as used herein with respect to a bacterial strain is used to refer to a progeny bacterial strain having an at least one modification as compared to a parental strain. The modification may be natural or it may be purposefully introduced. It is to be understood that the parental strain may be an immediate parent (for example, after the introduction of a single modification). Alternatively, the parental strain may be a more distant parent following the introduction of a series of modifications. For example, the GPN-001, GPN-002 and GPN-003 described herein are all considered to be derivatives of S. pneumoniae Rx1; GPN-002 and GPN-003 are considered to be derivatives of GPN-001; and GPN-003 is considered to be a derivative of GPN-001 and GPN-002.

As used herein, the term “effectively” when used with reference to a particular parameter or particular outcome is intended to refer to a sufficient percentage of the parameter or outcome so as to achieve the desired result.

As used herein, “expression” or “expresses” in context of a nucleic acid sequence or protein, refers to translation of an mRNA into a polypeptide, assembly of multiple polypeptides into an intact protein (e.g., enzyme) and/or post-translational modification of a polypeptide or fully assembled protein (e.g., enzyme). In this application, the terms “expression” and “production,” and grammatical equivalents, are used interchangeably.

As used herein, “expresses” in the context of a particular bacterial characteristic or phenotype refers to the presence of the characteristic or phenotype.

As used herein a “homologue” of a protein refers to a protein within a group of proteins that perform the same biological function, and which are expressed by homologous genes. Homologous genes are genes which encode proteins with the same or similar biological function to the protein encoded by the second gene. Homologous genes and nucleic acid sequences can be present in the same or a different organism. Homologous genes include orthologs (i.e., genes expressed in different species that evolved from common ancestral genes by speciation and encode proteins retain the same function), but do not include paralogs (i.e., genes that are related by duplication but have evolved to encode proteins with different functions). Homologous genes include naturally occurring alleles and artificially created variants. Degeneracy of the genetic code provides the possibility to substitute at least one nucleotide of the protein-encoding sequence of a gene or coding sequence with a different base without altering the amino acid sequence of the polypeptide produced from the gene. When optimally aligned, homologous proteins and nucleotide sequences (e.g., genes) of the present invention may, for example, have at least about 20%, 30%, 40%, 50%, 60%, 70%, 75%, 80%, 85%, 90%, 95%, 96%, 97%, 98% or 99% sequence identity over the full length of the reference gene or protein. In an embodiment, a homologous nucleotides sequences (e.g., genes) or homologous protein sequence of the present invention has at least about 20%, 30%, 40%, 50%, 60%, 70%, 75%, 80%, 85%, 90%, 95%, 96%, 97%, 98% or 99% sequence identity to a reference consensus nucleotide or protein sequence over the full length of the reference sequence. A homologous nucleic acid sequence, gene, or protein described herein may be, for example, a homologous nucleic acid sequence, gene, or protein from other bacteria, from other firmicutes, from other bacilli, from other lactobacillales, from there streptococcaceae, and/or from other streptococci.

Genes/nucleic acid sequences and proteins that are homologues of reference sequences can be identified by comparison of homologous amino acid or nucleotide sequence (e.g., manually or using a computer-based tool employing known homology-based search algorithms such as FASTA, BLAST, and Smith-Waterman). A local sequence alignment program (e.g., BLAST), can be used to find similar sequences by searching in a database of sequences, and the summary Expectation value (E-value) can be used to measure the sequence base similarity. As a given sequence hit with the best E-value for a particular organism may not necessarily be an ortholog (i.e., have the same function/encode a protein having the same function), or be the only ortholog, a reciprocal query may be used to filter hit sequences with significant E-values for ortholog identification. The reciprocal query may entail searches of the significant hits against a database of sequences from the base organism that are similar to the sequence of the query sequence. A hit can be identified as an ortholog, when the reciprocal query's best hit is the query sequence itself or a protein encoded by a duplicated gene after speciation.

As used herein, “% sequence identity” means the extent to which two optimally aligned DNA or protein segments are invariant throughout a window of alignment of components, for example nucleotide sequence or amino acid sequence. An “identity fraction” for aligned segments of a test sequence and a reference sequence is the number of identical components that are shared by sequences of the two aligned segments divided by the total number of sequence components in the reference segment over a window of alignment which is the smaller of the full test sequence or the full reference sequence. “% sequence identity” is the identity fraction times 100. Such optimal alignment is understood to be deemed as local alignment of DNA sequences. For protein alignment, a local alignment of protein sequences should allow introduction of gaps to achieve optimal alignment. Percent sequence identity is calculated over the aligned length not including the gaps introduced by the alignment per se.

As used herein the terms “induce”, “inducing”, “enhance” and “enhancing” in the context of immunity or an immune response refer to an increase in immunity or an immune response above existing levels which may be absent or measurable.

As used herein, the term “improved effectiveness” in the context of immunity or an immune response induced by a composition of the disclosure refers to a shift in the immune response that increases protection from infection or disease compared to a base point such as the immune response induced by a parental strain. The shift in immunity may, for example, refer to an increase in a particular subtype of immunoglobulin (e.g., IgG1, IgG2a, IgG2b, IgG3, IgG4, etc, IgA, IgE, IgM etc), or a shift in the Th1/Th2 bias, etc.

As used herein, the term “immunogenic composition” refers to a composition or formulation that generates an immune response when administered to a subject.

As used herein, the term “vaccine composition” refers to a composition or formulation that generates an immune response when administered to a subject that is capable of protecting the subject from infection with a pathogenic agent, or alternatively, reducing disease caused by a pathogenic agent.

The terms “immune response” and “immunity” are interchangeably used herein, and refer to subject's reaction to foreign antigens. An immune response may include the induction of a humoral (i.e., B cell) and/or cellular (i.e., T cell) response. Suitably, a humoral immune response may be assessed by measuring the antigen-specific antibodies present in serum of immunized animals in response to introduction of the antigen into the host. The immune response may be assessed by various means, for example, the humoral response can be assessed by enzyme linked immunosorbant assay (ELISA) of sera of immunised mammals, by FACS analysis of antibodies binding to cell surface antigen, by opsonisation assays, by microneutralisation assay of immunized animal sera, etc. Alternatively, or additionally, the cellular response can be measured by FACS analysis, a cytotoxic T cell (CTL) to measure the T cell response from lymphocytes isolated from the spleen or other organs of immunized animals, etc.

The term “isolated” as used herein refers to material that is substantially or essentially free from components that normally accompany it in its native state. For example, an “isolated polynucleotide” as used herein refers to a polynucleotide which has been purified from the sequences which flank it in a naturally occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell, i.e., it is not associated with in vivo substances.

As used herein, the term “labelling” is used to describe the binding, ligation or inclusion of a biological tag to a biological molecule, where the biological tag can be utilised to detect the presence of or quantify the absolute or relative amount of the biological molecule.

The term “nucleotide” as used herein, in its broadest sense, refers to any compound and/or substance that is or can be incorporated into a polynucleotide chain. In an embodiment, a nucleotide is a compound and/or substance that is or can be incorporated into a polynucleotide chain via a phosphodiester linkage. In an embodiment, “nucleotide” refers to individual nucleic acid residues (e.g., nucleotides and/or nucleosides).

The term “nucleotide” can be used interchangeably with “nucleic acid”. In an embodiment, “nucleotide” encompasses RNA as well as single and/or double-stranded DNA and/or cDNA.

The term “messenger RNA” or “mRNA” as used herein refers to an RNA polynucleotide molecule that encodes at least one polypeptide. mRNA as used herein encompasses both modified and unmodified RNA. mRNA may contain one or more coding and non-coding regions.

The phrase “modification that attenuates, reduces or prevents functional LytC expression” as used herein refers to a mutation in or deletion of the lytC gene, which reduces or eliminates the expression of functional LytC protein, meaning that the LytC has reduced or eliminated function.

The term “operably connected” or “operably linked” as used herein refers to the functional relationship between two or more nucleic acid segments such as a gene and a regulatory element including but not limited to a promoter, which then regulates the expression of the gene.

The term “pharmaceutically acceptable” as used herein refers to substances that do not cause substantial adverse allergic or immunological reactions when administered to a subject. A “pharmaceutically acceptable carrier” includes, but is not limited to, solvents, coatings, dispersion agents, wetting agents, isotonic and absorption delaying agents and disintegrants.

As used herein the term “photon-radiation” will be understood to encompass both gamma-radiation (i.e., gamma-rays) and X-radiation (i.e., X-rays). Accordingly, a “photon-irradiated” material may be one that has been exposed to gamma-radiation and which has consequently become “gamma-irradiated”, one that has been exposed to X-radiation and which has consequently become “X-irradiated”, or both. By way of non-limiting example only, to become photon-irradiated a material may be subjected to photon-radiation at energies of at least 0.01 MeV, at least 0.1 MeV, at least 0.5 MeV, between 0.01 MeV and 0.5 MeV, between 0.01 MeV and 1 MeV, between 0.01 MeV and 10 MeV, between 0.5 MeV and 20 MeV, between 0.5 MeV and 15 MeV, between 0.5 MeV and 10 MeV, between 0.5 MeV and 5 MeV, between 0.5 MeV and 2 MeV, or between 1 MeV and 2 MeV (e.g., 1.25 MeV).

As used herein the term “plurality” means more than one. In certain specific aspects or embodiments, a plurality may mean 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or more, and any integer derivable therein, and any range derivable therein.

As used herein, the term “polynucleotide molecule” refers to a DNA or RNA nucleic acid molecule comprising a chain of nucleotides and may include an oligonucleotide molecule or a nucleic acid of interest. A polynucleotide molecule is encoded by a “polynucleotide sequence”, which may also be referred to as a nucleic acid sequence herein. It is understood by those skilled in the art that a nucleotide is typically composed of three distinctive chemical sub-units: a five-carbon sugar molecule (pentose-sugar-ring, deoxyribose in DNA or ribose in RNA), a nucleobase (i.e., adenine (A), cytosine (C), guanine (G), thymine (T) or uracil (U)), and a phosphate group. Chemical convention names the carbon atoms in the sugar molecule from 1′ to 5′, and this convention also dictates that the polynucleotide molecule has a 5′ end and a 3′ end. In a polynucleotide molecule, the 3′ carbon of a first nucleotide is linked to the 5′ carbon of the next nucleotide. Those skilled in the art will understand that polynucleotide sequences are read in a 5′ to 3′ direction, unless specifically stated otherwise.

The term “polynucleotide variant” refers to polynucleotides displaying substantial sequence identity with a reference polynucleotide sequence or polynucleotides that hybridize with a reference sequence under stringent conditions. The term also encompasses polynucleotides that are distinguished from a reference polynucleotide by the addition, deletion or substitution of at least one nucleotide. Accordingly, the term “polynucleotide variant” includes polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. The term “polynucleotide variant” also includes naturally occurring allelic variants. The terms “peptide variant” and “polypeptide variant” and the like refer to peptides and polypeptides that are distinguished from a reference peptide or polypeptide by the addition, deletion or substitution of at least one amino acid residue. In certain examples, a peptide or polypeptide variant is distinguished from a reference peptide or polypeptide by one or more substitutions, which may be conservative or non-conservative. In certain examples, the peptide or polypeptide variant comprises conservative substitutions and, in this regard, it is well understood in the art that some amino acids may be changed to others with broadly similar properties without changing the nature of the activity of the peptide or polypeptide. Peptide and polypeptide variants also encompass peptides and polypeptides in which one or more amino acids have been added or deleted, or replaced with different amino acids.

As used herein, an “oligonucleotide” or an “oligonucleotide molecule” is a single-stranded polynucleotide molecule that may be synthesised to have a user-specified sequence of interest. A “primer” is an example of an oligonucleotide molecule. Typically, at least a portion of an oligonucleotide molecule is specifically or perfectly complementary to a polynucleotide sequence of interest and hybridises to a specifically complementary single-stranded polynucleotide molecule. Oligonucleotide molecules are typically considered to be short polynucleotide molecules; however, their length may be varied. Their length may be suitable for use in at least one of a range of applications including polymerase chain reaction (PCR)-based applications, sequencing applications, molecular cloning and molecular probes.

As used herein the terms “prevent”, “prevention” and “preventing” in the context of a given infection and/or a disease or condition arising from the infection will be understood to mean that a subject has a reduced propensity to develop the infection, and/or disease or condition upon exposure to a pathogenic organism causative of the infection, disease or condition. The reduced propensity to develop the infection and/or disease or condition will be understood to include both a diminished propensity and a lack of any propensity.

As used herein, the term “bacterial strain” is used to refer to a population of bacteria (for example, a bacterial culture), where the vast majority of the bacteria, or effectively all of the bacteria in the population, are descended from a single bacterial organism. Accordingly, the vast majority or effectively all of the bacteria within the population are identical or highly similar to one another, and share a particular phenotype.

As used herein, the term “streptococcal bacterial strain” is used to refer to a bacterial strain belonging to the family Streptococcaceae.

As used herein, the term “subject” includes any animal of economic, social or research importance including bovine, equine, ovine, primate, avian and rodent species. Hence, a “subject” may be a mammal such as, for example, a human or a non-human mammal (e.g., a pig, cat, dog, cow, horse, or sheep). Also included within the scope of this term are laboratory animals (e.g., rodents, rabbits, and the like), birds (e.g., poultry), fish and crustaceans.

The term “synthetic” as applied to polynucleotide molecules is intended to mean that the polynucleotide molecules are produced in vitro, including using bacterial amplification methods.

A “therapeutically effective amount” is at least the minimum concentration or amount required to affect a measurable improvement of a particular disease or condition.

A therapeutically effective amount herein may vary according to factors such as the disease state, age, sex and weight of the patient. A therapeutically effective amount is also one in which any toxic or detrimental effects are outweighed by the therapeutically beneficial effects.

As used herein the terms “treat” and “treating” in the context of a given infection and/or a disease or condition arising from the infection will be understood to encompass reducing the number of pathogenic organisms infecting a subject and/or reducing any symptoms of the infection and/or symptoms of a disease or condition arising from the infection.

As used herein, a “wild-type” form of a given nucleic acid, protein or microorganism (e.g., bacteria) will be understood to encompass naturally occurring forms of the nucleic acid, protein or microorganism and the biological functions that they are capable of.

### DETAILED DESCRIPTION

The following detailed description conveys exemplary embodiments of the present disclosure in sufficient detail to enable those of ordinary skill in the art to practice the present disclosure. Features or limitations of the various embodiments described do not necessarily limit other embodiments of the present disclosure or the present disclosure as a whole. Hence, the following detailed description does not limit the scope of the present disclosure, which is defined only by the claims.

The immunity induced by commercially available streptococcal vaccines is thus inadequate to establish broad immunity against the majority of streptococcal species and/or serotypes within certain pathogenic species.

Vaccination with purified LytC has been shown to result in high Immunoglobulin G (IgG) antibodies and enhanced protection against pneumococcal infection in mice (Wisemann et al., 2001; Corsini et al. 2021). High titres of LytC-specific IgG antibodies have also been observed in serum samples from healthy volunteers in multiple studies, indicating the induction of antibody responses as a result of a natural exposure to S. pneumoniae.

The present disclosure relates to an unexpected finding that the use of an attenuated or killed whole cell Streptococcus vaccine from which the lytC gene has been modified to attenuate, reduce and/or prevent functional LytC expression provides improved vaccine immunogenicity. The present disclosure provides an immunogenic composition comprising an attenuated and/or killed streptococcal bacterial strain comprising a modification that attenuates, reduces or prevents functional LytC expression.

Surprisingly, compared with the parental strain having functional LytC expression, mice immunised with streptococcal bacterial strain harbouring a deletion of lytC produced IgG antibodies with enhanced binding to the surface of encapsulated pneumococcal strains. Further, the antibodies elicited by strains harbouring a deletion of lytC exhibited higher functional opsonophagocytic responses, a recognised surrogate endpoint of pneumococcal vaccine efficacy. These findings unexpectedly illustrate that the immunogenicity of whole cell vaccines may be improved by deleting the lytC gene. This could not be foreseen based on prior studies.

The present disclosure provides compositions capable of inducing cross-protective (i.e., heterotypic) immunity against different streptococcal species and/or different streptococcal serotypes. In an embodiment, the compositions of the disclosure contain killed streptococcal bacteria. In an embodiment, the compositions are an attenuated vaccine. The compositions may also be capable of inducing immunity against a broad range of streptococcal species and/or serotypes, thus reducing the potential impact of serotype replacement.

Also provided herein are methods for manufacturing compositions of the disclosure, as well as medicaments and pharmaceutical compositions comprising the immunogenic or vaccine compositions.

The present disclosure also relates to methods of preventing or treating streptococcal infection in a subject. The methods involve administration of a composition of the disclosure to a subject. The composition may be administered for prophylactic or therapeutic purposes. The methods may induce cross-protective immunity in the subject against a plurality of different streptococcal species and/or serotypes.

### Streptococcal Bacteria

Streptococci are a genus of spheroidal bacteria belonging to the family Streptococcaceae. There are many different species of streptococci, some of which cause disease in humans and/or animals. Others are important in the manufacture of various fermented products.

Individual streptococcal species are classified into two key groups based on their haemolytic properties (alpha- and beta-haemolytic). Alpha-haemolytic streptococci include Streptococcus pneumoniae and Viridans streptococci. The beta-haemolytic group is made up of Group A and Group B streptococci. Group B streptococci usually inhabit the digestive system and the vagina of women without adverse effect. Most people quickly develop a natural immunity to Group B streptococci although they can cause more serious types of infection in newborn infants. Group A streptococci commonly inhabit the throat and skin surface and are a common cause of infection in adults and children. Although most Group A infections do not usually pose a serious threat to health (e.g., throat infections, cellulitis, impetigo, sinusitis, middle ear infections) Group A Streptococci can establish a more serious invasive infection by penetrating deeper into the tissues and organs of the body (e.g., pneumonia, sepsis, meningitis, necrotising fasciitis) and can trigger serious sequelae including acute post-streptococcal glomerulonephritis and acute rheumatic fever.

In addition, Enterococcal (faecal) streptococcal species occur in significant numbers in the bowel and can cause endocarditis and urinary tract infections.

Streptococcus pneumoniae (also called pneumococcus) is an important human pathogen accounting for significant morbidity and mortality. It causes serious conditions including pneumonia, meningitis, bacteraemia, sinusitis, and otitis media. An estimated 1.6 million people die globally from invasive pneumococcal disease each year and approximately one million of those are children. There are many different serotypes of S. pneumoniae (>100) distinguishable on the basis of capsule chemical structure and immunogenicity. The capsular polysaccharide is considered to be an essential virulence factor of S. pneumoniae as non-encapsulated strains are virtually absent among S. pneumoniae that are responsible for invasive pneumococcal disease, and antibodies to capsular polysaccharides are protective against infection by the homologous S. pneumoniae serotype. Capsular polysaccharides are thus used as vaccine antigens in current pneumococcal vaccines.

### Streptococcal Bacterial Strains of the Compositions

The immunogenic compositions of the disclosure are based on attenuated or whole killed streptococcal bacterial strains comprising a modification that attenuates, reduces or prevents functional expression of LytC (or a homologue thereof). The modification may be introduced into any suitable streptococcal bacterial strain.

The streptococcal bacterial strain may, for example, be alpha-, beta-, or gamma-haemolytic streptococci, as classified according to well-characterised haemolytic properties or lack thereof in the case of gamma-haemolytic streptococcal bacteria.

Non-limiting examples of suitable alpha-haemolytic streptococcal bacteria include Streptococcus pneumoniae and viridans streptococci (e.g., S. mutans, S. sanguinis, S. mitis, S. oralis, S. sobrinus, S. millen). Also within the scope of the present disclosure are individual serotypes of these streptococcal species.

Non-limiting examples of suitable beta-haemolytic streptococcal bacteria include those classified under the Lancefield grouping (Groups A-H, L, N and R/S) based on the carbohydrate composition of cell wall bacterial antigens (polysaccharides). For example, the beta-haemolytic bacteria may include any one or more of S. pyogenes (Group A), S. agalactiae (Group B), S. equisimilis (Group C), S. equi (Group C), S. zooepidemicus (Group C), S. dysgalactiae (Group C), Enterococcus faecalis (Group D), S. bovis (Group D), S. milleri (Group E), S. mutans (Group E), S. anginosus (Group F), S. canis (Group G), S. dysgalactiae (Group G), S. sanguis (Group H), S. dysgalactiae (Group L), Lactococcus lactis (Group N), and S. suis (Group R/S). Also within the scope of the present disclosure are individual serotypes of these streptococcal species.

In an embodiment, the attenuated and/or killed streptococcal bacterial strain of the disclosure is of a species selected from the group consisting of Streptococcus agalactiae, Streptococcus bovis, Streptococcus canis, Streptococcus dysgalactiae, Streptococcus equi, Streptococcus equinus, Streptococcus equisimilis, Enterococcus faecalis, Enterococcus faecium, Streptococcus iniae, Streptococcus milleri, Streptococcus mutans, Streptococcus pneumoniae, Streptococcus pyogenes, Streptococcus salivarius, Streptococcus sanguinis, Streptococcus suis, and Streptococcus uberis.

In an embodiment, the attenuated and/or killed streptococcal bacterial strain of the disclosure comprises one or more serotypes of Streptococcus pneumoniae. Accordingly, the compositions may comprise any one of more of S. pneumoniae serotypes 1, 2, 3, 4, 5, 6A, 6B, 6C, 6D, 7A, 7B, 7C, 7F, 8, 9A, 9L, 9N, 9V, 10A, 10B, 10C, 10F, 11A, 11B, 11C, 11D, 11F, 12A, 12B, 12F, 13, 14, 15A, 15B, 15C, 15F, 16A, 16F, 17A, 17F, 18A, 18B, 18C, 18F, 19A, 19B, 19C, 19F, 20, 21, 22A, 22F, 23A, 23B, 23F, 24A, 24B, 24C, 24F, 25A, 25F, 27, 28A, 28F, 29, 31, 32A, 32F, 33A, 33B, 33C, 33D, 33F, 34, 35A, 35B, 35C, 35F, 36A, 36B, 37, 38, 39, 40, 41A, 41F, 42, 43, 44, 45, 46, 47A, 47F, and/or 48.

However, it would be understood by the person skilled in the art that new bacterial strains and serotypes may be discovered. It is to be understood that the attenuated and/or killed streptococcal bacterial strain of the disclosure may comprise streptococcal bacteria or serotypes that are not specifically listed herein. In an embodiment, the composition of the present disclosure comprises any one or more of S. pneumoniae serotypes 1, 2, 3, 4, 5, 6A, 6B, 7F, 8, 9N, 9V, 10A, 11A, 12F, 14, 15B, 17F, 18C, 19A, 19F, 20, 22F, 23F, and 33F.

### Streptococcal Bacterial Derivatives

In an embodiment, the streptococcal bacterial strain is a streptococcal derivative.

The streptococcal derivatives may be recombinant forms of streptococcal bacteria arising from artificial genetic manipulation, or naturally occurring mutant forms of streptococcal bacteria. Without any particular limitation, the streptococcal derivatives may comprise one or more genetic modifications that reduce pathogenicity.

By way of non-limiting example only, the streptococcal derivatives may comprise a genetic alteration which disrupts or removes the capsule locus (cps). For example, any one or more of the S. pneumoniae cpsA, cpsB, cpsC, cpsD and/or cpsE genes, or homologous genes in other streptococcal species, may be modified in order to prevent, disrupt or modify capsule production (e.g., by recombination and the like). Alternatively, the streptococcal derivatives may have spontaneous mutations in these or other genes resulting in naturally occurring non-encapsulated streptococcal bacteria. The streptococcal derivatives may lack all or at least a portion of the capsule locus.

In an embodiment, the streptococcal derivatives lacking a capsule are Streptococcus pneumoniae strain Rx1, or Rx1 derivatives.

Additionally or alternatively, the streptococcal derivatives may comprise a genetic alteration which reduces or prevents the production or activity of other target proteins. By way of non-limiting example only, the genetic alteration may exist in one or more genes encoding a choline-binding protein; one or more genes encoding a further autolysin in addition to lytC (e.g., S. pneumoniae lytA or lytB or homologous genes in other streptococcal bacteria); one or more genes that confer a nutrient/cofactor (e.g., a metal ion) requirement for growth (e.g., S. pneumoniae psaA or homologous genes in other streptococcal bacteria); one or more genes encoding a protective antigen (e.g., S. pneumoniae pspA or homologous genes in other streptococcal bacteria); and/or one or more genes encoding virulence determinants or regulators (e.g., S. pneumoniae codY, comC, comD, cps2A, csp4A, glpO, mgrA, nanA, nanB, pavA, pcpA, phtA, phtB, phtD, phtE, piuA, piaA, ply, prtA, psaA, psrP, rrgA, rrgB, spxB, and homologs of these genes in other streptococcal bacteria).

Additionally or alternatively, the streptococcal derivatives may comprise a genetic alteration resulting in an auxotroph with reduced pathogenicity and/or growth in vivo. By way of non-limiting example only, the genetic alteration may exist in one or more genes encoding a thymidylate synthase.

Additionally or alternatively, the streptococcal derivatives may comprise one or more (external) genes from a streptococcal bacterium that is of the same species but a different serotype; a streptococcal bacterium that is from a different species; a non-streptococcal bacterium; or a human or a non-human mammal (e.g., a pig, cat, dog, cow, horse, or sheep); a laboratory animal (e.g., a rodent or rabbit); a bird; and/or a subject to which the recombinant streptococcal bacteria are to be administered. In an embodiment, the external gene or genes disrupt or otherwise inactivate one or more endogenous gene or genes (e.g., any one or more genes as set out in the paragraph directly above). In other embodiments, the external gene or genes do not disrupt or inactivate any endogenous gene. By way of non-limiting example only, the external gene or genes encode proteins that induce or enhance an immune response in a subject to which the streptococcal derivatives are administered. The immune response may be innate, adaptive, or both. In an embodiment, the external gene or genes encode an immunomodulator (e.g., a cytokine, chemokine, antibody, fusion protein, peptide, protein, and/or hormone). In other embodiments, the external gene or genes may comprise an antigen from another different family of bacteria (e.g., a Mycoplasma pneumoniae antigen, a Haemophilus influenzae antigen, a Chlamydophila pneumoniae antigen, a Moraxella catarrhalis antigen, a Staphylococcus aureus antigen), a viral antigen (e.g., an adenovirus antigen, a coronavirus antigen, an influenza virus antigen, a parainfluenza virus antigen, a metapneumovirus antigen, a rhinovirus antigen, a respiratory syncitial virus antigen, an HIV antigen, a hepatitis virus antigen, or a herpes virus antigen, a measles virus antigen, a mumps virus antigen, a papillomavirus virus antigen, a rubella virus antigen, a Varicella Zoster virus antigen), a fungal/yeast antigen, a helminthic antigen, and/or a protozoan antigen.

Additionally or alternatively, the streptococcal derivatives may comprise a genetic alteration causing the bacteria to overexpress one or more target genes. In this context, “overexpression” will be understood to mean a level of expression that is increased compared to expression of the same gene without the genetic modification in a corresponding streptococcal bacterium, under the same biological conditions. The overexpression of a given target gene may, for example, induce or enhance an immune response in a subject against streptococcal strains that are parental to the streptococcal derivatives administered and/or against the streptococcal derivatives themselves. By way of non-limiting example only, the genetic alteration may increase the production of one or more genes in the streptococcal derivatives encoding a protein capable of activating the complement system (e.g., S. pneumoniae cbpA, pspA, ply, or homologs of these genes in other streptococcal bacteria).

Additionally or alternatively, the streptococcal derivatives may comprise a genetic alteration causing defective DNA repair capacity. In an embodiment, the use of compositions of the disclosure comprising photon-irradiated (e.g., gamma-irradiated and/or X-irradiated) streptococcal derivatives with decreased capacity to repair DNA lesions arising from photon-irradiation may be advantageous insofar as the dose of photon-irradiation needed for attenuation or inactivation can be reduced, while vaccine efficacy and safety can conversely be increased. In an embodiment, the streptococcal derivatives comprise a genetic alteration that disrupts or inactivates expression of one or more genes encoding a protein in a mismatch repair system (e.g., S. pneumoniae hex locus or homologues of this locus in other streptococcal bacteria). In other embodiments, the streptococcal derivatives comprise a genetic alteration that disrupts or inactivates expression of one or more genes encoding a DNA alkylation repair protein (e.g., S. pneumoniae DNA polymerase 4, hexA, hexB, mutS, radC, recA, recF, recN, recO, uvrA, uvrB, uvrC, uvrD or homologues of these genes in other streptococcal bacteria).

Additionally or alternatively, the streptococcal derivatives may comprise a genetic alteration that facilitates production of a double-stranded RNA (dsRNA). The dsRNA may be mRNA or tRNA. Without limitation, the length of the dsRNA may be more than 10, more than 15, more than 20, more than 25, more than 30, more than 35, more than 40, more than 45, more than 50, more than 55, more than 60, more than 65 or more than 70 base pairs in length. Additionally or alternatively, the length of the dsRNA may be: between about 10 and about 70 base pairs (bp); between about 10 and about 50 base pairs (bp); between about 10 and about 30 base pairs (bp); between about 20 and about 70 base pairs (bp); between about 20 and about 60 base pairs (bp); between about 20 and about 50 base pairs (bp); between about 20 and about 40 base pairs (bp); between about 20 and about 30 base pairs (bp); between about 30 and about 70 base pairs (bp); between about 40 and about 70 base pairs (bp); between about 50 and about 70 base pairs (bp); between about 60 and about 70 base pairs (bp); between about 30 and about 60 base pairs (bp); between about 30 and about 50 base pairs (bp); or between about 30 and about 40 base pairs (bp); in length. In an embodiment, the dsRNA is a component of a larger RNA molecule that is otherwise single-stranded. The larger RNA molecule may comprise multiple dsRNA components. The dsRNA may be an internal component or an end component of the larger RNA molecule. In an embodiment, the dsRNA may comprise a termination stem-loop sequence. The dsRNA may arise from a region of self-complementarity within the larger RNA molecule. Coding region(s)/exon(s) within a given gene of a streptococcal derivative can be engineered to include one or more region(s) of self-complementarity and thereby produce a dsRNA portion when transcribed.

The dsRNA may be capable of recognition by Toll-like receptor (TLR) proteins expressed by cells in a subject to which the streptococcal derivatives are administered. The TLR proteins may be located in the endoplasmic reticulum and/or endosomal compartment of the cells. The TLR proteins may be Toll-like receptor 3 (TLR3) proteins. Without limitation, the cells may be any one or more of B lymphocytes, T lymphocytes, natural killer cells and/or dendritic cells. Recognition of the dsRNA by the TLR3 protein may induce an immune response in the subject. The immune response may be an innate immune response. The immune response may be an interferon type-1 response and/or comprise the release of inflammatory cytokines.

In general, streptococcal derivatives used in compositions of the disclosure will have a significant degree of genetic similarity to the parent strain from which they derive. By way of non-limiting example, a “streptococcal derivative” as referred to herein may have more than 70%, more than 75%, more than 80%, more than 85%, more than 90%, more than 92%, more than 94%, more than 95%, more than 96%, more than 97%, more than 98%, or more than 99% sequence homology to the parent streptococcal strain from which it is derived. By way of further non-limiting example, a “streptococcal derivative” as referred to herein may comprise a genetic alteration in one, two, three, four, five or more than five genes or regulatory sequences necessary for expression of those genes, when compared to corresponding of its parent strain. The genetic alteration may increase, decrease, or prevent expression of the gene or genes in question.

Techniques for the genetic manipulation of bacteria are well known to those of ordinary skill in the art (see, for example, Vennison “Laboratory Manual for Genetic Engineering”, PHI Learning Pvt. Ltd., 2010; Zyskind and Bernstein, “Recombinant DNA Laboratory Manual”, Elsevier, 2014; Bose, “Genetic Manipulation of Staphylococci” in “Methods in Molecular Biology”, Springer Protocols, volume 1106, pages 101-111, 2014; Hakenbeck and Chhatwal, “Molecular Biology of Streptococci”, Horizon Scientific Press, 2007; Morona et al., “The effect that mutations in the conserved capsular polysaccharide biosynthesis genes cpsA, cpsB and cpsD have on virulence of Streptococcus pneumoniae”, J. Infect. Dis. 189: 1905-1913, 2004; Morona et al., “Mutational analysis of the carboxy-terminal [YGX]₄ repeat domain of CpsD, an autophosphorylating tyrosine kinase required for capsule biosynthesis in Streptococcus pneumonia”, J. Bacteriol. 185: 3009-3019, 2003; McAllister et al., “Molecular analysis of the psa permease complex of Streptococcus pneumoniae”, Mol. Microbiol. 53:889-901, 2004; Mahdi et al., “Identification of a novel pneumococcal vaccine antigen preferentially expressed during meningitis in mice”, J. Clin. Invest. 122:2208-2220, 2012.

In one non-limiting embodiment, wherein the attenuated and/or killed streptococcal bacterial strain comprises a S. pneumoniae strain derived from Rx1. Rx1 is a capsule-negative mutant derived from a S. pneumoniae serotype 2 strain D39.

In an embodiment, the attenuated and/or killed streptococcal bacterial strain further comprises modification or deletion of genes selected from the group consisting of lytA, ply, and psaA.

In an embodiment, the attenuated and/or killed streptococcal bacterial strain further comprises a modification or deletion of a lytA gene. The autolysin gene (lytA) gene may be deleted or rendered non-functional in the Rx1 derivative strain.

Additionally or alternatively, the pneumolysin gene (ply) may be deleted or rendered non-functional in the Rx1 derivative strain. For example, the ply gene may be replaced with another gene such as a toxoid version of ply. In an embodiment, the attenuated and/or killed streptococcal bacterial strain further comprises a modification or deletion of a ply gene. In an embodiment, the attenuated and/or killed streptococcal bacterial strain further comprises a toxoid version of ply designated PdT.

In an embodiment, the modification that attenuates, reduces or prevents functional LytC expression may be introduced into Rx1 (ΔLytA, PdT). GPN-001 as described herein is an example of a Rx1 (ΔLytA, PdT) strain. A method of deriving a strain having ΔLytA, PdT modifications is described in PCT publication No. WO2016/149771, which is herein incorporated in its entirety.

Additionally or alternatively, the pneumococcal surface antigen A gene (psaA) gene may be deleted or otherwise rendered non-functional. The psaA gene of S. pneumoniae encodes the pneumococcal surface antigen A (PsaA) involved in Mn²⁺ transport and resistance to oxidative stress. A psaA-deletion mutant as described herein may be defective in growth in low Mn²⁺ environments and/or defective in pneumococcal competence.

In an embodiment, the modification that attenuates, reduces or prevents functional LytC expression may be introduced into a Rx1 (ΔLytA, PdT, ΔPsaA). GPN-002 as described herein is an example of a Rx1 (ΔLytA, PdT, ΔPsaA) strain. A method of deriving a strain having ΔPsaA modification is described in United States publication No. 2021-0361757, which is herein incorporated in its entirety. Such strains may have reduced virulence, reduced competence, and/or reduced growth in low Mn²⁺ environments. Furthermore, fermentor growth of strains having ΔLytA, PdT, ΔpsaA modifications under Mn²⁺ stress conditions may induce changes in gene expression that increases the level of production of protective antigens. An improved level of protection may thus be afforded by use of immunogenic composition with higher level of expression of protective antigens.

In an embodiment, the attenuated and/or killed streptococcal bacterial strain further comprises a modification or deletion of a lytA gene, a modification or deletion of a ply gene and a modification or deletion of a psaA gene, and further comprises a toxoid version of ply designated PdT.

### LytC

The present disclosure relates to an immunogenic composition comprising an attenuated and/or killed streptococcal bacterial strain comprising a modification that attenuates, reduces or prevents functional expression of LytC (or a homologue thereof). In an embodiment, the modification attenuates, reduces or prevents functional expression of LytC.

LytC is a cell wall hydrolase that has been proposed to contribute to fratricide with LytA by facilitating the lysis of non-competent pneumococcal cells (Eldhom et al., 2009). LytC also plays a role in biofilm formation and adherence to the nasopharyngeal epithelium. The activity of the LytC enzyme is highest at 30° C., suggesting that the physiological role of this enzyme may be to facilitate colonisation of the pneumococcus in the upper respiratory tract (Tomasz et al., 1975). Given its surface localisation and the importance of this enzyme in key virulence traits, LytC has been investigated as a streptococcal vaccine candidate in animal studies. Vaccination with purified LytC validated the potential of this protein as a protective immunogen, with immunised mice exhibiting high Immunoglobulin G (IgG) antibodies and enhanced protection against pneumococcal infection in mice (Wisemann et al., 2001; Corsini et al. 2021). High titres of LytC-specific IgG antibodies have also been observed in serum samples from healthy volunteers in multiple studies, indicating the induction of antibody responses as a result of a natural exposure to S. pneumoniae (Holmlund et al. 2009; Croucher et al., 2017).

The modification of LytC may be any suitable modification providing it attenuates, reduces or prevents functional expression of LytC (or a homologue thereof). For example, the lytC gene may be modified using any suitable method known to those skilled in the art.

For example, the gene may be modified as described herein; however, it is to be understood that other suitable methods of modifying the lytC gene may be utilised to produce a composition of the present disclosure. Techniques for the genetic manipulation of bacteria are well known to those of ordinary skill in the art as described elsewhere herein.

In an embodiment, the modification may prevent functional expression of LytC (or a homologue thereof). For example, the gene may be modified to prevent expression of LytC (or a homologue thereof), for example, by introducing a nonsense mutation such that translation of LytC is terminated so that any resulting protein expressed from the modified gene is truncated and not functional. Alternatively, the gene may be deleted, for example, using methods described herein. In an embodiment, the gene may be modified such that the translated protein is a variant that has reduced or attenuated function. Alternatively, the expression level of LytC may be reduced or attenuated, for example, by modifying the control sequences that facilitate expression levels of LytC (or a homologue thereof) as would be understood by a person skilled in the art.

The modification that attenuates, reduces or prevents functional expression of LytC (or a homologue thereof) may be made to any streptococcal bacterial strain having a lytC gene or a homologue thereof. In an embodiment, the modification to lytC or homologue thereof may be made to any suitable serotype of a species selected from the group consisting of Streptococcus agalactiae, Streptococcus bovis, Streptococcus canis, Streptococcus dysgalactiae, Streptococcus equi, Streptococcus equinus, Streptococcus equisimilis, Enterococcus faecalis, Enterococcus faecium, Streptococcus iniae, Streptococcus milleri, Streptococcus mutans, Streptococcus pneumoniae, Streptococcus pyogenes, Streptococcus salivarius, Streptococcus sanguinis, Streptococcus suis and Streptococcus uberis.

In an embodiment, attenuated and/or killed streptococcal bacterial strain comprising a modification that attenuates, reduces or prevents functional LytC expression is a Rx1 (ΔlytA, PdT, ΔpsaA, ΔlytC) strain. GPN-003 as described herein is an example of a Rx1 (ΔlytA, PdT, ΔpsaA, ΔlytC) strain. Methods of deriving a strain having a ΔlytC are described herein.

Target Streptococcal Species and/or Serotype Thereof

In an embodiment, the compositions of the present invention induce an immune response to at least one target streptococcal species and/or serotype thereof. The target streptococcal species and/or serotype thereof may be pathogenic bacteria capable of establishing a detrimental infection in a host organism.

In an embodiment, the at least one target streptococcal bacterial species and/or serotype thereof is selected from the group consisting of: Streptococcus agalactiae, Streptococcus bovis, Streptococcus canis, Streptococcus dysgalactiae, Streptococcus equi, Streptococcus equinus, Streptococcus equisimilis, Enterococcus faecalis, Enterococcus faecium, Streptococcus iniae, Streptococcus milleri, Streptococcus mutans, Streptococcus pneumoniae, Streptococcus pyogenes, Streptococcus salivarius, Streptococcus sanguinis, Streptococcus suis and Streptococcus uberis.

In an embodiment, the at least one target streptococcal bacterial species and/or serotype thereof of the disclosure comprises one or more serotypes of Streptococcus pneumoniae. Accordingly, the compositions may comprise any one of more of S. pneumoniae serotypes 1, 2, 3, 4, 5, 6A, 6B, 6C, 6D, 7A, 7B, 7C, 7F, 8, 9A, 9L, 9N, 9V, 10A, 10B, 10C, 10F, 11A, 11B, 11C, 11D, 11F, 12A, 12B, 12F, 13, 14, 15A, 15B, 15C, 15F, 16A, 16F, 17A, 17F, 18A, 18B, 18C, 18F, 19A, 19B, 19C, 19F, 20, 21, 22A, 22F, 23A, 23B, 23F, 24A, 24B, 24C, 24F, 25A, 25F, 27, 28A, 28F, 29, 31, 32A, 32F, 33A, 33B, 33C, 33D, 33F, 34, 35A, 35B, 35C, 35F, 36A, 36B, 37, 38, 39, 40, 41A, 41F, 42, 43, 44, 45, 46, 47A, 47F, and/or 48.

However, it would be understood by the person skilled in the art that new bacterial strains and serotypes may be discovered. It is to be understood that the attenuated and/or killed streptococcal bacterial strain of the disclosure may comprise streptococcal bacteria or serotypes that are not specifically listed herein.

In an embodiment, the compositions of the present invention induce an immune response to a plurality of target streptococcal species and/or serotype thereof. In an embodiment, the compositions of the present invention induce a cross-protective immune response to at least one target streptococcal species and/or serotype thereof. In an embodiment, the compositions of the present invention induce a cross-protective immune response to a plurality of target streptococcal species and/or serotypes thereof.

### Culturing of Streptococcal Bacterial Strains

The attenuated and/or killed streptococcal bacterial strain of the present disclosure may be cultured using any suitable standard techniques known to those skilled in the art. For example, the attenuated and/or killed streptococcal bacterial strain may be cultured under standard conditions in a range of suitable media, for example, Soytone broth, Todd-Hewitt Yeast (THY) broth, etc, for a suitable period of time under suitable culturing conditions until a desired cell density or optical density is obtained.

### Killing of Streptococcal Bacterial Strains

The streptococcal bacterial strains of the present disclosure may be killed using any suitable methods known in the art, providing that the killed bacteria are suitable for use as a composition of the present disclosure. For example, the streptococcal bacterial strains may be killed by suitable protocols for chemical treatment, thermal treatment, irradiation treatment, high hydrostatic pressure, pulsed electric field, ultrashort pulsed laser, ultrasound under pressure, UV-irradiation, or microbial inactivation. In an embodiment, the streptococcal bacterial strains of the present disclosure may be killed by photon-irradiation.

### Photon Radiation

Streptococcal bacteria in compositions of the disclosure may be exposed to photon-radiation. As noted above, the term “photon-radiation” will be understood to encompass both gamma-radiation (i.e., gamma-rays) and X-radiation (i.e., X-rays). Accordingly, “photon-irradiated” streptococcal bacterial strain of the present disclosure may be “gamma-irradiated” by way of exposure to gamma-radiation (i.e., gamma-rays), “X-irradiated” by way of exposure to X-radiation (i.e., X-rays), or both. As known to those of ordinary skill in the art, X-rays are identical to gamma-rays except they are emitted by the passage of electrons through an electric field of a nucleus rather than the nucleus itself upon radioactive decay. By way of non-limiting example only, to become photon-irradiated a material may be subjected to photon-radiation at energies of at least 0.01 MeV, at least 0.1 MeV, at least 0.5 MeV, between 0.01 MeV and 0.5 MeV, between 0.01 MeV and 1 MeV, between 0.01 MeV and 10 MeV, between 0.5 MeV and 20 MeV, between 0.5 MeV and 15 MeV, between 0.5 MeV and 10 MeV, between 0.5 MeV and 5 MeV, between 0.5 MeV and 2 MeV, or between 1 MeV and 2 MeV (e.g., 1.25 MeV).

Streptococcal bacteria in compositions of the disclosure may be gamma-irradiated. Any suitable source of gamma-radiation may be used. Suitable gamma emitters include, but are not limited to Ba¹³⁷, Co⁶⁰, Cs¹³⁷, Ir¹⁹², U²³⁵, Se⁷⁵ and Yb¹⁶⁹.

Gamma-irradiation of the streptococcal bacteria of the disclosure may be performed using commercially available devices, for example, a Gammacell irradiator manufactured by Atomic Energy of Canada Ltd., Canada (e.g., Gammacell 40 Irradiator, Gammacell 220 Irradiator, Gammacell 1000 irradiator, Gammacell 3000 irradiator), a gamma-irradiator manufactured by J. L. Shepherd and Associates (San Fernando, California, USA), or a Nordion Gamma Cell-1000 irradiator manufactured by Nordion Inc. (Kanata, Ontario, Canada). Other suitable devices are described, for example, in U.S. Pat. Nos. 3,557,370 and 3,567,938.

Additionally or alternatively, streptococcal bacteria of the disclosure may be X-irradiated. Any suitable source of X-radiation may be used. Suitable sources of X-radiation include, but are not limited to, the eXelis® sterilization X-ray machine manufactured by IBA Industrial (Louvain-la-Neuve, Belgium). Other suitable devices include for example, the RS2400® and RS3400® manufactured by Rad Source Technologies Inc. (Suwanee, Georgia, USA).

In general, the streptococcal bacteria of the disclosure are exposed to a dose of photon-radiation (e.g., gamma-radiation and/or X-radiation) sufficient to attenuate or inactivate the streptococcal bacterial strain of the present disclosure. Preferably, the dose of photon-radiation is sufficient to attenuate or inactivate the bacteria without substantially disrupting the structure of antigens (e.g., surface antigens). The immunogenicity of antigenic determinants may therefore be retained by the photon-irradiated bacteria and their derivatives. Preferably, the dose of photon-radiation is administered to the bacteria over a period of time and at a level sufficient to ensure that all streptococcal bacterial strain of the present disclosure under treatment are exposed without adversely affecting the structural integrity of antigenic determinants.

As known to those of ordinary skill in the art, a measure for an absorbed dose of radiation is the gray (Gy), which is defined as 1 joule of energy deposited in 1 kilogram of mass. An old unit of measure for this is the rad, which stands for “radiation absorbed dose”, where 1 Gy=100 rad.

Streptococcal bacteria for use in accordance with the present disclosure may be exposed to a total dose of photon-radiation (e.g., gamma-radiation and/or X-radiation) in the range of about 1×10³ rad and about 2×10⁹ rad (or about 10 Gy to about 2×10⁴ kGy). In certain embodiments of the disclosure, the streptococcal bacteria or derivatives are exposed to a total dose of X-radiation and/or gamma-radiation of between about 1×10³ rad and about 2×10⁹ rad, between about 1×10³ rad and about 1×10⁹ rad, between about 1×10³ rad and about 1×10⁸ rad, between about 1×10³ rad and about 1×10⁷ rad, between about 1×10³ rad and about 1×10⁶ rad, between about 1×10³ rad and about 1×10⁵ rad, between about 1×10³ rad and about 1×10⁴ rad, between about 1×10³ rad and about 2×10⁹ rad, between about 1×10⁴ rad and about 2×10⁹ rad, between about 1×10⁵ rad and about 2×10⁹ rad, between about 1×10⁶ rad and about 2×10⁹ rad, between about 1×10⁷ rad and about 2×10⁹ rad, between about 1×10⁸ rad and about 2×10⁹ rad or between about 1×10⁹ rad and about 2×10⁹ rad.

In an embodiment of the disclosure, the streptococcal bacteria of the present disclosure are exposed to a total dose of photon-radiation (e.g., X-radiation and/or gamma-radiation) of between about 6.5×10⁴ rad and about 2×10⁷ rad (about 0.65 kGy to about 200 kGy). In other embodiments of the disclosure, the streptococcal bacterial strain of the present disclosure are exposed to a total photon-radiation dose of about 10 kGy to about 12 kGy, about 12 kGy to about 14 kGy, about 14 kGy to about 16 kGy, about 10 kGy to about 20 kGy, about 14 kGy to about 20 kGy, about 20 kGy to about 30 kGy, about 20 kGy to about 25 kGy, about 25 kGy to about 30 kGy, about 30 to 35 kGy, about 10 kGy, about 11 kGy, about 12 kGy, about 13 kGy, about 14 kGy, about 15 kGy, about 16 kGy, about 17 kGy, about 18 kGy, about 19 kGy, about 20 kGy, about 21 kGy, about 22 kGy, about 23 kGy, about 24 kGy, about 25 kGy, about 26 kGy, about 27 kGy, about 28 kGy, about 29 kGy, about 30 kGy, about 31 kGy, about 32 kGy, about 33 kGy, about 34 kGy, about 35 kGy, about 20 kGy, about 20 kGy, more than 10 about 12 kGy to about 14 kGy, more than 12 kGy, more than 14 kGy, more than 16 kGy, more than 18 kGy, more than 20 kGy, more than 22 kGy, more than 24 kGy, more than 26 kGy, more than 28 kGy, more than 30 kGy, more than 35 kGy, more than 40 kGy, 1.26×10⁶ rad (12.6 kGy), a total photon-radiation dose of about 1×10⁶ rad (about 10 kGy) photon-rays, or a total photon-radiation dose of about 1×10⁵ rad (1 KGy).

The optimal dose of photon-radiation (e.g., gamma-radiation and/or X-radiation) may be influenced by factors such as the medium in which the streptococcal bacteria of the present disclosure are present, the number of bacteria present to be treated, the temperature of the bacteria present to be treated (e.g., frozen on dry ice or at room temperature), water availability, oxygen availability and/or the subtype or strain under treatment. Accordingly, the total dose of photon-radiation, the exposure time and/or the level of photon-radiation applied over the period of exposure may be optimised to enhance the effectiveness of the treatment.

The total dose of photon-radiation (e.g., X-radiation and/or gamma-radiation) may be administered to the streptococcal bacterial strain of the present disclosure cumulatively over a period of time. For example, photon-radiation on may be administered to the streptococcal bacterial strain of the present disclosure at a level lower than that of the total dose, over a time period sufficient to achieve the total dose of photon-radiation required.

In one embodiment, a streptococcal bacterial strain of the disclosure may be cultured in a suitable bacterial culture media that supports the growth of streptococcal strains, such as THY broth, soytone broth, etc, to obtain a suitable cell density, for example, a cell density of between 10⁴ to 10⁸ colony forming units (CFU)/ml. In an embodiment, the cell density is between 10⁷ to 10⁸ colony forming units (CFU)/ml. In an embodiment, the cell density is approximately 10⁸ colony forming units (CFU)/ml. The bacteria may be concentrated, for example, using techniques known to those skilled in the art, such as centrifugation, filtration, etc. In an embodiment, the bacteria are washed with a suitable solution, such as PBS, however, a person skilled in the art would appreciate that various buffered solutions would be suitable. Techniques such as tangential flow filtration, diafiltration, etc may be utilised to wash the bacteria. In an embodiment, the bacterial cells are re-centrifuged and resuspended in PBS with 5% to 20% glycerol. In an embodiment, the glycerol concentration is selected from 0%, 1%, 2%, 3%, 4%, 5%, 6%, 7%, 8%, 9%, 10%, 11%, 12%, 13%, 14%, 15%, 16%, 17%, 18%, 19% or 20%. In an embodiment, the bacterial cells are resuspended at a preparation concentration of between 1×10⁴ and 1×10¹² CFU/ml, for example, 1×10⁴, 1×10⁵, 1×10⁶, 1×10⁷, 1×10⁸, 1×10⁹, 1×10¹¹, or 1×10¹² CFU/ml. In an embodiment, the preparation concentration is 1×10¹⁰ CFU/ml. The streptococcal bacterial strain preparation may be photon-irradiated at various doses of irradiation (0.5-30 kGy) and temperature conditions, on ice or dry ice (DI) as described herein. Alternatively, the washed bacterial cells maybe lyophilised prior to photon-irradiation using standard techniques known to those skilled in the art.

In one embodiment, preparations of streptococcal bacterial strain of the present disclosure are maintained in a frozen and/or lyophilised state while being exposed to photon-radiation (e.g., gamma-radiation and/or X-radiation). This may facilitate the preservation of biological integrity and avoid unnecessary damage of antigens thereby enhancing the immunogenicity of photon-irradiated bacterial preparations, and in particular, their ability to elicit cross-reactive/cross-protective immunity against, for example, heterologous subtypes. In general, a photon-radiation dose of 10-30 kGy (as described herein) may be effective for treating preparations of frozen and/or lyophilised streptococcal bacterial strains of the present disclosure. Alternatively, a photon-radiation dose of 20-40 kGy (e.g., more than 20, more than 22, more than 24, more than 26, more than 28, more than 30, more than 32, more than 34, more than 36, or more than 38 kGy, etc) may be effective for treating preparations of frozen and/or lyophilised streptococcal bacterial strain of the present disclosure.

As mentioned above, it is preferable that treatment with photon-radiation is sufficient to inactivate the streptococcal bacterial strain of the present disclosure without substantially disrupting the structure of bacterial antigens. Attenuation and/or inactivation of the streptococcal bacterial strain of the present disclosure may be assessed using methods generally known in the art.

For example, bacterial attenuation and/or inactivation can be assessed by determining the number of viable bacteria that form colonies on agar media (i.e., the colony forming units) after being treated with photon-radiation (e.g., gamma-radiation and/or X-radiation).

The integrity of antigenic determinants can be assessed, for example, by reactivity with panels of monospecific antisera raised against purified native antigenic components using Western blotting, FACs analysis, or enzymatic assays of surface components.

Attenuated Streptococcal Strains Having Modified lytC

In an embodiment, the bacterial strain comprising a modification that attenuates, reduces or prevents functional expression of LytC (or a homologue thereof) is attenuated. That is, the strain is non-pathogenic, and accordingly does not cause disease when inoculated as a live strain into mice. In an embodiment, the composition of the present disclosure comprises an attenuated whole cell immunogenic composition.

In an embodiment, an attenuated whole cell immunogenic composition of the present disclosure may be used as a live attenuated immunogenic or vaccine composition. In an embodiment, the attenuated immunogenic or vaccine composition induces an immune response to at least one target streptococcal species and/or serotype thereof. In an embodiment, the attenuated immunogenic or vaccine composition improves effectiveness of an immune response compared to the parental strain lacking the modification. In an embodiment, the attenuated streptococcal bacterial strain is derived from a parental strain lacking the modification, and the attenuated streptococcal bacterial strain has improved stability compared to the parent strain.

### Immune Response

The composition of the disclosure may induce an immune response when administered to a subject. In an embodiment, the composition may induce a heterotypic immune response. In an embodiment, the composition induces a cross-protective immune response.

In an embodiment, the attenuated and/or killed streptococcal bacterial strain comprising the lytC modification may induce an immune response with improved effectiveness compared to the parental strain. For example, the composition may induce increased protection from infection and/or disease compared to a composition comprising the parental strain. In an embodiment, the composition of the disclosure induces a higher total level of antibodies. In an embodiment, the composition of the disclosure induces a higher total level of IgG antibodies. In an embodiment, the composition induces an increase or decrease in a particular subtype of immunoglobulin (e.g., selected from IgG1, IgG2a, IgG2b, IgG3, IgG4, etc, IgA, IgE, IgM etc). In an embodiment, the composition induces a change in the ratio of one immunoglobulin subtype compared to another immunoglobulin subtype, for example, a change in a ratio selected from IgG1:IgG2a, IgG1:IgG2b, IgG1:IgG3, etc. The change may be an increase or a decrease. In an embodiment, the composition enhances the antibody response to opsonise and kill target streptococcal species and/or serotypes thereof. In an embodiment, the composition enhances the antibody response to opsonise and kill encapsulated pneumococci. In an embodiment, the composition of the disclosure induces an enhanced cellular response, such as a shift in the Th1/Th2 bias.

### Prophylactic and Therapeutic Methods

The disclosure provides prophylactic methods for the prevention of streptococcal infection in a subject. Also provided are therapeutic methods for treating streptococcal infection in a subject. The methods comprise administering an immunogenic or vaccine composition comprising an attenuated and/or killed streptococcal bacterial strain comprising a modification that attenuates, reduces or prevents functional expression of LytC (or a homologue thereof) to the subject, for example, in the form of a vaccine of the disclosure.

The methods induce or enhance the immune response against target streptococcal bacteria in the subject. The immune response may be cross-protective/heterologous insofar as it may induce or enhance the immune response against multiple serotypes of streptococcal bacteria. The methods may also comprise administering multiple different attenuated and/or killed streptococcal bacterial strain comprising the lytC modifications to thereby generate immunity against multiple species of streptococcal bacteria and various serotypes thereof.

The methods may induce or enhance an immune response against any one or more of the following target streptococcal species and/or serotype thereof: Streptococcus agalactiae, Streptococcus bovis, Streptococcus canis, Streptococcus dysgalactiae, Streptococcus equi, Streptococcus equinus, Streptococcus equisimilis, Enterococcus faecalis, Enterococcus faecium, Streptococcus iniae, Streptococcus milleri, Streptococcus mutans, Streptococcus pneumoniae, Streptococcus pyogenes, Streptococcus salivarius, Streptococcus sanguinis, Streptococcus suis and Streptococcus uberis.

In an embodiment, the methods comprise preventing or treating a streptococcal infection caused by a target streptococcal species and/or serotype thereof by administering to the subject a composition comprising an attenuated and/or killed streptococcal bacterial strain comprising a lytC modification of the same streptococcal species.

In an embodiment, the streptococcal species or serotype causative of the infection may be different to the attenuated and/or killed streptococcal bacterial strain comprising a modification that attenuates, reduces or prevents functional expression of LytC (or a homologue thereof).

By way of non-limiting example only, the methods may be used to prevent or treat:
(i) an infection, disease or condition caused by any one or more serotypes of Streptococcus agalactiae by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Streptococcus agalactiae. The attenuated and/or killed Streptococcus agalactiae serotype(s) administered may differ from the serotype(s) causative of the infection, disease or condition;
(ii) an infection, disease or condition caused by any one or more serotypes of Streptococcus bovis by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Streptococcus bovis. The attenuated and/or killed Streptococcus bovis serotype(s) administered may differ from the serotype(s) causative of the infection, disease or condition;
(iii) an infection, disease or condition caused by any one or more serotypes of Streptococcus canis by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Streptococcus canis. The attenuated and/or killed Streptococcus canis serotype(s) administered may differ from the serotype(s) causative of the infection, disease or condition;
(iv) an infection, disease or condition caused by any one or more serotypes of Streptococcus dysgalactiae by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Streptococcus dysgalactiae. The attenuated and/or killed Streptococcus dysgalactiae serotype(s) administered may differ from the serotype(s) causative of the infection, disease or condition;
(v) an infection, disease or condition caused by any one or more serotypes of Streptococcus equi by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Streptococcus equi. The attenuated and/or killed Streptococcus equi serotype(s) administered may differ from the serotype(s) causative of the infection, disease or condition;
(vi) an infection, disease or condition caused by any one or more serotypes of Streptococcus equinus by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Streptococcus equinus. The attenuated and/or killed Streptococcus equinus serotype(s) administered may differ from the serotype(s) causative of the infection, disease or condition;
(vii) an infection, disease or condition caused by any one or more serotypes of Streptococcus agalactiae by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Streptococcus agalactiae. The attenuated and/or killed Streptococcus agalactiae serotype(s) administered may differ from the serotype(s) causative of the infection, disease or condition;
(viii) an infection, disease or condition caused by any one or more serotypes of Streptococcus equisimilis by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Streptococcus equisimilis. The attenuated and/or killed Streptococcus equisimilis serotype(s) administered may differ from the serotype(s) causative of the infection, disease or condition;
(ix) an infection, disease or condition caused by any one or more serotypes of Enterococcus faecalis by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Enterococcus faecalis. The attenuated and/or killed Enterococcus faecalis serotype(s) administered may differ from the serotype(s) causative of the infection, disease or condition;
(x) an infection, disease or condition caused by any one or more serotypes of Enterococcus faecium by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Enterococcus faecium. The attenuated and/or killed Enterococcus faecium serotype(s) administered may differ from the serotype(s) causative of the infection, disease or condition;
(xi) an infection, disease or condition caused by any one or more serotypes of Streptococcus iniae by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Streptococcus iniae. The attenuated and/or killed Streptococcus iniae serotype(s) administered may differ from the serotype(s) causative of the infection, disease or condition;
(xii) an infection, disease or condition caused by any one or more serotypes of Streptococcus milleri by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Streptococcus milleri. The attenuated and/or killed Streptococcus milleri serotype(s) administered may differ from the serotype(s) causative of the infection, disease or condition;
(xiii) an infection, disease or condition caused by any one or more serotypes of Streptococcus mutans by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Streptococcus mutans. The attenuated and/or killed Streptococcus mutans serotype(s) administered may differ from the serotype(s) causative of the infection, disease or condition;
(xiv) an infection, disease or condition caused by any one or more serotypes of Streptococcus pneumoniae by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Streptococcus pneumoniae. The attenuated and/or killed Streptococcus pneumoniae serotype(s) administered may differ from the serotype(s) causative of the infection, disease or condition;
(xv) an infection, disease or condition caused by any one or more serotypes of Streptococcus pyogenes by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Streptococcus pyogenes. The attenuated and/or killed Streptococcus pyogenes serotype(s) administered may differ from the serotype(s) causative of the infection, disease or condition;
(xvi) an infection, disease or condition caused by any one or more serotypes of Streptococcus salivarius by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Streptococcus salivarius. The attenuated and/or killed Streptococcus salivarius serotype(s) administered may differ from the serotype(s) causative of the infection, disease or condition;
(xvii) an infection, disease or condition caused by any one or more serotypes of Streptococcus sanguinis by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains comprise one or more serotype(s) of Streptococcus sanguinis. The attenuated and/or killed Streptococcus sanguinis serotype(s) administered may differ from the serotype(s) causative of the infection, disease or condition;
(xviii) an infection, disease or condition caused by any one or more serotypes of Streptococcus suis by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains are one or more serotype(s) of Streptococcus suis. The attenuated and/or killed Streptococcus suis serotype(s) administered may differ from the serotype(s) causative of the infection, disease or condition; and/or
(xix) an infection, disease or condition caused by any one or more serotypes of Streptococcus uberis by administering an immunogenic composition comprising one or more attenuated and/or killed streptococcal bacterial strains comprising a modification that attenuates, reduces or prevents functional expression of LytC (or a homologue thereof), wherein in the attenuated and/or killed streptococcal bacterial strains are one or more serotype(s) of Streptococcus uberis. The attenuated and/or killed Streptococcus uberis serotype(s) administered may differ from the serotype(s) causative of the infection, disease or condition.

In an embodiment, the methods are utilised to prevent or treat an infection, disease or condition caused by one or more serotypes of Streptococcus pneumoniae. The methods may comprise inducing an immune response in a subject against a plurality of different S. pneumoniae serotypes, by administering an immunogenic composition comprising one or more attenuated and/or killed streptococcal bacterial strain(s) comprising a modification that attenuates, reduces or prevents functional LytC expression to the subject. In an embodiment the methods comprise administering a single attenuated and/or killed (e.g., gamma-irradiated and/or X-irradiated) serotype of S. pneumoniae.

In an embodiment, the methods comprise preventing or treating an infection, disease or condition caused by one or more serotypes of Streptococcus pneumoniae. The methods comprise administering to a subject at least one serotype of attenuated and/or killed S. pneumoniae, and may induce an immune response in the subject against any one or more of S. pneumoniae serotypes 1, 2, 3, 4, 5, 6A, 6B, 6C, 6D, 7A, 7B, 7C, 7F, 8, 9A, 9L, 9N, 9V, 10A, 10B, 10C, 10F, 11A, 11B, 11C, 11D, 11F, 12A, 12B, 12F, 13, 14, 15A, 15B, 15C, 15F, 16A, 16F, 17A, 17F, 18A, 18B, 18C, 18F, 19A, 19B, 19C, 19F, 20, 21, 22A, 22F, 23A, 23B, 23F, 24A, 24B, 24C, 24F, 25A, 25F, 27, 28A, 28F, 29, 31, 32A, 32F, 33A, 33B, 33C, 33D, 33F, 34, 35A, 35B, 35C, 35F, 36A, 36B, 37, 38, 39, 40, 41A, 41F, 42, 43, 44, 45, 46, 47A, 47F, and/or 48. In an embodiment, the methods may induce an immune response in the subject against any one or more of S. pneumoniae serotypes 1, 2, 3, 4, 5, 6A, 6B, 7F, 8, 9N, 9V, 10A, 11A, 12F, 14, 15B, 17F, 18C, 19A, 19F, 20, 22F, 23F, and 33F. However, it is to be understood that the compositions of the present disclosure may induce an immune response against strains of streptococcal bacteria that are not specifically listed herein. For example, the compositions of the present disclosure may induce an immune response against emerging strains of S. pneumoniae, which have not yet been allocated a serotype number.

The attenuated and/or killed S. pneumoniae serotype(s) administered may differ from the serotype(s) causative of the infection, disease or condition. In an embodiment, the attenuated and/or killed streptococcal bacterial strain is derived from a different strain, serotype or species as the at least one target streptococcal species and/or serotype thereof. Accordingly, in an embodiment, the immune response induced is cross-protective.

The disease or condition may be any that is caused by infection of the particular species or serotype of streptococcal bacteria. By way of non-limiting example only, the disease or condition may be any one or more of: pneumonia, ear infection, earache, middle ear infection, otitis media, sinusitis, meningitis, conjunctivitis, bacteraemia, septicaemia, a joint infection, a bone infection, septic arthritis, osteomyelitis, a soft tissue infection, cellulitis, myositis, periorbital cellulitis, an abscess, peritonitis, a cardiac infection, endocarditis, and pericarditis.

The subject may be any animal of economic, social or research importance including bovine, equine, ovine, primate, avian and rodent species. Accordingly, the subject may be a mammal such as, for example, a human or a non-human mammal (e.g., a pig, cat, dog, cow, horse, or sheep). The subject may be a laboratory animal (e.g., a rodent such as a mouse, rat, or guinea pig; a rabbit, and the like), a bird (e.g., poultry), a fish or a crustacean.

The attenuated and/or killed streptococcal bacteria and/or attenuated and/or killed derivatives thereof may be administered to the subject by any suitable route including, for example, parenteral (e.g., intradermal, intravenous, intraspinal, intraperitoneal, subcutaneous or intramuscular), oral, topical, or mucosal routes (e.g., intranasal). In an embodiment, administration is by the mucosal route. For example, the administration may be intranasal.

Without being limited to specific mechanism(s) of action, the methods may induce an immune response in the subject comprising one or more of the following: (i) production of antibodies that bind specifically to antigen(s) of the streptococcal bacteria causative of the infection, disease or condition;
(ii) CD4+T lymphocyte responses specific for antigen(s) of the streptococcal bacteria causative of the infection, disease or condition; and/or
(iii) CD8+T lymphocyte responses specific for antigen(s) of the streptococcal bacteria causative of the infection, disease or condition.

In an embodiment, the methods may induce an immune response in the subject that may be Interleukin-17A (IL-17A) dependent, IL-17A independent, and/or that comprises activation of the innate immune system including the production of cytokines (e.g., IFN-γ) and/or activation of Toll-like receptors (e.g., TLR-3). This may assist in reducing the activation threshold for B cells and/or enhancing the quality or quantity of antibody responses against antigens of interest.

By way of non-limiting example only, an immune response induced or enhanced in a subject by the method may be increased by at least about 10%, at least about 20%, at least about 25%, at least about 50%, at least about 75%, at least about 90%, at least about two-fold, at least about five-fold, at least about ten-fold, at least about twenty-fold, at least about fifty-fold, or at least about 100-fold, when compared to a suitable control. The suitable control may, for example, be a measurement of the same immune response prior to performing the method under otherwise similar, substantially identical, or identical conditions.

Methods for detecting and quantifying immune response are well known to those of ordinary skill in the field and include, for example, solid-phase heterogeneous assays (e.g., enzyme-linked immunosorbent assay), solution phase assays (e.g., electrochemiluminescence assay), amplified luminescent proximity homogeneous assays, flow cytometry, intracellular cytokine staining, functional T-cell assays, functional B-cell assays, functional monocyte-macrophage assays, dendritic and reticular endothelial cell assays, measurement of natural killer cell responses, oxidative burst assays, cytotoxic specific cell lysis assays, pentamer binding assays, and phagocytosis and apoptosis evaluation.

### Immunogenic Composition and Vaccine Composition Formulations

The attenuated and/or killed streptococcal bacterial strain comprising a modification that attenuates, reduces or prevents functional expression of LytC (or a homologue thereof) described herein may be incorporated into pharmaceutical compositions. The compositions can stimulate an immune response against pathogenic organisms capable of establishing infection in a host that may culminate in a disease or condition. Accordingly, the compositions may be immunogenic compositions or vaccine compositions, including preventative vaccines (i.e., vaccines administered for the purpose of preventing infections and/or diseases/conditions) and therapeutic vaccines (i.e., vaccines administered for the purpose of treating infections and/or diseases/conditions). A vaccine of the present disclosure may therefore be administered to a recipient for prophylactic, ameliorative, palliative, or therapeutic purposes. It will be understood that all such compositions are collectively encompassed by reference herein to “compositions the disclosure” or a “composition of the disclosure”. It will also be understood that vaccine compositions fall within the scope of immunogenic compositions of the disclosure.

Non-limiting examples of suitable streptococcal bacteria suitable for incorporation into compositions of the disclosure are described above in the subsections entitled “Streptococcal bacterial strains” and “Streptococcal bacterial derivatives”.

In an embodiment, the streptococcal bacteria and their derivatives of the compositions are attenuated or inactivated by photon-radiation (e.g., gamma-radiation and/or X-radiation). The photon-radiation may be applied to the streptococcal bacterial strain of the present disclosure before, during or after combining them with other reagent(s) to provide an immunogenic composition of vaccine composition formulation.

### Formulations

Compositions of the disclosure may be prepared using methods known to those of ordinary skill in the art. Non-limiting examples of suitable methods are described in Gennaro et al. (Eds), (1990), “Remington's Pharmaceutical Sciences”, Mack Publishing Co., Easton, Pennsylvania, USA, and methods for vaccine preparation are generally described in Voller et al., (1978), “New Trends and Developments in Vaccines”, University Park Press, Baltimore, Maryland, USA.

The compositions may comprise a pharmaceutically acceptable carrier, excipient, diluent and/or adjuvant. “Pharmaceutically acceptable” carriers, excipients, diluents and/or adjuvants as contemplated herein are substances which do not produce adverse reaction(s) when administered to a particular recipient such as a human or non-human animal. Pharmaceutically acceptable carriers, excipients, diluents and adjuvants are generally also compatible with other ingredients of the vaccine. Non-limiting examples of suitable excipients, diluents, and carriers can be found in the “Handbook of Pharmaceutical Excipients” 4th Edition, (2003) Rowe et al. (Eds), The Pharmaceutical Press, London, American Pharmaceutical Association, Washington.

Non-limiting examples of pharmaceutically acceptable carriers, excipients or diluents include demineralised or distilled water; saline solution; vegetable based oils such as peanut oil, safflower oil, olive oil, cottonseed oil, maize oil, sesame oils, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

Compositions of the present disclosure may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, or in a form suitable for parenteral administration, that is, intradermal, subcutaneous, intramuscular or intravenous injection.

Solid forms of the compositions for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms of the compositions for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions comprising the compositions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

For preparation of the compositions as injectable solutions or suspensions, non-toxic parenterally acceptable diluents or carriers may be used such as Ringer's solution, isotonic saline, phosphate buffered saline, ethanol and 1,2 propylene glycol.

Vaccine emulsions for oral administration may further comprise one or more emulsifying agents. Suitable emulsifying agents include dispersing agents as exemplified above or natural gums such as guar gum, gum acacia or gum tragacanth.

Topical formulations of the compositions comprise an active ingredient(s) (e.g., attenuated and/or killed streptococcal bacteria and/or derivatives thereof) together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

When formulated as drops, the compositions may comprise sterile aqueous or oily solutions or suspensions. These may be prepared by dissolving the active ingredient in an aqueous solution of a bactericidal and/or fungicidal agent and/or any other suitable preservative, and optionally including a surface-active agent. The resulting solution may then be clarified by filtration, transferred to a suitable container and sterilised. For example, sterilisation may be achieved by filtration followed by transfer to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

When formulated as lotions, the compositions include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturizer such as glycerol, or oil such as castor oil or arachis oil.

When formulated as creams, ointments or pastes, the compositions may be semi-solid formulations of the active ingredient for external application. They may be made by mixing the active ingredient in finely divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols.

The compositions may include any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq.

### Adjuvants

Adjuvant(s) may be included in compositions of the disclosure, although experimental data provided herein demonstrates that attenuated and/or killed streptococcal bacteria and their derivatives can induce immunity without requiring such adjuvants.

Accordingly, compositions of the disclosure may or may not comprise an adjuvant.

In general, adjuvant activity in the context of a vaccine composition includes, but is not limited to, the ability to enhance the immune response (quantitatively or qualitatively) induced by immunogenic components in the vaccine (e.g., attenuated and/or killed streptococcal bacteria and/or derivatives thereof). This may reduce the dose or level of the immunogenic components required to produce an immune response and/or reduce the number or the frequency of immunisations required to produce the desired immune response.

Preferably, an adjuvant will enhance the immune response induced and/or enhanced by component(s) of the vaccine thereby improving protective efficacy. Preferably, the adjuvant will enable the induction of protective immunity utilising a lower dose of other active component(s) (e.g., attenuated and/or killed streptococcal bacteria and/or derivatives thereof).

Non-limiting examples of adjuvants suitable for inclusion in compositions of the disclosure and methods for their preparation are described in “Vaccine Adjuvants: Preparation Methods and Research Protocols (Methods in Molecular Medicine)”, (2000), Ohagan (Ed), Humana Press Inc. Any suitable adjuvant may be included in a vaccine of the disclosure.

Specific examples of such adjuvants include, but are not limited to, aluminium hydroxide; polypeptide adjuvants including interferons, interleukins, and other cytokines; AMPHIGEN, oil-in-water and water-in-oil emulsions; and saponins such as QuilA.

For example, an aluminium-based adjuvant may be utilised. Suitable aluminium-based adjuvants include, but are not limited to, aluminium hydroxide, aluminium phosphate and combinations thereof. Other specific examples of aluminium-based adjuvants that may be utilised are described in European Patent No. 1216053 and U.S. Pat. No. 6,372,223.

Oil in water emulsions may be utilised as adjuvants in compositions of the disclosure. Oil in water emulsions are well known in the art. In general, the oil in water emulsion will comprise a metabolisable oil, for example, a fish oil, a vegetable oil, or a synthetic oil. Examples of suitable oil in water emulsions include those described in European Patent No. 0399843, U.S. Pat. No. 7,029,678 and PCT Publication No. WO 2007/006939. The oil in water emulsion may be utilised in combination with other adjuvants and/or immunostimulants.

Non-limiting examples of other suitable adjuvants include immunostimulants such as granulocyte-macrophage colony-stimulating factor (GM-CSF), monophosphoryl lipid A (MPL), cholera toxin (CT) or its constituent subunit, heat labile enterotoxin (LT) or its constituent subunit, toll-like receptor ligand adjuvants such as lipopolysaccharide (LPS) and derivatives thereof (e.g., monophosphoryl lipid A and 3-Deacylated monophosphoryl lipid A), muramyl dipeptide (MDP), Toll-like receptor (TLR) agonists (e.g., TLR-2, TLR-3 agonists) and F protein of Respiratory Syncytial Virus (RSV).

Adjuvants in compositions of the disclosure may typically include emollients, emulsifiers, thickening agents, preservatives, bactericides and buffering agents. Another type of “self-adjuvant” is provided by the conjugation of immunogenic peptides to lipids such as the water soluble lipopeptides Pam3Cys or its dipalmitoyl derivative Pam2Cys.

Such adjuvants have the advantage of accompanying and immunogenic component into the antigen presenting cell (such as dendritic cells) and thus producing enhanced antigen presentation and activation of the cell at the same time (see, for example, Brown and Jackson, (2005), “Lipid based self adjuvanting vaccines”, Current Drug Delivery, 23:83).

Suitable adjuvants are commercially available such as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminium salts such as aluminium hydroxide gel (alum) or aluminium phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

In certain embodiments, an adjuvant included in a vaccine of the disclosure may induce an immune response predominantly of the Th1 type. Suitable adjuvants for use in eliciting a predominantly Th1-type response include, for example, a combination of monophosphoryl lipid A, preferably 3-de-O-acylated monophosphoryl lipid A (3D-MPL) together with an aluminium salt. For example, the composition or vaccine may be formulated with adjuvant AS04 containing aluminium hydroxide (alum) and 3-O-deacylated monophosphorylated lipid A (MPL) such as described in Thoelen et al. (2001), “A prophylactic hepatitis B vaccine with a novel adjuvant system”, Vaccine, 19:2400-2403. Other known adjuvants, which preferentially induce a Th1 type immune response, include CpG containing oligonucleotides. The oligonucleotides are characterised in that the CpG dinucleotide is unmethylated. Such oligonucleotides are known to those of ordinary skill in the field and are described, for example, in PCT Publication No. WO 1996/02555.

Immunostimulatory DNA sequences are also described, for example, in Sato et al., (1996), “Immunostimulatory DNA sequences necessary for effective intradermal gene immunization”, Science, 273:352-354.

Another example of an adjuvant is a saponin, preferably QS21 (Aquila Biopharmaceuticals Inc., Framingham, Mass.), which may be used alone or in combination with other adjuvants. For example, an enhanced adjuvant system may be utilised involving the combination of a monophosphoryl lipid A and saponin derivative, such as the combination of QS21 and 3D-MPL as described in PCT Publication No. WO 1994/00153, or a less reactogenic composition where the QS21 is quenched with cholesterol, as described in PCT publication No. WO 1996/33739. Other alternative formulations comprise an oil-in-water emulsion and tocopherol. An adjuvant formulation involving QS21, 3D-MPL and tocopherol in an oil-in-water emulsion is described in PCT Publication No. WO 1995/17210. An adjuvant included in a composition of the disclosure may include a formulation involving QS21, 3D-MPL and tocopherol in an oil in water emulsion such as described in PCT publication No. WO 1995/17210. In one embodiment a composition of the disclosure comprises the adjuvant Montanide ISA720 (M-ISA-720; Seppic, Fairfield, N.J.), an adjuvant based on a natural metabolisable oil.

Preferably, the adjuvant is a mucosal adjuvant effective in enhancing mucosal immunity and/or systemic immunity to immunogenic components administered via the mucosal route. Mucosal adjuvants may be broadly classified as those that facilitate vaccine delivery (e.g., liposomes, cochleates, live-attenuated vectors, poly D,L-lactide-co-glycolide or PLGA, chitans, DNA vaccines, mucoadhesives) to enhance the induction of protective immunity induced by other immunogenic components of the vaccine, and those having an immunostimulatory role (e.g., innate immunity associated toxin-based, cytokine-based etc.). Without limitation to a particular mechanism, it is postulated that the advantageous effects of mucosal adjuvants partially derive from an ability to assist the passage of immunogenic components in the vaccine across the mucosal barrier. Upon traversing the mucosal barrier, the mucosal adjuvant may enhance immunity, for example, by complement activation, the induction of cytokines, stimulation of antibody production or antibody type switching, stimulating antigen presenting cells, and/or influencing HLA or MHC class I and/or class II expression.

### Routes of Administration

Compositions of the disclosure may be administered to a recipient by standard routes, including, but not limited to, parenteral (e.g., intradermal, intravenous, intraspinal, intraperitoneal, subcutaneous or intramuscular), oral, topical, or mucosal routes (e.g., intranasal).

For example, the compositions may be administered by a mucosal route. Non-limiting examples of acceptable routes of mucosal vaccine administration including intranasal, ocular, buccal, genital tract (vaginal), rectal, intratracheal, skin, and the gastrointestinal tract.

In an embodiment, compositions of the disclosure are administered by the intranasal route. Without limitation to theory or particular mode(s) of action, intranasal administration of the compositions may be advantageous for enhancing immunity against certain streptococcal infections in which bacteria infect the host via mucosal surfaces of the upper and/or lower respiratory tracts. In addition, mucosal vaccination (e.g., intranasal vaccination) may induce mucosal immunity not only in the respiratory tracts but also in distant mucosal sites including the genital mucosa.

Intranasal compositions of the disclosure can be formulated, for example, in liquid form as nose drops, spray, or suitable for inhalation, as powder, as cream, or as emulsion.

Nebulised or aerosolised intranasal vaccines may also be utilised. Administration of compositions to mucosa of the upper and/or lower respiratory tract via inhalation of mists, powders, or sprays, or by intranasal administration of nose drops, swabs, powders, sprays, mists, aerosols, and the like is also contemplated.

In one embodiment, the compositions for intranasal administration are provided in a freeze-dried powder form capable of re-constitution immediately prior to use. Powder vaccine formulations of compositions of the present disclosure provide a means of overcoming refrigerated storage and distribution requirements associated with liquid-based vaccine stability and delivery. Dry powder formulations offer the advantage of being more stable and also do not support microbial growth.

The freeze-dried compositions may induce levels of cross-protective immunity similar to that of non-freeze-dried compositions. The compositions may be freeze-dried using any suitable technique known in the art. For example, liquid preparations of attenuated and/or killed streptococcal bacteria and/or derivatives thereof may be frozen in a dry ice—isopropanol slurry and lyophilized in a freeze Dryer (e.g., Virtis Model 10-324 Bench, Gardiner, NY) for a suitable time period (e.g., 24 hours).

In one embodiment, a dry powder nasal vaccine of the disclosure is produced by generating spray-freeze-drying (SFD) particles (see, for example, Costantino et al., (2002), “Protein spray freeze drying. 2. Effect of formulation variables on particle size and stability”, J Pharm Sci., 91:388-395; Costantino, et al., (2000), “Protein spray-freeze drying. Effect of atomization conditions on particle size and stability”, Pharm Res., 17:1374-1383; Maa et al., (1999), “Protein inhalation powders: spray drying vs spray freeze drying”, Pharm Res, 16:249-254; Carrasquillo et al., (2001); “Non-aqueous encapsulation of excipient-stabilized spray-freeze dried BSA into poly(lactide-co-glycolide) microspheres results in release of native protein”, J Control Release, 76:199-208; Carrasquillo et al., (2001), “Reduction of structural perturbations in bovine serum albumin by non-aqueous microencapsulation”, J Pharm Pharmacol., 53:115-120; and U.S. Pat. No. 6,569,458).

Preferred devices for intranasal administration of the compositions are nasal spray devices (e.g., devices available commercially from Pfeiffer GmBH, Valois and Becton Dickinson). Non-limiting examples of suitable devices are described, for example, in Bommer, (1999), “Advances in Nasal drug delivery Technology”, Pharmaceutical Technology Europe, p 26-33. Intranasal devices may produce droplets in the range 1 to 500 m. Preferably, only a small percentage of droplets (e.g., <5%) are below 10 m to minimise the chance of inhalation. Intranasal devices may be capable of bi-dose delivery, that is, the delivery of two subdoses of a single vaccination dose, one sub-dose to each nostril.

A composition of the disclosure may be administered to a recipient in isolation or in combination with other additional therapeutic agent(s). In embodiments where the vaccine is administered with therapeutic agent(s), the administration may be simultaneous or sequential (i.e., vaccine administration followed by administration of the agent(s) or vice versa). Thus, where a vaccine of the disclosure is administered to a subject in conjunction with another agent, both may be administered in a single composition at the same time, in separate compositions at the same time, or separately at different times.

### Dosages

In general, compositions of the disclosure are administered in a manner compatible with the route of administration and physical characteristics of the recipient (including health status) and in such a way that it is elicits the desired effect(s) (i.e., therapeutically effective, immunogenic and/or protective).

For example, the appropriate dosage of a given vaccine may depend on a variety of factors including, but not limited to, a subject's physical characteristics (e.g., age, weight, sex), whether the compound is being used as single agent or adjuvant therapy, the progression (i.e., pathological state) of a given streptococcal infection, and other factors that may be recognized by one skilled in the art. Various general considerations that may be considered when determining an appropriate dosage of a given vaccine of the disclosure are described, for example, in Gennaro et al. (Eds), (1990), “Remington's Pharmaceutical Sciences”, Mack Publishing Co., Easton, Pennsylvania, USA; and Gilman et al., (Eds), (1990), “Goodman And Gilman's: The Pharmacological Bases of Therapeutics”, Pergamon Press.

In general, compositions of the disclosure may be administered to a patient in an amount of from about 5 micrograms to about 5 mg of active component(s) (i.e., attenuated and/or killed streptococcal bacteria and/or derivatives thereof). Dosage in an amount of from about 50 micrograms to about 500 micrograms is especially preferred.

One skilled in the art would be able, by routine experimentation, to determine an effective, non-toxic amount of attenuated and/or killed streptococcal bacteria or derivatives thereof to include in a vaccine of the disclosure for the desired therapeutic outcome.

Generally, an effective dosage is expected to be in the range of about 0.0001 mg to about 1000 mg of active component(s) (i.e., attenuated and/or killed streptococcal bacteria or derivatives thereof) per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m² of active component(s) (i.e., attenuated and/or killed streptococcal bacteria or derivatives thereof). Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Typically, in therapeutic applications, the treatment would be for the duration of the infection, disease state or condition. Further, it will be apparent to one of ordinary skill in the art that the optimal quantity and spacing of individual dosages will be determined by the nature and extent of the infection, disease state or condition being treated, the form, route and site of administration, and the nature of the particular individual being treated. Also, such optimum conditions can be determined by conventional techniques.

In many instances, it will be desirable to have several or multiple administrations of a vaccine of present disclosure. For example, compositions of the disclosure may be administered 1, 2, 3, 4, 5, 6, 7, 8, 9, 10, or more times. The administrations may be from about one to about twelve-week intervals, six-month intervals, twelve-month intervals, etc., and in certain embodiments from about one to about four-week intervals. Periodic re-administration may be desirable in the case of recurrent exposure to a particular pathogen targeted by a vaccine of the disclosure.

It will also be apparent to one of ordinary skill in the art that the optimal course of treatment can be ascertained using conventional course of treatment determination tests.

The methods described herein may comprise administering a priming dose of a vaccine of the disclosure. The priming dose may be followed by a booster dose. The booster may be for the purpose of revaccination. In various embodiments, the vaccine is administered at least once, twice, three times or more. Compositions of the disclosure may be administered to naïve recipients, being individuals seronegative for particular target strain(s) of streptococcal bacteria. Alternatively, the compositions may be administered to primed recipients, being individuals seropositive for particular target strain(s) of streptococcal bacteria.

It will be appreciated by persons of ordinary skill in the art that numerous variations and/or modifications can be made to the present disclosure as disclosed in the specific embodiments without departing from the spirit or scope of the present disclosure as broadly described. The present embodiments are, therefore, to be considered in all respects as illustrative and not restrictive.

### Examples

### Nucleic Acid and Amino Acid Sequences

Nucleic acid sequences relevant to the present examples are listed in Table One.

TABLE One
Polynucleotide and amino acid sequences

| SEQ | | |
|---|---|---|
| ID | | |
| No. | Description | Sequence |
| 1 | Rx1 DNA | TTTGGTCAAGAAGTCGCAGACAAAGTTCGTGTTCAATACGGTGGTTCTGT |
| | sequence of | TAAACCTGAAAATGTTGCTTCATACATGGCTTGCCCAGACGTTGACGGTG |
| | lytC (bold) | CCCTTGTAGGTGGTGCGTCACTTGAAGCTGAAAGCTTCTTGGCTTTGCTT |
| | including | GACTTTGTAAAATAATCAGTAAGTAGCAAAAGCTAGGTGGAACAGCATTC |
| | flanking | AGATGTCTGTTACATTTTTTATAGGAGAGGAAAGATTGAAAATAAAAATT |
| | regions | GGATTAGCAAGTATCTGTTTACTAGGCTTGGCAACTAGTCATGTCGCTGC |
| | | AAATGAAACTGAAGTAGCAAAAACTTCGCAGGATACAACGACAGCTTCAA |
| | | GTAGTTCAGAGCAAAATCAGTCTTCTAATAAAACGCAAACGAGCGCAGAA |
| | | GTACAGACTAATGCTGCTGCCTACTGGGATGGGGATTATTATGTAAAGGA |
| | | TGATGGTTCTAAAGCTCAAAGTGAATGGATTTTTGACAACTACTATAAGG |
| | | CTTGGTTTTATATTAATTCAGATGGTCGTTACTCGCAGAATGAATGGCAT |
| | | GGAAATTACTACCTGAAATCAGGTGGATATATGGCCCAAAACGAGTGGAT |
| | | CTATGACAGTAATTACAAGAGTTGGTTTTATCTCAAGTCAGATGGGGCTT |
| | | ATGCTCATCAAGAATGGCAATTGATTGGAAATAAGTGGTACTACTTCAAG |
| | | AAGTGGGGTTACATGGCTAAAAGCCAATGGCAAGGAAGTTATTTCTTGAA |
| | | TGGTCAAGGAGCTATGATACAAAATGAATGGCTCTATGATCCAGCTTATT |
| | | CTGCTTATTTTTATCTAAAATCCGATGGAACTTATGCTAACCAAGAGTGG |
| | | CAAAAAGTGGGCGGCAAATGGTACTATTTCAAGAAGTGGGGCTATATGGC |
| | | TCGGAATGAGTGGCAAGGCAACTACTATTTGACTGGAAGTGGTGCCATGG |
| | | CGACTGACGAAGTGATTATGGATGGTGCTCGCTATATCTTTGCGGCCTCT |
| | | GGTGAGCTCAAAGAAAAGAAAGATTTGAATGTCGGCTGGGTTCACAGAGA |
| | | TGGTAAACGCTATTTCTTTAATAATAGAGAAGAACAAGTGGGAACCGAAC |
| | | ATGCTAAGAAAATCATTGATATTAGTGAGCACAATGGTCGTATCAATGAT |
| | | TGGAAAAAGGTTATTGATGAGAACGAGGTGGATGGTGTCATTGTTCGTCT |
| | | AGGTTATAGCGGTAAAGAAGACAAGGAATTGGCGCATAACATTAAGGAGT |
| | | TAAACCGTCTGGGAATTCCTTATGGTGTCTATCTCTATACCTATGCTGAA |
| | | AATGAGACCGATGCTGAGAATGACGCTAAACAGACCATTGAACTTATAAA |
| | | GAAATACAATATGAACCTGTCTTACCCTATCTATTATGATGTTGAGAACT |
| | | GGGAATATGTAAATAAGAGCAAGAGAGCTCCAAGTGATACAGACACTTGG |
| | | GTTAAAATCATCAACAAGTACATGGACACGATGAAGCAGGCGGGTTATCA |
| | | AAATGTATATGTCTATAGCTATCGTAGTTTATTACAGACGCGTTTAAAAC |
| | | ACCCAGATATTTTAAAACATGTAAACTGGGTAGCGGCCTATACGAATGCT |
| | | TTAGAATGGGAAAACCCTTATTATTCAGGAGAAAAAGGTTGGCAATATAC |
| | | CTCTTCTGAATACATGAAAGGAATCCAAGGGCGCGTAGATGTCAGCGTTT |
| | | GGTATTAAGCGATGATTTGAAAGAGGGATGTGATAGTAGCACCCTCTTTT |
| | | TCTTTGTTTTATGATAGTTCATCCTCGAGTAAATTCAAGATCTTGCTCGG |
| | | AAATGAAGCTTATATAGTAGATTGAATATAGACAAATACCTTGTGATTGG |
| | | TAAAACATTTTAGAAATTCATTTACCTTTCCTAATCGACTTGG |
| 2 | lytC_F1 | CTGGGCTTTATCTGCGCTATG |
| | Primer | |
| 3 | lytC_R1 | CCATACTTTTGTGCCCGTCTTTCCTCTCCTATAAAAAATGTAACAGA |
| | Primer | |
| 4 | lytC_F2 | GAATTGTTTTAGTACCTAGAATTCACCGCGATGATTTGAAAGAGGGATG |
| | Primer | |
| 5 | lytC_R2 | ACTTCCTCAACCAATTCTTCTCG |
| | Primer | |
| 6 | kanamycinR_ | ACGGGCACAAAAGTATGG |
| | F Primer | |
| 7 | kanamycinR_ | GGTGAATTCTAGGTACTAAAACAATTC |
| | R Primer | |
| 8 | lytC_MD_R | CATCCCTCTTTCAAATCATCGCTCTTTCCTCTCCTATAAAAAATGTAACA |
| | | GA |
| 9 | lytC_MD_F | TCTGTTACATTTTTTATAGGAGAGGAAAGAGCGATGATTTGAAAGAGGGA |
| | | TG |
| 10 | psaA_seq_F | GTGTCGGAACTATCCTGATTGTAG |
| 11 | psaA_seq_R | GAGTTGAGCAGATGCCTGTATC |
| 12 | lytA_seq_F | GTTTGGTGTTGAAAATAGTTTAACAG |
| 13 | lytA_seq_R | CGACTCGGTTTACATTGTC |
| 14 | lytC_seq_F | TTTGGTCAAGAAGTCGCAGAC |
| 15 | lytC_seq_R | CCAAGTCGATTAGGAAAGGTAAATG |
| 16 | PdT_seq_F | AGATTTTGGACAATACAGAAGTG |
| 17 | PdT_seq_R | CCTGTTTGATAGACTCATTTTAAC |
| 18 | GPN-003 | TTTGGTCAAGAAGTCGCAGACAAAGTTCGTGTTCAATACGGTGGTTCTGT |
| | DNA | TAAACCTGAAAATGTTGCTTCATACATGGCTTGCCCAGACGTTGACGGTG |
| | sequence of | CCCTTGTAGGTGGTGCGTCACTTGAAGCTGAAAGCTTCTTGGCTTTGCTT |
| | LytC region | GACTTTGTAAAATAATCAGTAAGTAGCAAAAGCTAGGTGGAACAGCATTC |
| | following | AGATGTCTGTTACATTTTTTATAGGAGAGGAAAGAGCGATGATTTGAAAG |
| | deletion of | AGGGATGTGATAGTAGCACCCTCTTTTTCTTTGTTTTATGATAGTTCATC |
| | lytC (i.e., | CTCGAGTAAATTCAAGATCTTGCTCGGAAATGAAGCTTATATAGTAGATT |
| | region | GAATATAGACAAATACCTTGTGATTGGTAAAACATTTTAGAAATTCATTT |
| | originally | ACCTTTCCTAATCGACTTGG |
| | harbouring | |
| | lytC including | |
| | flanking | |
| | regions) | |
| 19 | GPN-003 | GATTCAAGAAGGCAGTCGCTTTACAGCAGATCATCCAGGCTTGCCGATTT |
| | DNA | CCTATACAACTTCTTTTTTACGTGACAATGTAGTTGCGACCTTTCAAAAC |
| | sequence of | AGTACAGACTATGTTGAGACTAAGGTTACAGCTTACAGAAACGGAGATTT |
| | the 3′ end of | ACTGCTGGATCATAGTGGTGCCTATGTTGCCCAATATTATATTACTTGGG |
| | the pdt | ATGAATTATCCTATAATCATCAAGGTAAGGAAGTCTTGACTCCTAAGGCT |
| | region | TGGGACAGAAATGGGCAGGATTTGACGGCTCACTTTACCACTAGTATTCC |
| | | TTTAAAAGGGAATGTTCGTAATCTCTCTGTCAAAATTAGAGAGGGTACCG |
| | | GGCTTGCCTTCGAATGGTGGCGTACGGTTTATGAAAAAACCGATTTGCCA |
| | | CTAGTGCGTAAGCGGACGATTTCTATTTGGGGAACAACTCTCTATCCTCA |
| | | GGTAGAGGATAAGGTAGAAAATGACTAG |
| 20 | prsA_F | CGTGAGCCAATCACTTCAAA |
| 21 | prsA_R | CTCAAAATAATCTGCAATTA |
| 22 | gyrA_F | ACTGGTATCGCGGTTGGGAT |
| 23 | gyrA_R | ACCTGATTTCCCCATGACAA |
| 24 | Rx1 3′ ply | IQEGSRFTADHPGLPISYTTSFLRDNVVATFQNSTDYVETKVTAYRNGDL |
| | region. Bold | LLDHSGAYVAQYYITWDELSYDHQGKEVLTPKAWDRNGQDLTAHFTTSIP |
| | text indicates | LKGNVRNLSVKIRECTGLAWEWWRTVYEKTDLPLVRKRTISIWGTTLYPQ |
| | ply amino | VEDKVEND* |
| | acids that are | |
| | modified in | |
| | pdT. | |
| 25 | GPN-003 3′ | IQEGSRFTADHPGLPISYTTSFLRDNVVATFQNSTDYVETKVTAYRNGDL |
| | pdt region. | LLDHSGAYVAQYYITWDELSYNHQGKEVLTPKAWDRNGQDLTAHFTTSIP |
| | Bold text | LKGNVRNLSVKIREGTGLAFEWWRTVYEKTDLPLVRKRTISIWGTTLYPQ |
| | indicates | VEDKVEND* |
| | modified PdT | |
| | amino acids. | |
| 26 | Rx1 DNA | GATTCAAGAAGGCAGTCGCTTTACAGCAGATCATCCAGGCTTGCCGATTT |
| | sequence for | CCTATACAACTTCTTTTTTACGTGACAATGTAGTTGCGACCTTTCAAAAC |
| | the 3′ region | AGTACAGACTATGTTGAGACTAAGGTTACAGCTTACAGAAACGGAGATTT |
| | of the ply | ACTGCTGGATCATAGTGGTGCCTATGTTGCCCAATATTATATTACTTGGG |
| | gene | ATGAATTATCCTATAATCATCAAGGTAAGGAAGTCTTGACTCCTAAGGCT |
| | | TGGGACAGAAATGGGCAGGATTTGACGGCTCACTTTACCACTAGTATTCC |
| | | TTTAAAAGGGAATGTTCGTAATCTCTCTGTCAAAATTAGAGAGGGTACCG |
| | | GGCTTGCCTTCGAATGGTGGCGTACGGTTTATGAAAAAACCGATTTGCCA |
| | | CTAGTGCGTAAGCGGACGATTTCTATTTGGGGAACAACTCTCTATCCTCA |
| | | GGTAGAGGATAAGGTAGAAAATGACTAG |

### Example One: Construction of GPN-001ΔytC::kanR and GPN-002ΔlytC::kanR Streptococcal Vaccine Strains

Deletions of the lytC gene were engineered into GPN-001 and GPN-002, which are both unencapsulated vaccine strains derived from S. pneumoniae Rx1, as shown in Figure One. GPN-001 has a deletion of the autolysin gene lytA, and the pneumolysin gene (ply) has been replaced with a mutated ply gene encoding a toxoid version of Ply, designated PdT. GPN-002 is a derivative of the GPN-001 strain. GPN-002 additionally has a deletion of a gene (psaA) encoding the solute binding component of the manganese uptake system.

Deletion of lytC from GPN-001 and GPN-002 to Form GPN-001ΔlytC::kanR and GPN-002ΔlytC::kanR Strains

The lytC gene was removed from GPN-001 and GPN-0002 using conventional methods as presented in Figure One. The DNA sequence of the lytC gene, prior to deletion (including approximately 200 bp flanking regions), is shown in SEQ ID NO: 1.

Briefly, the lytC gene was deleted by replacement with a kanamycin resistance cassette. Oligonucleotide primers were designed to amplify by polymerase chain reaction (PCR) a region of approximately 2 kilobases (kb) directly upstream of the lytC gene using primers lytC_F1 (SEQ ID NO: 2) and lytC_R1 (SEQ ID NO: 3); and downstream of the lytC gene using primers (lytC_F2 (SEQ ID NO: 4) and lytC_R2 5′-(SEQ ID NO: 5). These primers additionally incorporated complementary regions to a kanamycin resistance cassette.

A kanamycin resistance cassette was amplified using primers kanamycinR_F (SEQ ID NO: 6) and kanamycinR_R (SEQ ID NO: 7). All PCRs were performed using the Phusion® High-Fidelity PCR Master Mix with HF Buffer (New England BioLabs) using standard parameters and oligonucleotide primers were purchased from Sigma/Merck. A linear fragment of these three products was generated by overlap extension PCR using primers lytC_F1 (SEQ ID NO: 2) and lytC_R2 (SEQ ID NO: 5) using standard parameters. The linear lytC::kanR fragment was transformed into the GPN-001 and GPN-002 strains using standard methods. Successful replacement of the lytC gene with a kanamycin resistance cassette was determined by assessment of capacity to grow on manganese- and kanamycin-supplemented blood agar plates, and size analysis by agarose gel electrophoresis following amplification of the region by PCR.

Transformants were confirmed by PCR amplification using oligonucleotide primer combinations designed to test the presence or absence and orientation of the kanamycin resistance cassette. For product A, primers lytC_F1 5 (SEQ ID NO: 2) and lytC_R2 (SEQ ID NO: 5) were expected to yield an approximately 5.6 kb product in parental strain GPN-001 and GPN-002 and an approximately 4.6 kb product GPN-001ΔlytC and GPN-002ΔlytC). Products B and C were amplified to confirm the presence and orientation of the kanamycin resistance cassette. Product B was amplified using primers lytC_F1 (SEQ ID NO: 2) and kanamycinR_R (SEQ ID NO: 7). Product C was amplified using primers kanamycinR_F (SEQ ID NO: 6) and lytC_R2 (SEQ ID NO: 5). PCR using the Product B and C primer sets was expected to yield no product in parental strains GPN-001 and GPN-002, but an approximately 2.5 kb product in the GPN-001ΔlytC::kanR and GPN-002ΔlytC::kanR strains.

### Results and Discussion

Successful incorporation of the kanamycin resistance cassette at the lytC region of GPN002 was confirmed by comparing PCR products of the GPN-002 parental strain and three clones of the GPN-002ΔlytC::kanR strain. Sizes of products A, B and C as described above were determined by gel electrophoresis with the expected reduction in size of product A for the GPN-002ΔlytC::kanR strain observed (approximately 4.6 kb) and expected approximately 2.5 kb sizes observed for products B and C in all clones (Figure Two).

The primers and methods used in this example could be used to generate ΔlytC::kanR mutations in other S. pneumoniae strains including both encapsulated and unencapsulated strains.

Upon confirmation of the successful integration of the kanamycin resistance cassette into the strains, further studies proceeded with GPN-002ΔlytC::kanR clone 1 strain.

### Example Two: Deletion of lytC Reduced Bacterial Lysis During Vaccine Production

Experiments were performed to investigate whether deletion of lytC in a vaccine strain improved vaccine stability during manufacture by preventing bacterial lysis in buffer.

The stability of a strain harbouring a deletion in lytC was assessed by measurements of the cell density over time at 25° C. The GPN-002 and GPN-002ΔlytC::kanR strains were grown to equivalent optical densities at 600 nm (approximately OD₆₀₀ 1.2) in a standard soy-based animal free growth medium supplemented with 5 μM MnSO₄. The bacteria were washed and resuspended in phosphate buffered saline (PBS) with 20% (w/v) glycerol. A volume of 100 μL of each sample or buffer only was transferred to wells of a 96-well flat-bottom plate. The OD₆₀₀ was measured at 15 min intervals at 25° C. in a spectrophotometer (BMG Labtech).

### Results and Discussion

Data are presented as the mean optical density at 600 nm at each time point +/− standard error of the mean over time (Figure Three) and as representative timepoints at time=0, 2, 4, 8 and 16 hrs (Figure Four). GPN-002ΔlytC::kanR maintained a higher optical density during incubation at 25° C. in PBS glycerol buffer compared with the GPN-002 parental strain.

This result shows that the deletion of lytC in GPN-002 resulted in enhanced stability and reduced lysis in Streptococcus pneumoniae.

Stability of other Streptococcal bacteria can be tested using the same method. On the basis of the results of this example, it is anticipated that Streptococcal bacteria derivatives having a lytC deletion have improved stability compared to a parental strain.

### Example Three: Construction and Testing of GPN-003 Harbouring Unmarked Deletion of lytC Gene

The antibiotic resistance cassette was removed from GPN-002ΔlytC::kanR to generate GPN-003, a vaccine strain more suitable for human use.

Deletion of the Kanamycin Resistance Cassette from GPN-002ΔlytC::kanR

The kanamycin resistance cassette introduced into GPN-002 in strain GPN-002ΔlytC::kanR was removed to generate an unmarked deletion of the lytC coding region. PCR was used to amplify regions of approximately 2 kb directly upstream of the lytC gene using primers lytC_F1 (SEQ ID NO: 2) and lytC_MD_R (SEQ ID NO: 8); and downstream of the lytC gene using primers lytC_MD_F (SEQ ID NO: 9) and lytC_R2 (SEQ ID NO: 5) to incorporate approximately 20-30 base pairs (bp) of complementary homology to the regions immediately upstream and downstream of the lytC region. A linear fragment of these two products was generated by overlap extension PCR using primers lytC_F1 (SEQ ID NO: 2) and lytC_R2 (SEQ ID NO: 5).

The linear markerless deletion fragment (lytC-) was transformed into the GPN-002ΔlytC::kanR resistant strain essentially as described above, except with an enrichment step following the addition of the linear DNA to facilitate selection of kanamycin-sensitive clones. Briefly, S. pneumoniae GPN-002ΔlytC::kanR competent cells were incubated with 100 ng/mL competence stimulating peptide 1 (CSP-1) for 15 minutes. The linear lytC-fragment was added to the transformation reaction and enrichment of the deletion strain was achieved by culturing in the presence of kanamycin, followed by ampicillin treatment at standard concentrations. Successful incorporation was determined by assessment of antibiotic resistance profile by patch testing on manganese-, kanamycin-, and unsupplemented blood agar plates, sequencing, and size analysis by agarose gel electrophoresis. The resulting strain, S. pneumoniae GPN-002ΔlytC, which harbours an unmarked lytC deletion was designated GPN-003.

### Results and Discussion

The genetic modifications present in GPN-003 relative to Rx1 were confirmed based on size (Figure Five) using the following oligonucleotide primers:
psaA: psaA_seq_F (SEQ ID NO: 10) and psaA_seq_R (SEQ ID NO: 11);
lytA: lytA_seq_F (SEQ ID NO: 12) and lytA_seq_R (SEQ ID NO: 13);
lytC: lytC_seq_F (SEQ ID NO: 14) and lytC_seq_R (SEQ ID NO: 15); and pdt: PdT_seq_F (SEQ ID NO: 16) and PdT_seq_R (SEQ ID NO: 17).

The sequence of the lytC deletion in GPN-003 was further confirmed by Sanger Sequencing (Australian Genome Reference Facility, Australia). The DNA sequence following deletion of the region originally harbouring lytC (including approximately 200 bp flanking regions) is shown in SEQ ID NO: 18. The sequence of the 3′ end of the pdt region of GPN-003 was confirmed by Sanger Sequencing (AGRF) and is shown in SEQ ID NO: 19 and with the protein translation in Figure Six (lower panel) and SEQ ID NO: 25. The sequence of the 3′ end of the Rx1 ply gene is shown in SEQ ID NO: 26 and the protein translation is shown in Figure Six (upper panel) and SEQ ID NO: 24 for reference. The confirmed sizes of the products and nature of modifications in GPN-003 relative to Rx1 are summarised in Table Two.

TABLE TWO
Summary of sizes of lytA, ply, psaA and lytC amplified
PCR products in Rx1 and GPN-003 strains

| Gene | | | |
|---|---|---|---|
| (Rx1 locus tag) | Size Rx1 | Size GPN-003 | Nature of mutation |
| lytA | Approximately | Approximately | Deletion |
| (KZH43_08650) | 1448 bp | 491 bp | |
| ply/PdT | Approximately | Approximately | Amino acid substitutions |
| (KZH43_08585) | 702 bp | 702 bp | (Asp₃₈₅→Asn, Cys₄₂₈→Gly and |
| | | | Trp₄₃₃→Phe) |
| psaA | Approximately | Approximately | Deletion |
| (KZH43_07260) | 1530 bp | 600 bp | |
| lytC | Approximately | Approximately | Deletion |
| (KZH43_06955) | 1893 bp | 387 bp | |

### Example Four: Stability of GPN-002 and GPN-003 Strains

The stability of the GPN-002 and GPN-003 strains was compared by measurements of the cell density over time at 25° C. The GPN-002 and GPN-003 strains were grown to equivalent optical densities at 600 nm in a standard soy-based animal free growth medium supplemented with 5 μM MnSO₄. The bacteria were washed and resuspended in phosphate buffered saline (PBS) with 20% (w/v) glycerol. A volume of 100 μL of each sample or buffer only was transferred to wells of a 96-well flat-bottom plate. The OD₆₀₀ was measured at 15 min intervals at 25° C. in a spectrophotometer (BMG Labtech).

### Results and Discussion

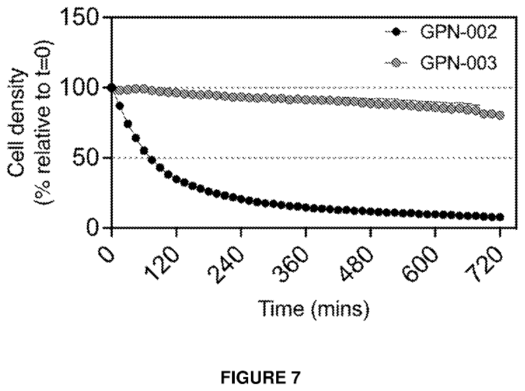

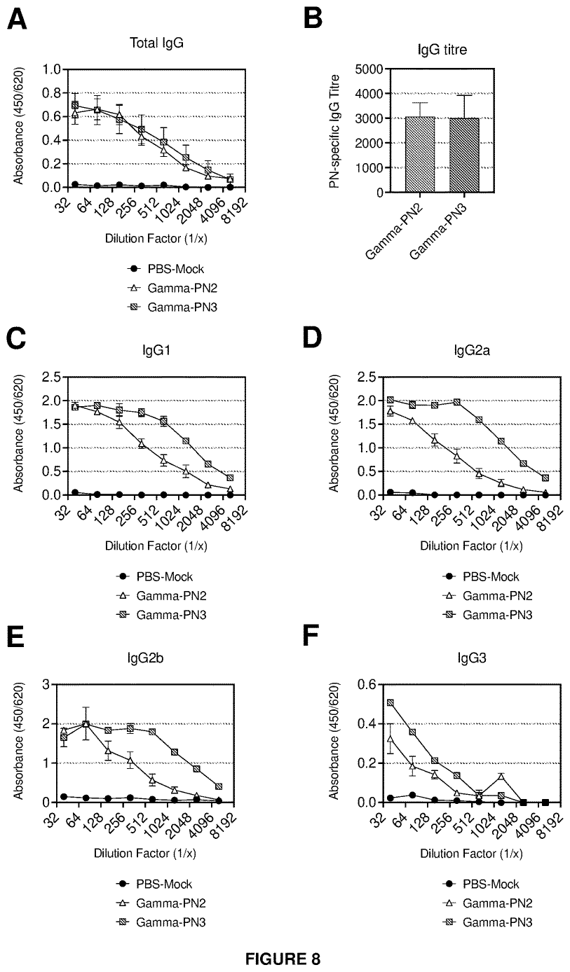

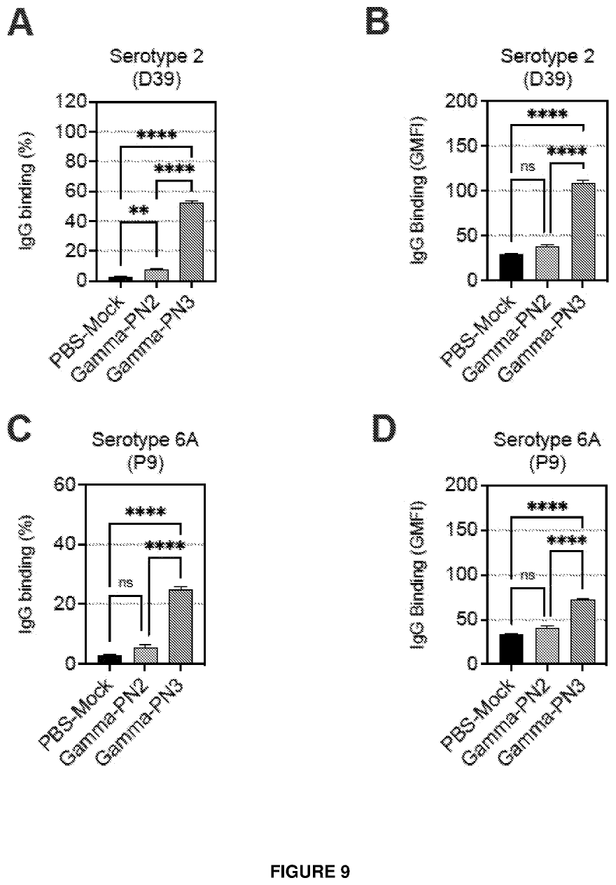

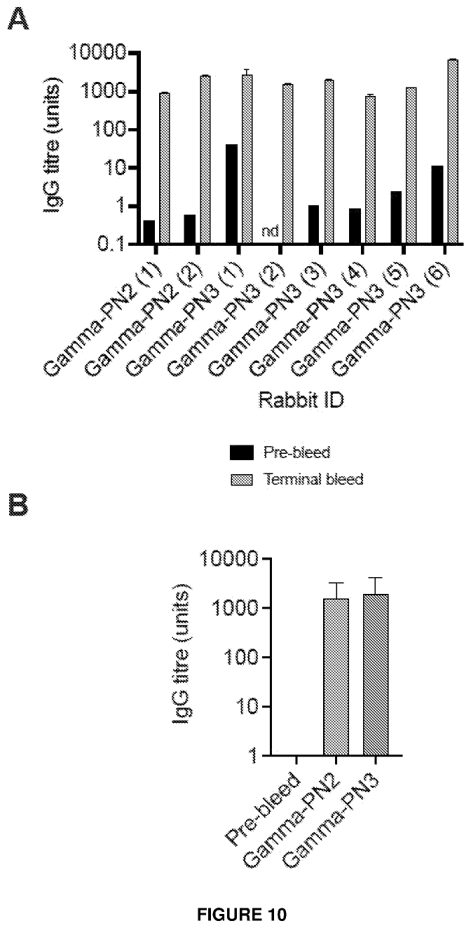

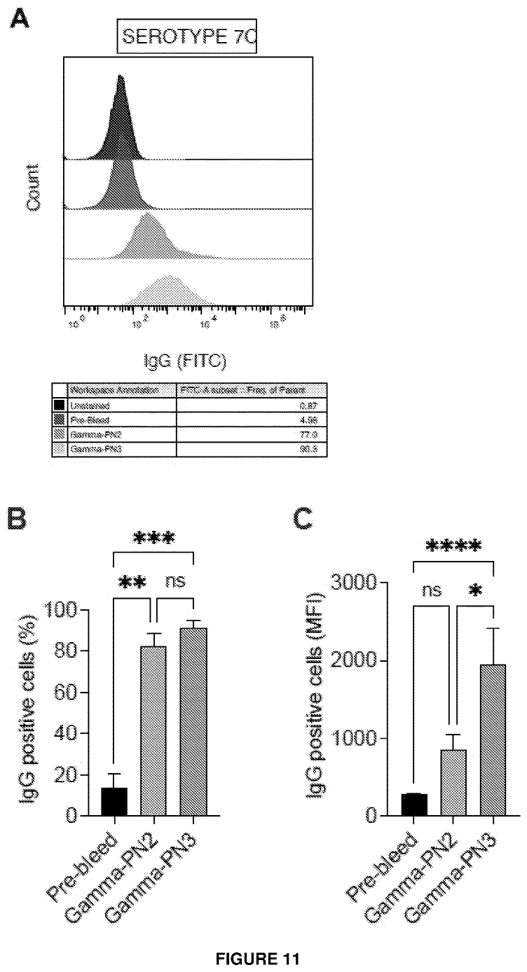

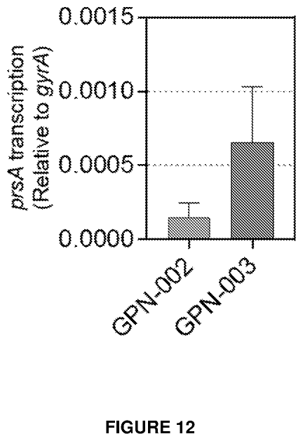

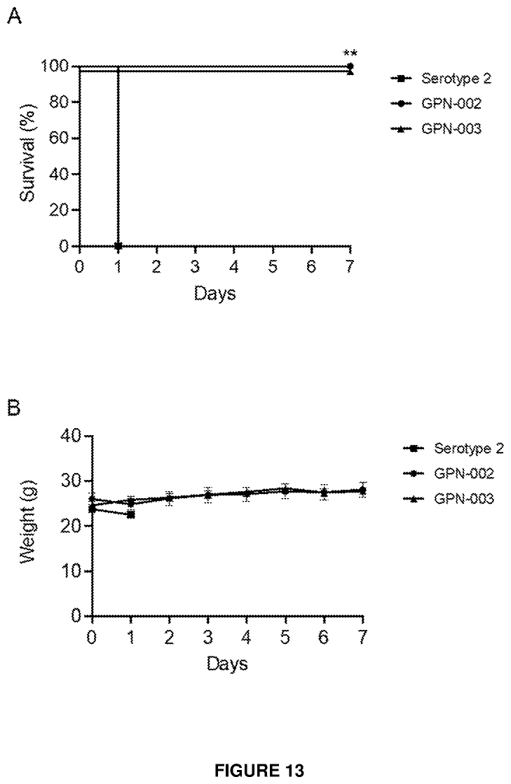

The percentage cell density was calculated relative to the starting optical density at 600 nm. Data are presented as the mean percentage relative to the input at each time point +/− standard error of the mean over time (FIG. 7).

GPN-003, which possesses an unmarked deletion of lytC compared to parental strain GPN-002, has enhanced stability and reduced lysis compared with the GPN-002. That is, removal of the lytC gene from the vaccine strain resulted in a reduction in bacterial lysis upon holding the bacteria for prolonged periods at room temperature. This demonstrates that the deletion of lytC in Streptococcus pneumoniae bacteria provides enhanced stability.

Stability of other streptococcal bacteria can be tested using the same method. On the basis of the results of this example, it is anticipated that streptococcal bacteria having a lytC deletion have improved stability compared to parental strain.

### Example Five: Analysis of Total IgG and Subclass IgG Responses from Mice Vaccinated with Gamma-Irradiated GPN-003 (Gamma-PN3) Preparation of Gamma-PN2 and Gamma-PN3 Vaccines

To assess whether a vaccine based on a gamma-irradiated GPN-003 strain induced similar antibody responses to strain containing a functional LytC protein, a vaccination experiment in an animal model was conducted. The GPN-002 and GPN-003 strains were cultured as described above. The bacteria were washed and resuspended in phosphate buffered saline (PBS) with 20% (w/v) glycerol and inactivated by exposure to 26 kGy of gamma radiation. The resulting vaccines were designated Gamma-PN2 and Gamma-PN3, respectively. The capacity of these vaccines to induce pneumococcal-specific antibody responses and the effect on IgG subclasses was investigated.

Vaccination of Mice with Gamma-PN2 and Gamma-PN3 Vaccines

Outbred Swiss mice were intramuscularly (I.M.) vaccinated with 50 g total protein of Gamma-PN2 and Gamma-PN3 three times, two weeks apart. Control mice were mock vaccinated with PBS with 20% (w/v) glycerol. Two weeks post final vaccination, serum was taken from all mice. Gamma-PN2 and Gamma-PN3 were administered in the absence of an adjuvant.

### Detection of IgG Responses Following Vaccination

Serum samples were tested for S. pneumoniae-specific IgG responses by direct Enzyme Linked Immunosorbent Assay (ELISA) using standard techniques. Rx1, the unencapsulated derivative of D39 (serotype 2) was used as the coating antigen.

### Results and Discussion

Figure Eight (A) shows pneumococcal-specific IgG responses as absorbance (450/620 nm), and Figure Eight (B) as titres. Data are presented as mean absorbance (450/620 nm)±SEM from individual or pooled serum samples (n=5 mice per group). The IgG1 subclass response is shown in Figure Eight (C), the IgG2a response in Figure Eight (D), IgG2b response is in Figure Eight (E), and IgG3 response is shown in Figure Eight (F), as determined by ELISA using Rx1 as the coating antigen. The ratio of these subclasses in each sample is presented in Table Three.

TABLE THREE
IgG subclass ratios induced in mice following
vaccination with Gamma-PN2 and Gamma-PN3

| IgG Subclass Ratio | Gamma-PN2 | Gamma-PN3 |
|---|---|---|
| IgG1:IgG2a | 3 (±1.59) | 1.10 (±0.27) |
| IgG1:IgG2b | 3.41 (±2.25) | 1.12 (±0.19) |
| IgG1:IgG3 | 8.33 (±3.52) | 7 (±1.15) |

Gamma-PN2 and Gamma-PN3 induced significant pneumococcal-specific IgG antibody responses after immunisation in mice compared to mock-vaccinated mice. Notably, Gamma-PN3-vaccinated mice had significantly greater titres of pneumococcal-specific IgG1, IgG2a, IgG2b, and IgG3 than Gamma-PN2-vaccinated mice.

This result surprisingly showed that deleting the lytC gene enhanced the antibody response. This was unexpected as LytC is considered to be an immunogen due to its surface location on streptococcal bacteria and its function as a virulence factor. For example, vaccination with purified LytC showed the potential of this protein as a protective immunogen, with immunised mice exhibiting high Immunoglobulin G (IgG) antibodies and enhanced protection against pneumococcal infection in mice (Wizemann et al., 2001; Corsini et al., 2021); and high titres of LytC-specific IgG antibodies have also been observed in serum samples from healthy volunteers in multiple studies, indicating the induction of anti-LytC antibody responses as a result of a natural exposure to S. pneumoniae.

The antibody responses of other streptococcal bacteria can be tested using the same method. On the basis of the results of this example, it is anticipated that streptococcal bacteria having a lytC deletion or reduced LytC function will have improved antibody responses compared to parental strain.

### Example Six: Enhanced Capacity of Gamma-PN3 Vaccination to Elicit Antibodies that Bind to Encapsulated S. pneumoniae Serotypes

To determine whether Gamma-PN3 antibodies could bind to fully encapsulated S. pneumoniae isolates, IgG binding to whole pneumococci was assessed by flow cytometry.

To generate antibodies for analysis, mice were vaccinated I.M. with Gamma-PN2 and Gamma-PN3 (50 g total protein in 50 μL PBS with 20% (w/v) glycerol per mouse) as described herein. Control mice were mock vaccinated with PBS with 20% (w/v) glycerol only. Mice were immunised three times, two weeks apart. Serum was taken from all mice two weeks after the final immunisation.

### Detection of Gamma-PN3 and Gamma-PN2 Induced IgG Binding to Encapsulated Streptococcal Bacteria by Flow Cytometry

Sera from each group was pooled (n=5) and tested for IgG binding to the encapsulated S. pneumoniae serotype 2 strain D39 and encapsulated S. pneumoniae serotype 6A strain P9 by flow cytometry using a standard protocol. Briefly, S. pneumoniae D39 or 6A strains were cultured using a standard protocol. 1×10¹ colony forming units (CFU) of live bacteria from D39 or P9 strains were incubated with pooled mouse sera at a 1:50 dilution in PBS-BSA. Bacterial cells were centrifuged and washed twice, and primary IgG bound to the surface of the pneumococcal bacteria were detected by an anti-mouse IgG secondary antibody (FITC conjugated).

### Results and Discussion

Graphs in Figure Nine show the percentage of cells bound by IgG (D39 in panel A and P9 in panel C) and the geometric mean fluorescence intensity (GMFI; D39 in panel B and P9 in panel D) detected by flow cytometry after incubation of each serotype with pooled murine sera from the vaccine and control groups. Greater fluorescence intensity is indicative of more IgG binding to the surface of pneumococcal cells. Data were analysed by one-way ANOVA (** p<0.01, **** p<0.0001).

The results show that Gamma-PN3-induced antibodies can bind to multiple encapsulated pneumococcal serotypes, including encapsulated strains of different serotypes. Furthermore, the level of binding by Gamma-PN3-induced antibodies was significantly greater than that for Gamma-PN2-induced antibodies for both serotypes. This indicates that the deletion of the lytC gene enhances the antibody response to antigens present on encapsulated bacteria, including a bacterium of a different serotype as compared to the parental strain of GPN-003. This result indicates that vaccination with Gamma-PN3 induces cross-serotype protection against encapsulated streptococcal bacteria.

The antibody responses of other streptococcal bacteria can be tested using the same method. On the basis of the results of this example, it is anticipated that streptococcal bacteria having a lytC deletion or reduced LytC function will have improved antibody responses compared to its parental strain, including an increase in the cross-protective antibody response against encapsulated bacteria.

### Example Seven: Induction of S. pneumoniae Gamma-PN3-Specific Serum Antibody Responses in Rabbits Intramuscularly Vaccinated with Gamma-PN3

To determine if Gamma-PN3 could induce serum antibody responses in rabbits, immunisation experiments were conducted.

### Vaccination of Rabbits

Gamma-PN2 and Gamma-PN3 were prepared as described herein. Outbred rabbits were intramuscularly (I.M.) vaccinated with Gamma-PN2 or Gamma-PN3 (500 μg total protein in 0.5 mL PBS per rabbit). Rabbits received three immunizations 3 weeks apart. Gamma-PN2 and Gamma-PN3 were administered in the absence of an adjuvant.

### Detection of IgG Responses Post-Vaccination

Serum was taken from all rabbits prior to immunizations (denoted as ‘pre-bleed’), and 3 weeks after the final immunization. Individual serum samples were tested for total IgG by direct ELISA using a standard protocol and using Gamma-PN3 as the coating antigen. Data were analysed by one-way ANOVA (nd=not detected) (Figure Ten).

### Results and Discussion

Figure Ten shows IgG titres pre-immunisation and post-third immunisation for each individual rabbit (panel A), and as mean IgG titres (±S.E.M.) (panel B) within each vaccine group. Vaccination with both Gamma-PN2 and Gamma-PN3 resulted in high titre antibody responses to the unencapsulated S. pneumoniae strain in rabbits following I.M. immunisation.

The ability of other streptococcal bacteria to induce specific serum antibodies can be tested using the same method as detailed above. On the basis of the results of this example, it is anticipated that immunisation of rabbits with other streptococcal bacteria having a lytC deletion or reduced LytC function would also increase the antibody response raised against the vaccine strain.

### Example Eight: Binding of Gamma-PN2 and Gamma-PN3 Induced Antibodies to Encapsulated S. pneumoniae

To further investigate the binding of Gamma-PN2- and Gamma-PN3-induced antibodies to fully encapsulated S. pneumoniae, flow cytometry analysis was performed.

Vaccination of Rabbits with GPN-002 and GPN-003

To generate antibodies for this analysis, outbred rabbits were intramuscularly (I.M.) vaccinated with Gamma-PN2 or Gamma-PN3 (500 μg total protein in 0.5 mL PBS per rabbit). Rabbits received three immunizations 3 weeks apart. Serum was taken from all rabbits prior to immunizations (denoted as ‘pre-bleed’), and 3 weeks after the final immunization.

### Detection of Gamma-PN3 and Gamma-PN2 Induced IgG Binding to Encapsulated Streptococcal Bacteria by Flow Cytometry

Individual serum samples were tested for IgG binding to the encapsulated S. pneumoniae serotype 7C using flow cytometry. Briefly, 1×10⁶ CFU of S. pneumoniae serotype 7C was incubated with immunised rabbit serum at a 1:200 dilution in PBS-BSA. Bacterial cells were pelleted and washed twice, and any primary IgG bound to the pneumococcal surface was then detected with an anti-rabbit IgG secondary antibody (FITC-conjugated).

### Results and Discussion

Flow cytometry detected the binding of IgG induced by vaccination with either GPN-002 or GPN-003 to S. pneumoniae serotype 7C, as shown in Figure Eleven:: (A) shows the relative fluorescence, (B) the percentage of serotype 7C cells that are bound by rabbit IgG antibodies, and (C) the mean fluorescent intensity of IgG antibodies bound to serotype 7C cells, indicative of the magnitude of IgG binding (panel C). Data were analysed by one-way ANOVA (* p<0.05, ** p<0.01, *** p<0.001, **** p<0.0001, ns not significant).

These results show that Gamma-PN2 and Gamma-PN3-induced antibodies are able to bind to encapsulated S. pneumoniae of a different serotype to the parental vaccine strain with high affinity. Furthermore, the level of binding of IgG to encapsulated S. pneumoniae of a different serotype was higher for Gamma-PN3 vaccinated animals than for Gamma-PN2 vaccinated animals. This indicates that the Gamma-PN3 vaccine induces more antibodies capable of binding to encapsulated bacteria of a different serotype than Gamma-PN2. Accordingly, the deletion of the lytC gene increased the antibody response. This indicates that GPN-003 induces cross-protective immunity, including against encapsulated strains.

The ability of other streptococcal bacteria to induce antibodies that bind to encapsulated bacteria can be tested using the same method as detailed above. On the basis of the results of this example, it is anticipated that immunisation of rabbits with other streptococcal bacteria having a lytC deletion or reduced LytC function would also increase the cross-protective antibody response against capsulated bacteria.

### Example Nine: Analysis of Gamma-PN2- and Gamma-PN3-Induced Antibody Functionality in Mediating Opsonophagocytic Killing of Encapsulated S. pneumoniae

Host protection against pneumococcal infections is believed to be associated with opsonin-dependent phagocytosis. Therefore, in vitro opsonophagocytic killing activity (OPA) of pneumococcal-specific antibodies of functional antibody activity was investigated following vaccination of rabbits with Gamma-PN2 or Gamma-PN3.

Rabbits were intramuscularly (I.M.) vaccinated with Gamma-PN2 or Gamma-PN3 (500 μg total protein in 0.5 mL PBS per rabbit). Rabbits received three immunizations 3 weeks apart. Serum was taken from all rabbits prior to immunizations (denoted as ‘pre-bleed’), and 3 weeks after the final immunization. Individual or pooled serum samples were tested for opsonophagocytic killing activity against a panel of S. pneumoniae serotypes (serotypes 6A, 6B, 9N, 23A, 24F and 33F), using the opsonophagocytic killing assay protocol established by Nahm and Burton, 2014. Titres were determined as the reciprocal of the highest dilution of rabbit serum giving 50% or more killing of the input CFU. Where no killing was detected with neat serum (1:4 dilution when mixed with other assay components), this is denoted as a titre of s 4.

### Results and Discussion

Table Four presents opsonophagocytic indexes (OI) titres.

TABLE FOUR
Opsonophagocytic indexes (OI) titres of rabbit serum following
vaccination with Gamma-PN2 or Gamma-PN3 compared to pre-bleed
serum against various S. pneumoniae serotypes

| | Pre-Bleed | Gamma-PN2 | Gamma-PN3 |
|---|---|---|---|
| Serotype | OI | OI | OI |
| 6A | ≤4 | 484 | 3027 |
| 6B | ≤4 | ≤4 | 36 |
| 9N | 65 | 201 | 246 |
| 23A | ≤4 | 1370 | 2077 |
| 24F | 6 | 18,754 | 24,877 |
| 33F | ≤4 | 4,225 | 5,479 |

The results show that Gamma-PN3 induced higher opsonophagocytic killing activity than Gamma-PN2 against all of the tested S. pneumoniae serotypes. This indicates that the deletion of the lytC gene enhances the antibody response to opsonise and kill encapsulated pneumococci. This result also indicates that Gamma-PN3 induces cross-protective responses against different S. pneumoniae serotypes. This ability of Gamma-PN3 to induce cross-protective responses against further S. pneumoniae serotypes or other streptococcal bacterial species can be tested using the same method as described herein.

Further, the ability of other streptococcal bacteria to induce antibodies that opsonise a range of streptococcal bacteria can be tested using the method as detailed above. On the basis of the results of this example, it is anticipated that immunisation of rabbits with other streptococcal bacteria having a lytC deletion or reduced LytC function would also increase the opsonisation response against a range of streptococcal bacteria.

### Example Ten: The Impact of lytC Mutation on the Transcription of the Conserved Antigenic Lipoprotein prsA

To further explore the improvement in immunogenicity exhibited by the vaccine strain containing a lytC deletion, the expression of antigenic genes in GPN-003 strain was assessed. Quantitative reverse transcription-PCR (qRT-PCR) was performed to assess whether a mutation in lytC induced changes in the expression of the conserved lipoprotein prsA. The prsA gene encodes a conserved lipoprotein that has been linked to serotype-independent protection.

### Quantitative Reverse Transcription-PCR

GPN-002 and GPN-003 strains were grown to equivalent optimal densities (OD₆₀₀ 0.6) in soytone medium (supplemented with 2.14 M MnSO₄). Culture samples were immediately transferred to RNAProtect Bacteria Reagent (Qiagen) and incubated for 5 minutes at room temperature. The cellular material was centrifuged, supernatant was removed, and bacterial pellets were stored at −80° C. Samples were enzymatically lysed with lysozyme and mutanolysin, and the RNA was extracted using the RNeasy Mini Kit (Qiagen) with an on-column DNasel treatment (Qiagen). The total RNA was quantified using Nanodrop spectrophotometer and diluted to 2 ng/L. The qRT-PCR was performed using SYBR q-PCR master mix and the QuantStudio 7 Flex Real-Time PCR System (Thermofisher Scientific). Primers for the amplification of prsA were designed using the UGENE extension Primer3 prsA_F (SEQ ID NO: 20) and prsA_R (SEQ ID NO: 21). The levels of gene transcription were normalised to the constitutively expressed gyrase A gene (gyrA) using the primers gyrA_F (SEQ ID NO: 22) and gyrA_R (SEQ ID NO: 23). Data is presented as mean prsA transcription relative to gyrA expression.

### Results and Discussion

As shown in Figure Twelve, comparisons of the gene expression of prsA showed higher expression in GPN-003 relative to GPN-002. These data suggest that a mutation in lytC resulted in enhanced gene expression of a key immunogen in vitro.

Further, the level of prsA expression in other streptococcal bacteria can be tested using the method as detailed above. On the basis of the results of this example, it is anticipated that other streptococcal bacteria having a lytC deletion or reduced LytC function would also have increased expression of prsA.

### Example Eleven: GPN-002 and GPN-003 are Avirulent in Mice

To explore the possibility of GPN-003 being used as an attenuated vaccine, the virulence of GPN-002 and GPN-003 was determined in mice.

Female Swiss mice (5 per group) were inoculated intraperitoneally with a lethal dose of S. pneumoniae serotype 2 (strain D39, 10⁴ CFU/mouse) or GPN-002 or GPN-003 at a 10,000-fold higher dose (10⁸ CFU/mouse). Mice were monitored for overall survival and weighed daily up to 7 days.

### Results and Discussion

As shown in Figure Thirteen (A), all mice inoculated with the lethal dose of D39 reached a moribund state within 24 hours. Conversely, mice receiving a far higher dose of GPN-002 or GPN-003 exhibited no clinical symptoms of infection and experienced no weight loss (Figure Thirteen (B)).

These data provide evidence that GPN-002 and GPN-003 are avirulent and could be used as attenuated vaccines.

### Prophetic Example One: Construction of GPN-001 Strain Harbouring Unmarked Deletion of lytC

The antibiotic resistance cassette can be removed from GPN001ΔlytC::kanR to generate GPN001ΔlytC. Removal of the antibiotic resistance gene produces a vaccine strain more suitable for human use.

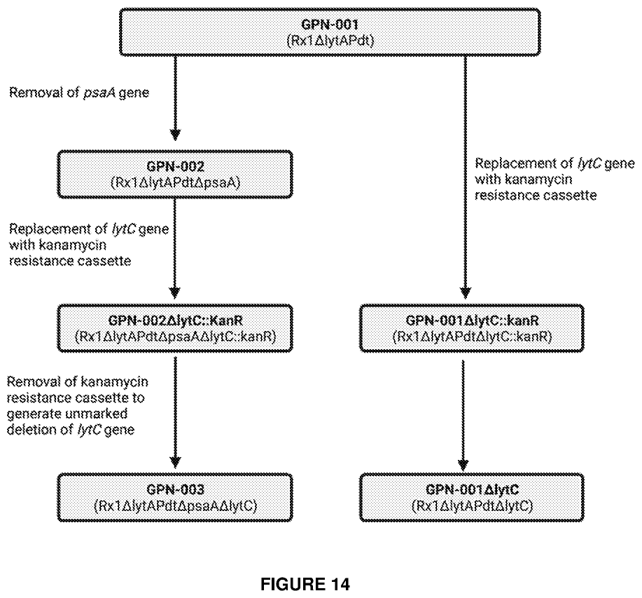

The kanamycin resistance cassette introduced into GPN-001 in strain GPN-001ΔlytC::kanR described earlier herein will be removed to generate an unmarked deletion of the lytC coding region as depicted in FIG. 14. Oligonucleotide primers were designed to PCR amplify regions of approximately 2 kb directly upstream of the lytC gene using primers lytC_F1 (SEQ ID NO: 2) and lytC_MD_R (SEQ ID NO: 8) and downstream of the lytC gene using primers lytC_MD_F (SEQ ID NO: 9); and lytC_R2 (SEQ ID NO: 5), to incorporate approximately 20-30 base pairs (bp) of complementary homology to the regions immediately upstream and downstream of the lytC region. The linear fragment of these two products will be generated either by overlap extension PCR using primers lytC_F1 (SEQ ID NO: 2 and lytC_R2 (SEQ ID NO: 5).

Alternatively, a linear fragment could be amplified directly from the GPN-003 strain by PCR. The linear markerless deletion fragment (lytC-) will be transformed into the GPN-001ΔlytC::kanR resistant strain essentially as described above, except with an enrichment step following the addition of the linear DNA to facilitate selection of kanamycin-sensitive clones. Briefly, S. pneumoniae GPN-001ΔlytC competent cells will be incubated with 100 ng/mL competence stimulating peptide 1 (CSP-1) for 15 minutes. The linear lytC-fragment will be added to the transformation reaction and enrichment of the deletion strain will be achieved by culturing in the presence of kanamycin, followed by ampicillin treatment. Successful incorporation will be determined by assessment of antibiotic resistance profile by patch testing on kanamycin-, and un-supplemented blood agar plates, sequencing, and size analysis by agarose gel electrophoresis. The resulting strain, S. pneumoniae GPN-001ΔlytC-, will harbour an unmarked lytC deletion suitable for clinical use.

All genetic modifications introduced in GPN-001ΔlytC relative to Rx1 will be confirmed based on size using the following oligonucleotide primers:
lytA: lytA_seq_F (SEQ ID NO: 12) and lytA_seq_R (SEQ ID NO: 13);
lytC: lytC_seq_F (SEQ ID NO: 14) and lytC_seq_R (SEQ ID NO: 15); and
pdt: PdT_seq_F (SEQ ID NO: 16) and PdT_seq_R (SEQ ID NO: 17).

The sequence of the lytC deletion in GPN-001ΔlytC will be further confirmed by Sanger Sequencing. The sequence of the 3′ end of the pdt region of GPN-001ΔlytC will also be confirmed by Sanger Sequencing.

### Prophetic Example Two: Analysis of Total IgG and Subclass IgG Responses from Mice Vaccinated with Gamma-PN1 and Gamma-PN1ΔlytC

To assess whether a vaccine developed based on the GPN-001ΔlytC strain induced altered antibody responses compared with GPN-001, a vaccination experiment in an animal model will be conducted. The GPN-001 and GPN-001ΔlytC strains will be inactivated by exposure to gamma radiation as described herein and the resulting vaccines are designated Gamma-PN1 and Gamma-PN1ΔlytC, respectively. The capacity of these vaccines to induce pneumococcal-specific antibody responses and the effect on IgG subclasses will be investigated.

Outbred Swiss mice will be intramuscularly (I.M) vaccinated with 50 g total protein of Gamma-PN1 and Gamma-PN1ΔlytC three times, two weeks apart. Control mice will be mock vaccinated with PBS with 20% (w/v) glycerol. Two weeks post final vaccination, serum will be taken from all mice. Serum samples will be tested for the S. pneumoniae-specific total IgG responses by direct Enzyme Linked Immunosorbent Assay (ELISA). Rx1, the un-encapsulated derivative of D39 (serotype 2) will be used as the coating antigen. The IgG subclass IgG1, IgG2a, IgG2b, and IgG3 responses will be tested by ELISA using Rx1, the un-encapsulated derivative of D39 (serotype 2) as the coating antigen.

### Prophetic Example Three: Capacity of Antibodies Induced by Gamma-PN1 and Gamma-PN1ΔlytC to Bind Encapsulated S. pneumoniae Serotypes

To generate antibodies for analysis, mice will be vaccinated L.M with Gamma-PN1 and GPN-001ΔlytC (50 g total protein in 50 L PBS/glycerol per mouse). Control mice will be mock-vaccinated with PBS/glycerol only. Mice will be immunised three times, two weeks apart. Serum will be taken from all mice two weeks after the final immunisation. Sera from each group will be pooled (n=5) and tested for IgG binding to the encapsulated serotypes 2 (strain D39) and 6A (strain P9) using flow cytometry. Briefly, 1×10¹ colony forming units (CFU) of live bacteria from each serotype will be incubated with pooled mouse sera at a 1:50 dilution in PBS-BSA. Bacterial cells will be centrifuged and washed twice, and primary IgG bound to the surface of the pneumococcal surface detected by an anti-mouse IgG secondary antibody (FITC conjugated). These data will show the percentage of cells bound by IgG and the geometric mean fluorescent intensity detected by flow cytometry after incubation of each serotype with pooled murine sera from the vaccine and control groups. Greater fluorescence intensity is indicative of more IgG binding to the surface of pneumococcal cells.

### Prophetic Example Four: Induction of S. pneumoniae Specific Serum Antibody Responses in Rabbits after Immunisation with Vaccines Harbouring Mutations in lytC

An immunisation experiment will be conducted to determine whether Gamma-PN1, Gamma-PN2, Gamma-PN3, Gamma-PN1ΔlytC::KanR or Gamma-PN1ΔlytC induce higher serum antibody responses in rabbits compared with vaccines based on the parental strains.

Outbred rabbits will be I.M. vaccinated with Gamma-PN1, Gamma-PN1ΔlytC::kanR, Gamma-PN1ΔlytC, Gamma-PN2, or Gamma-PN3 (500 g total protein in 0.5 mL PBS per rabbit). Rabbits will receive three immunisations 3 weeks apart, where serum will be taken prior to the first immunisation (termed ‘pre-bleed’) and 3 weeks after the final immunisation. Individual serum samples will be tested for total IgG by direct ELISA, using whole-cell Rx1 as the coating antigen.

### Prophetic Example Five: Binding of Gamma-PN1, Gamma-PN1ΔlytC::kanR, Gamma-PN1ΔlytC, Gamma-PN2, or Gamma-PN3-Induced Antibodies to Encapsulated S. pneumoniae

To assess the binding of Gamma-PN1, Gamma-PN1ΔlytC::kanR, Gamma-PN1ΔlytC, Gamma-PN2, or Gamma-PN3-induced antibodies (which target pneumococcal proteins) to fully encapsulated S. pneumoniae, flow cytometry analysis will be performed.

To generate antibodies for this analysis, outbred rabbits will be intramuscularly (I.M.) vaccinated with Gamma-PN1, Gamma-PN1ΔlytC::kanR, Gamma-PN1ΔlytC, Gamma-PN2, or Gamma-PN3 (500 μg total protein in 0.5 mL PBS per rabbit). Rabbits will receive three immunisations 3 weeks apart. Serum will be collected from all rabbits prior to immunizations (denoted as ‘pre-bleed’), and 3 weeks after the final immunization. Individual serum samples will be tested for IgG binding to encapsulated S. pneumoniae of a serotype different to the parental strain using flow cytometry. Briefly, 1×10⁶ CFU of live encapsulated bacteria will be incubated with rabbit serum at a 1:200 dilution in PBS-BSA. Bacterial cells will be centrifuged and washed twice, and any primary IgG bound to the pneumococcal surface will then be detected with an anti-rabbit IgG secondary antibody (FITC-conjugated). The percentage of pneumococcal cells that are bound by rabbit IgG antibodies and the mean fluorescent intensity of IgG antibodies bound to pneumococcal cells, indicative of the magnitude of IgG binding will be shown.

Results will define the binding of serum antibodies induced by Gamma-PN1, Gamma-PN1ΔlytC, Gamma-PN1ΔlytC-, Gamma-PN2, or Gamma-PN3 vaccination to encapsulated S. pneumoniae.

### Prophetic Example Six: Analysis of Gamma-PN1, Gamma-PN1ΔlytC::kanR, Gamma-PN1ΔlytC, Gamma-PN2, or Gamma-PN3-Induced Antibody Opsonophagocytic Killing of Encapsulated S. pneumoniae

Host protection against pneumococcal infections is mainly mediated by opsonin-dependent phagocytosis. Therefore, opsonophagocytic killing activity (OPA) of pneumococcal-specific antibodies is considered a useful in vitro measure of functional antibody activity. OPA titres will be determined following vaccination of rabbits with Gamma-PN1, Gamma-PN1ΔlytC::kanR, Gamma-PN1ΔlytC, Gamma-PN2, or Gamma-PN3.

Rabbits will be intramuscularly (I.M.) vaccinated with Gamma-PN1, Gamma-PN1ΔlytC::kanR, Gamma-PN1ΔlytC, Gamma-PN2, or Gamma-PN3 (500 μg total protein in 0.5 mL PBS per rabbit). Rabbits will receive three immunizations 3 weeks apart. Serum will be taken from all rabbits prior to immunizations (denoted as ‘pre-bleed’), and 3 weeks after the final immunization. Individual or pooled serum samples will be tested for opsonophagocytic killing activity against a panel of S. pneumoniae serotypes, using the opsonophagocytic killing assay protocol established by Nahm and Burton (2014).

The opsonophagocytic indexes (OI) titres will be determined as the reciprocal of the highest 3-fold serial dilution of rabbit serum giving 50% or more killing of the input CFU. Where no killing is detected with neat serum (1:4 dilution when mixed with other assay components), the OI will be denoted as a titre of s 4.

Results will define the OPA responses of Gamma-PN1, Gamma-PN1ΔlytC::kanR, Gamma-PN1ΔlytC, Gamma-PN2, and/or Gamma-PN3 against different S. pneumoniae serotypes.

### Prophetic Example Seven: Impact of Deletion of lytC on the Antibody Profile Elicited by Vaccination

Experiments will be conducted to assess whether deletion of lytC alters the binding profile of serum IgG to a panel of known pneumococcal antigens.

Swiss mice will be vaccinated with 50 μg total protein of Gamma-PN1, Gamma-PN1ΔlytC::kanR, Gamma-PN1ΔlytC, Gamma-PN2, Gamma-PN3 three times at fortnightly intervals. Control mice will be mock vaccinated with PBS/glycerol. At 14 days post final vaccination, sera will be harvested and tested for binding of IgG to a panel of 289 pneumococcal antigenic proteins. Control mice will be used to calculate pneumococcal-specific antibody titres.

### Prophetic Example Eight: Analysis of Gene Expression in Strains Harbouring a lytC Deletion Relative to Parental Strains

Experiments will be conducted to assess any changes in gene transcription elicited by deletion of lytC.

S. pneumoniae vaccine strains GPN-001, GPN-001ΔlytC, GPN-002, GPN-002ΔlytC and/or GPN-003 will be cultured in standard animal-free soy-based medium until mid-logarithmic phase and the RNA will be isolated using conventional techniques (e.g., Qiagen RNeasy Mini Kit). Transcriptomic RNA-sequencing analysis will be performed essentially as previously described. Briefly, ribosomal RNA content will be depleted using established methods (e.g., Epicentre Bacterial Ribozero Kit, Illumina), and barcoded libraries will be generated (e.g., Ultra-Directional RNA kit, New England Biolabs). Prepared libraries will be sequenced (e.g., Illumina HiSeq2500) and reads will be aligned to the S. pneumoniae Rx1 (GenBank accession number CP079923) genome (e.g., BOWTIE2). Various tools will be used to derive the counts and differential gene expression for each gene (e.g., SAMtools, BEDtools, R, DESeq Library).

### Prophetic Example Nine: Proteomic Analysis of Strains Harbouring a lytC Deletion Relative to Parental Strains

Experiments will be conducted to assess any changes in protein abundance elicited by deletion of lytC.

S. pneumoniae vaccine strains GPN-001, GPN-001ΔlytC, GPN-002, GPN-002ΔlytC and/or GPN-003 will be cultured in standard animal-free soy-based medium, harvested by centrifugation, and lysed using a combination of chemical and mechanical methods. Comparative proteomic separation and protein identification will be performed using standard techniques (e.g., 2-dimensional differential gel electrophoresis [2D-DIGE] and mass spectrometry).

### Prophetic Example Ten: Construction of Vaccine Strain Harbouring Modifications in lytA, lytC, psaA and pdT in Alternative Unencapsulated Background Strains

Experiments to date have been performed based on derivatives of the S. pneumoniae Rx1 strain, an unencapsulated derivative of the serotype 2 strain D39. However, a similar outcome is expected for any unencapsulated strain.

Experiments will be conducted to genetically modify a strain to remove genes from the capsule biosynthetic locus (cps genes) or by culturing in a manner that leads to spontaneous loss of capsule genes. Capsule genes can be removed using conventional genetic methods or by standard in vitro serial passaging of any serotype, including but not limited to serotype 1, 2, 3, 4, 5, 6A, 6B, 6C, 6D, 7A, 7B, 7C, 7F, 8, 9A, 9L, 9N, 9V, 10A, 10B, IOC, 10F, 11A, 11B, 11C, 11D, 11F, 12A, 12B, 12F, 13, 14, 15A, 15B, 15C, 15F, 16A, 16F, 17A, 17F, 18A, 18B, 18C, 18F, 19A, 19B, 19C, 19F, 20, 21, 22A, 22F, 23A, 23B, 23F, 24A, 24B, 24C, 24F, 25A, 25F, 27, 28A, 28F, 29, 31, 32A, 32F, 33A, 33B, 33C, 33D, 33F, 34, 35A, 35B, 35C, 35F, 36A, 36B, 37, 38, 39, 40, 41A, 41F, 42, 43, 44, 45, 46, 47A, 47F, and/or 48.

For example, the genes of the cps locus, including but not limited to cpsA/wzg/capA, cpsB/wzh/capB, cpsC/wzd/capC, cpsD/wze/capD, cpsE/wchA/capE, cpsT, cpsF, cpsG, cpsH/wzy, cpsI, cpsJ/wzx, cpsK, cpsP, cpsL, cpsM, cpsN, cpsO, can be modified genetically by conventional methods to abrogate capsule production/expression. The lack of capsule or reduction in capsule will be confirmed by measurements of capsule content, serotyping assays and microscopy, and experiments will be conducted to introduce further modifications in one or more of the following genes: lytA, ply/pdt, psaA, and lytC by conventional methods of genetic manipulation.

As one non-limiting example, experiments will be performed to remove the cps4E gene from the well-characterised TIGR4 strain (serotype 4). Oligonucleotide primers will be designed to amplify a 2 kb region upstream and downstream of the cps4E gene and incorporate regions of complementarity to an antibiotic resistance cassette. A linear fragment of the 2 kb flanking regions encompassing an antibiotic resistance cassette will be generated by overlap extension PCR and confirmed by plating on antibiotic supplemented blood agar plates. Experiments will then be conducted to introduce further modifications in one or more of the following genes: lytA, ply/pdt, psaA, and lytC by conventional methods of genetic manipulation. Such resulting strain could be defined as TIGR4Δcps4EΔlytA PdTΔpsaAΔlytC, which is analogous to the GPN-003 strain described herein.

The resulting vaccine strains will be tested in animal models by immunisation of animals (e.g., mouse or rabbit) at several time points via the intranasal, intramuscular, subcutaneous, or intraperitoneal route as a live attenuated or inactivated vaccine (e.g., by chemical or physical methods such as exposure to high-energy photons). Serum will be collected two weeks after the final immunisation and assessed for immunological responses, for example, antibody titre (e.g., total IgG titre), antibody binding (e.g., IgG binding to encapsulated bacteria), antibody function (e.g., opsonophagocytic responses) and protective efficacy.

Data from these experiments will confirm the immunogenicity of vaccines generated based on alternative unencapsulated strains, such as S. pneumoniae TIGR4Δcps4EΔlytA PdTΔpsaAΔlytC.

### Prophetic Example Eleven: Construction of Vaccine Strains Harbouring Modifications in lytA, lytC, psaA and pdT in Encapsulated Background Strains Engineered to Express Lower Amount of Capsule on the Cell Surface

Experiments to date have been performed based on the S. pneumoniae Rx1, an unencapsulated derivative of the serotype 2 strain D39. However, a similar outcome is expected for strains that have been engineered to express lower levels of capsule that also permit exposure of key surface antigens. Experiments will be conducted to engineer encapsulated strains of bacteria to introduce genetic modifications that reduce, but do not abolish, capsular polysaccharide expression or alter its cell surface localisation or attachment. Strains from any serotype, including but not limited to serotype 1, 2, 3, 4, 5, 6A, 6B, 6C, 6D, 7A, 7B, 7C, 7F, 8, 9A, 9L, 9N, 9V, 10A, 10B, IOC, 10F, 11A, 11B, 11C, 11D, 11F, 12A, 12B, 12F, 13, 14, 15A, 15B, 15C, 15F, 16A, 16F, 17A, 17F, 18A, 18B, 18C, 18F, 19A, 19B, 19C, 19F, 20, 21, 22A, 22F, 23A, 23B, 23F, 24A, 24B, 24C, 24F, 25A, 25F, 27, 28A, 28F, 29, 31, 32A, 32F, 33A, 33B, 33C, 33D, 33F, 34, 35A, 35B, 35C, 35F, 36A, 36B, 37, 38, 39, 40, 41A, 41F, 42, 43, 44, 45, 46, 47A, 47F, and/or 48 can be engineered to reduce capsule levels on the cell surface.

One example of how this can be achieved is by modification of genes, such as Cps2A, LytR and Psr, that have been implicated in the retention of capsule at the cell wall. Alternatively, certain point mutations in cpsE have been shown to reduce total capsule production in clinical isolates. Mutations in vaccine strains to remove or alter these genes will be constructed using conventional methods, either singly or in combination, and are expected to result in a reduction in the amount of capsule. The reduction in cell-associated capsule will be confirmed by measurements of the capsule abundance. The capacity to further expose protein antigens on the surface and cell membrane will be assessed by comparing antibody binding to modified and the parental strains using serum raised in animals using unencapsulated vaccine strains (e.g., GPN-003), or using specific proteins that represent classes of proteins exposed to varying degrees on the surface (e.g., PspA, PcpA, PrtA, PhtE, PiuA, AdcA etc). Experiments will be conducted to generate encapsulated vaccine strains with these modifications by introducing further modifications in one or more of the following genes: lytA, ply/pdt, psaA, and lytC by conventional methods of genetic manipulation. For example, a vaccine strain could be made with a strain Streptococcus pneumoniae D39Δcps2KΔlytRΔlytAPdTΔpsaAΔlytC.

The resulting vaccines will be tested in animal models by immunisation of animals (e.g., mouse or rabbit) at several time points via the intranasal, intramuscular, subcutaneous, or intraperitoneal route as a live attenuated or inactivated vaccine (e.g., by chemical or physical methods such as exposure to high-energy photons). Serum will be collected two weeks after the final immunisation and assessed for immunological responses (e.g., antibody titre (total IgG titre), antibody binding (IgG binding to encapsulated bacteria), antibody function (opsonophagocytic responses) and protective efficacy.

Data from these experiments will confirm the immunogenicity of vaccines generated based on encapsulated strains that have been engineered for altered surface capsule abundance.

### Prophetic Example Twelve: Construction of Vaccine Strain Harbouring Modifications in lytA, lytC, psaA and pdT in Alternative Encapsulated Background Strains Cultured in a Manner that Reduces Capsule Expression

Experiments to date have been performed based on S. pneumoniae Rx1, an unencapsulated derivative of the serotype 2 strain D39. However, a similar outcome is expected for encapsulated strains that have been cultured in a manner that results in a lower expression of capsular polysaccharide that permits the exposure of key surface antigens. Strains from any serotype, including but not limited to serotype 1, 2, 3, 4, 5, 6A, 6B, 6C, 6D, 7A, 7B, 7C, 7F, 8, 9A, 9L, 9N, 9V, 10A, 10B, IOC, 10F, 11A, 11B, 11C, 11D, 11F, 12A, 12B, 12F, 13, 14, 15A, 15B, 15C, 15F, 16A, 16F, 17A, 17F, 18A, 18B, 18C, 18F, 19A, 19B, 19C, 19F, 20, 21, 22A, 22F, 23A, 23B, 23F, 24A, 24B, 24C, 24F, 25A, 25F, 27, 28A, 28F, 29, 31, 32A, 32F, 33A, 33B, 33C, 33D, 33F, 34, 35A, 35B, 35C, 35F, 36A, 36B, 37, 38, 39, 40, 41A, 41F, 42, 43, 44, 45, 46, 47A, 47F, and/or 48 can be cultured to reduce overall capsule expression.

Experiments will be conducted to generate encapsulated vaccine strains with these modifications by introducing modifications in one or more of the following genes: lytA, ply/pdt, psaA, and lytC by conventional methods of genetic manipulation. One approach to tune the expression of capsule is by overloading the bacteria with zinc. Zinc excess impacts the activity of the phosphoglucomutase enzyme, which is required for the production of key sugar precursors for capsular polysaccharide biosynthesis, resulting in less capsule. Various methods can be used to overload the bacteria with zinc, including culturing of the bacteria in medium supplemented with a molar excess of zinc ions (e.g., by supplementing the medium with ZnSO₄) relative to manganese ions, the engineering of strains to remove the zinc export protein gene (czcD), or through chemical treatments such as ionophores during growth (e.g., zinc pyrithione, PBT2 etc) that increase the translocation of zinc across biological membranes.

The resulting vaccines will be tested in animal models by immunisation of animals (e.g., mouse or rabbit) at several time points via the intranasal, intramuscular, subcutaneous, or intraperitoneal route as a live attenuated or inactivated vaccine (e.g., by chemical or physical methods such as exposure to high-energy photons). Serum will be collected two weeks after the final immunisation and assessed for immunological responses (e.g., antibody titre [e.g., Total IgG titre], antibody binding [e.g., IgG binding to encapsulated bacteria] and antibody function [opsonophagocytic responses]) and protective efficacy.

### REFERENCES

Corsini, B., Aguinagalde, L., Ruiz, S., Domenech, M., & Yuste, J. (2021). Vaccination With LytA, LytC, or Pce of Streptococcus Pneumoniae Protects Against Sepsis by Inducing IgGs That Activate the Complement System. Vaccines, 9(2), 186.
Croucher, N. J., Campo, J. J., Le, T. Q., Liang, X., Bentley, S. D., Hanage, W. P., & Lipsitch, M. (2017). Diverse evolutionary patterns of pneumococcal antigens identified by pangenome-wide immunological screening. Proceedings of the National Academy of Sciences, 114(3), E357-E366.
Eldholm, Vegard, et al. (2009) “Fratricide in Streptococcus pneumoniae: contributions and role of the cell wall hydrolases CbpD, LytA and LytC.” Microbiology 155.7: 2223-2234.
Holmlund, E., Quiambao, B., Ollgren, J., Jaakkola, T., Neyt, C., Poolman, J., . . . & Käyhty, H. (2009). Antibodies to pneumococcal proteins PhtD, CbpA, and LytC in Filipino pregnant women and their infants in relation to pneumococcal carriage. Clinical and Vaccine Immunology, 16(6), 916-923.
Nahm, M. H. and Burton, R. L. (2014) Protocol for multiplexed opsonophagocytic killing assay (UAB-MOPA) for antibodies against Streptococcus pneumoniae (Version E.02, December 2014). The Bacterial Respiratory Pathogen Reference Laboratory of the US NIH, WHO Reference Laboratory for Pneumococcal Serology, Departments of Pathology and Microbiology, University of Alabama at Birmingham Birmingham AL 35294-2170 USA https://www.vaccine.uab.edu/UAB-MOPA.pdf
Tomasz, A., & Waks, S. (1975). Enzyme replacement in a bacterium: phenotypic correction by the experimental introduction of the wild type enzyme into a live enzyme defective mutant pneumococcus. Biochemical and Biophysical Research Communications, 65(4), 1311-1319.
Wizemann, T. M., Heinrichs, J. H., Adamou, J. E., Erwin, A. L., Kunsch, C., Choi, G. H., . . . & Koenig, S. (2001). Use of a whole genome approach to identify vaccine molecules affording protection against Streptococcus pneumoniae infection. Infection and immunity, 69(3), 1593-1598.

1. An immunogenic composition comprising an attenuated and/or killed streptococcal bacterial strain comprising a modification that attenuates, reduces or prevents functional expression of LytC or a homologue thereof.

2. The immunogenic composition of claim 1, wherein the modification comprises a deletion of the lytC gene or a homologue thereof.

3. The immunogenic composition of claim 1 or 2, wherein the immunogenic composition is a cross-protective immunogenic composition.

4. The immunogenic composition of any one of claims 1 to 3, wherein the attenuated and/or killed streptococcal bacterial strain is derived from a parental strain lacking the modification, and the attenuated and/or killed streptococcal bacterial strain improves effectiveness of an immune response compared to the parental strain.

5. The immunogenic composition of any one of claims 1 to 4, wherein the attenuated and/or killed streptococcal bacterial strain is derived from a parental strain lacking the modification, and the attenuated and/or killed streptococcal bacterial strain has improved stability compared to the parent strain.

6. The immunogenic composition of any one of claims 1 to 5, wherein the attenuated and/or killed streptococcal bacterial strain further comprises modification or deletion of genes selected from the group consisting of lytA or a homologue thereof, ply or a homologue thereof, and psaA or a homologue thereof.

7. The immunogenic composition of any one of claims 1 to 6, wherein the attenuated and/or killed streptococcal bacterial strain further comprises a modification or deletion of a lytA gene of a homologue thereof.

8. The immunogenic composition of any one of claims 1 to 7, wherein the attenuated and/or killed streptococcal bacterial strain further comprises a modification or deletion of a ply gene or a homologue thereof.

9. The immunogenic composition of any one of claims 1 to 8, wherein the attenuated and/or killed streptococcal bacterial strain further comprises a toxoid version of ply designated PdT or a homologue thereof.

10. The immunogenic composition of any one of claims 1 to 9, wherein the attenuated and/or killed streptococcal bacterial strain further comprises a modification or deletion of a psaA gene or a homologue thereof.

11. The immunogenic composition of any one of claims 1 to 10, wherein the attenuated and/or killed streptococcal bacterial strain further comprises a modification or deletion of a lytA gene or a homologue thereof, a modification or deletion of a ply gene or a homologue thereof and a modification or deletion of a psaA gene or a homologue thereof, and further comprises a toxoid version of plydesignated PdT or a homologue thereof.

12. The immunogenic composition of any one of claims 1 to 11, wherein the attenuated and/or killed streptococcal bacterial strain expresses capsular polysaccharide.

13. The immunogenic composition of any one of claims 1 to 12, wherein the attenuated and/or killed streptococcal bacterial strain expresses a reduced level of capsular polysaccharide or no capsular polysaccharide.

14. The immunogenic composition of any one of claims 1 to 13, wherein the composition comprises a single attenuated and/or killed streptococcal bacterial strain comprising a modification that attenuates, reduces or prevents functional expression of LytC or a homologue thereof.

15. The immunogenic composition of any one of claims 1 to 13, wherein the composition comprises a plurality of attenuated and/or killed streptococcal bacterial strains each comprising a modification that attenuates, reduces or prevents functional expression of LytC or a homologue thereof.

16. The immunogenic composition of any one of claims 1 to 15, wherein the attenuated and/or killed streptococcal bacterial strain is of a species selected from the group consisting of Streptococcus agalactiae, Streptococcus bovis, Streptococcus canis, Streptococcus dysgalactiae, Streptococcus equi, Streptococcus equinus, Streptococcus equisimilis, Enterococcus faecalis, Enterococcus faecium, Streptococcus iniae, Streptococcus milleri, Streptococcus mutans, Streptococcus pneumoniae, Streptococcus pyogenes, Streptococcus salivarius, Streptococcus sanguinis, Streptococcus suis, and Streptococcus uberis.

17. The immunogenic composition of any one of claims 1 to 16, wherein the attenuated and/or killed streptococcal bacterial strain comprises a Streptococcus pneumoniae strain.

18. The immunogenic composition of claim 17, wherein the Streptococcus pneumoniae strain is derived from Rx1.

19. The immunogenic composition of any one of claims 1 to 18, wherein the attenuated and/or killed streptococcal bacterial strain induces an immune response to an at least one target streptococcal species and/or serotypes thereof.

20. The immunogenic composition of any one of claims 1 to 19, wherein the attenuated and/or killed streptococcal bacterial strain induces an immune response to a plurality of target streptococcal species and/or serotypes thereof.

21. The immunogenic composition of any one of claims 1 to 20, wherein the attenuated and/or killed streptococcal bacterial strain induces a cross-protective immune response to the at least one target streptococcal species and/or serotypes thereof.

22. The immunogenic composition of any one of claims 1 to 21, wherein the attenuated and/or killed streptococcal bacterial strain induces a cross-protective immune response to a plurality of target streptococcal species and/or serotypes thereof.

23. The immunogenic composition of any one of claims 1 to 20, wherein the attenuated and/or killed streptococcal bacterial strain is derived from the same strain, serotype or species as the at least one target streptococcal species and/or serotypes thereof.

24. The immunogenic composition of any one of claims 1 to 22, wherein the attenuated and/or killed streptococcal bacterial strain is derived from a different strain, serotype or species as the at least one target streptococcal species and/or serotypes thereof.

25. The immunogenic composition of any one of claims 1 to 24, wherein the at least one target streptococcal bacterial species and/or serotype thereof selected from the group consisting of: Streptococcus agalactiae, Streptococcus bovis, Streptococcus canis, Streptococcus dysgalactiae, Streptococcus equi, Streptococcus equinus, Streptococcus equisimilis, Enterococcus faecalis, Enterococcus faecium, Streptococcus iniae, Streptococcus milleri, Streptococcus mutans, Streptococcus pneumoniae, Streptococcus pyogenes, Streptococcus salivarius, Streptococcus sanguinis, Streptococcus suis and Streptococcus uberis.

26. The immunogenic composition of any one of claims 1 to 25, wherein the immunogenic composition is a killed whole cell immunogenic composition.

27. The immunogenic composition of any one of claims 1 to 26, wherein the killed whole cell immunogenic composition is selected from the group consisting of a chemical treatment killed whole cell immunogenic composition, a thermal treatment killed whole cell immunogenic composition, an irradiation killed whole cell immunogenic composition, a high hydrostatic pressure killed whole cell immunogenic composition, a pulsed electric field killed whole cell immunogenic composition, an ultrashort pulsed laser killed whole cell immunogenic composition, an ultrasound under pressure killed whole cell immunogenic composition, UV-irradiation, and a microbial inactivation killed whole cell immunogenic composition.

28. The immunogenic composition of any one of claims 1 to 27, wherein the killed whole cell immunogenic composition is a photon-irradiation killed whole cell immunogenic composition.

29. The immunogenic composition of any one of claims 1 to 28, wherein the killed whole cell immunogenic composition is a gamma radiation killed whole cell immunogenic composition.

30. The immunogenic composition of any one of claims 1 to 29, wherein the killed whole cell immunogenic composition is an X-ray radiation killed whole cell immunogenic composition.

31. The immunogenic composition of any one of claims 1 to 30, wherein the killed whole cell immunogenic composition is a gamma-irradiated and X-ray-irradiated killed whole cell immunogenic composition.

32. The immunogenic composition of any one of claims 1 to 25, wherein the immunogenic composition is an attenuated whole cell immunogenic composition.

33. The immunogenic composition of any one of claims 1 to 32, further comprising at least one of an adjuvant, a pharmaceutically acceptable excipient, and a pharmaceutically acceptable carrier.

34. The immunogenic composition of any one of claims 1 to 33, wherein the composition does not comprise an adjuvant.

35. A vaccine composition comprising the immunogenic composition of any one of claims 1 to 34.

36. Use of the immunogenic composition of any one of claims 1 to 34 or the vaccine composition of claim 35 as an immunogen.

37. Use of the immunogenic composition of any one of claims 1 to 34 or the vaccine composition of claim 35 as a vaccine.

38. A method of inducing an immune response in a subject to at least one target streptococcal species and/or serotype thereof, the method comprising administering a therapeutically effective amount of the immunogenic composition of any one of claims 1 to 34 or the vaccine composition of claim 35 to the subject.

39. A method of inducing a cross-protective immune response in a subject to at least one target streptococcal species and/or serotype thereof, the method comprising administering a therapeutically effective amount of the immunogenic composition of any one of claims 1 to 34 or the vaccine composition of claim 35 to the subject.

40. A method of preventing or reducing an infection in a subject by at least one target streptococcal species and/or serotype thereof, the method comprising administering to a subject a therapeutically effective amount of the immunogenic composition of any one of claims 1 to 34 or the vaccine composition of claim 35 to the subject.

41. A method of preventing or reducing a disease or condition in a subject caused by an at least one target streptococcal species and/or serotype thereof, the method comprising administering to a subject a therapeutically effective amount of the immunogenic composition of any one of claims 1 to 34 or the vaccine composition of claim 35 to the subject.

42. The method of any one of claims 38 to 42 wherein the attenuated and/or killed streptococcal bacterial strain is derived from the same strain, serotype or species as the at least one target streptococcal species and/or serotype thereof.

43. The method of any one of claims 38 to 42 wherein the attenuated and/or killed streptococcal bacterial strain is derived from a different strain, serotype or species as the at least one target streptococcal species and/or serotype thereof.

44. The method of any one of claims 38 to 42 wherein the at least one target streptococcal species and/or serotype thereof comprises a plurality of target streptococcal species and/or serotypes thereof.

45. The method of any one of claims 38 to 42 wherein the target streptococcal species and/or serotype thereof comprises a streptococcal bacterial species and/or serotype thereof selected from the group consisting of: Streptococcus agalactiae, Streptococcus bovis, Streptococcus canis, Streptococcus dysgalactiae, Streptococcus equi, Streptococcus equinus, Streptococcus equisimilis, Enterococcus faecalis, Enterococcus faecium, Streptococcus iniae, Streptococcus milleri, Streptococcus mutans, Streptococcus pneumoniae, Streptococcus pyogenes, Streptococcus salivarius, Streptococcus sanguinis, Streptococcus suis and Streptococcus uberis.

46. The method of any one of claims 38 to 42 wherein the immunogenic composition or the vaccine composition is administered to the subject by an administration route selected from the group consisting of intranasal, intravenous, intramuscular, subcutaneous, oral, intraocular, transmucosal, and transdermal administration.

47. The method of claim 41, wherein the disease or condition is selected from the group consisting of a respiratory tract infection, pneumonia, ear infection, earache, middle ear infection, otitis media, sinusitis, meningitis, conjunctivitis, bacteraemia, septicaemia, a joint infection, a bone infection, septic arthritis, osteomyelitis, a soft tissue infection, cellulitis, myositis, periorbital cellulitis, an abscess, peritonitis, a cardiac infection, endocarditis, and pericarditis.

48. A method of preparing the immunogenic composition of any one of claims 1 to 34, or the vaccine composition of claim 35, the method comprising:
(i) photon-irradiating a preparation of the streptococcal bacterial strain comprising a modification that attenuates, reduces or prevents functional expression of LytC or a homologue thereof to thereby kill or physically attenuate the bacteria to produce photon-irradiated streptococcal bacteria; and
(ii) combining the photon-irradiated streptococcal bacteria with a pharmaceutically acceptable excipient, diluent and/or carrier, and optionally, an adjuvant.

49. The method of claim 48, wherein the photon-irradiation comprises exposing the streptococcal bacterial strain to gamma-radiation.

50. The method of claim 49, wherein the photon-irradiation comprises exposing the streptococcal bacterial strain to X-radiation.

51. The method of any one of claims 48 to 50, wherein said photon-irradiating comprises exposing the streptococcal bacterial strain to gamma-radiation and X-radiation.

52. The method of any one of claims 48 to 51, wherein the photon irradiating comprises exposing the streptococcal bacterial strain to photon-radiation at energies of at least 0.01 MeV.


SEQUENCE LISTING (26 sequences)

SEQ ID NO: 1 - DNA, 1893 bp, Streptococcus pneumoniae
  source 1..1893: mol_type = genomic DNA
tttggtcaagaagtcgcagacaaagttcgtgttcaatacggtggttctgttaaacctgaaaatgttgcttcatacatggcttgcccagacgttgacggtgcccttgtaggtggtgcgtcacttgaagctgaaagcttcttggctttgcttgactttgtaaaataatcagtaagtagcaaaagctaggtggaacagcattcagatgtctgttacattttttataggagaggaaagattgaaaataaaaattggattagcaagtatctgtttactaggcttggcaactagtcatgtcgctgcaaatgaaactgaagtagcaaaaacttcgcaggatacaacgacagcttcaagtagttcagagcaaaatcagtcttctaataaaacgcaaacgagcgcagaagtacagactaatgctgctgcctactgggatggggattattatgtaaaggatgatggttctaaagctcaaagtgaatggatttttgacaactactataaggcttggttttatattaattcagatggtcgttactcgcagaatgaatggcatggaaattactacctgaaatcaggtggatatatggcccaaaacgagtggatctatgacagtaattacaagagttggttttatctcaagtcagatggggcttatgctcatcaagaatggcaattgattggaaataagtggtactacttcaagaagtggggttacatggctaaaagccaatggcaaggaagttatttcttgaatggtcaaggagctatgatacaaaatgaatggctctatgatccagcttattctgcttatttttatctaaaatccgatggaacttatgctaaccaagagtggcaaaaagtgggcggcaaatggtactatttcaagaagtggggctatatggctcggaatgagtggcaaggcaactactatttgactggaagtggtgccatggcgactgacgaagtgattatggatggtgctcgctatatctttgcggcctctggtgagctcaaagaaaagaaagatttgaatgtcggctgggttcacagagatggtaaacgctatttctttaataatagagaagaacaagtgggaaccgaacatgctaagaaaatcattgatattagtgagcacaatggtcgtatcaatgattggaaaaaggttattgatgagaacgaggtggatggtgtcattgttcgtctaggttatagcggtaaagaagacaaggaattggcgcataacattaaggagttaaaccgtctgggaattccttatggtgtctatctctatacctatgctgaaaatgagaccgatgctgagaatgacgctaaacagaccattgaacttataaagaaatacaatatgaacctgtcttaccctatctattatgatgttgagaactgggaatatgtaaataagagcaagagagctccaagtgatacagacacttgggttaaaatcatcaacaagtacatggacacgatgaagcaggcgggttatcaaaatgtatatgtctatagctatcgtagtttattacagacgcgtttaaaacacccagatattttaaaacatgtaaactgggtagcggcctatacgaatgctttagaatgggaaaacccttattattcaggagaaaaaggttggcaatatacctcttctgaatacatgaaaggaatccaagggcgcgtagatgtcagcgtttggtattaagcgatgatttgaaagagggatgtgatagtagcaccctctttttctttgttttatgatagttcatcctcgagtaaattcaagatcttgctcggaaatgaagcttatatagtagattgaatatagacaaataccttgtgattggtaaaacattttagaaattcatttacctttcctaatcgacttgg

SEQ ID NO: 2 - DNA, 21 bp, synthetic construct
  source 1..21: mol_type = other DNA
ctgggctttatctgcgctatg

SEQ ID NO: 3 - DNA, 47 bp, synthetic construct
  source 1..47: mol_type = other DNA
ccatacttttgtgcccgtctttcctctcctataaaaaatgtaacaga

SEQ ID NO: 4 - DNA, 49 bp, synthetic construct
  source 1..49: mol_type = other DNA
gaattgttttagtacctagaattcaccgcgatgatttgaaagagggatg

SEQ ID NO: 5 - DNA, 23 bp, synthetic construct
  source 1..23: mol_type = other DNA
acttcctcaaccaattcttctcg

SEQ ID NO: 6 - DNA, 18 bp, synthetic construct
  source 1..18: mol_type = other DNA
acgggcacaaaagtatgg

SEQ ID NO: 7 - DNA, 27 bp, synthetic construct
  source 1..27: mol_type = other DNA
ggtgaattctaggtactaaaacaattc

SEQ ID NO: 8 - DNA, 52 bp, synthetic construct
  source 1..52: mol_type = other DNA
catccctctttcaaatcatcgctctttcctctcctataaaaaatgtaacaga

SEQ ID NO: 9 - DNA, 52 bp, synthetic construct
  source 1..52: mol_type = other DNA
tctgttacattttttataggagaggaaagagcgatgatttgaaagagggatg

SEQ ID NO: 10 - DNA, 24 bp, synthetic construct
  source 1..24: mol_type = other DNA
gtgtcggaactatcctgattgtag

SEQ ID NO: 11 - DNA, 22 bp, synthetic construct
  source 1..22: mol_type = other DNA
gagttgagcagatgcctgtatc

SEQ ID NO: 12 - DNA, 26 bp, synthetic construct
  source 1..26: mol_type = other DNA
gtttggtgttgaaaatagtttaacag

SEQ ID NO: 13 - DNA, 19 bp, synthetic construct
  source 1..19: mol_type = other DNA
cgactcggtttacattgtc

SEQ ID NO: 14 - DNA, 21 bp, synthetic construct
  source 1..21: mol_type = other DNA
tttggtcaagaagtcgcagac

SEQ ID NO: 15 - DNA, 25 bp, synthetic construct
  source 1..25: mol_type = other DNA
ccaagtcgattaggaaaggtaaatg

SEQ ID NO: 16 - DNA, 23 bp, synthetic construct
  source 1..23: mol_type = other DNA
agattttggacaatacagaagtg

SEQ ID NO: 17 - DNA, 24 bp, synthetic construct
  source 1..24: mol_type = other DNA
cctgtttgatagactcattttaac

SEQ ID NO: 18 - DNA, 420 bp, Streptococcus pneumoniae
  source 1..420: mol_type = genomic DNA
tttggtcaagaagtcgcagacaaagttcgtgttcaatacggtggttctgttaaacctgaaaatgttgcttcatacatggcttgcccagacgttgacggtgcccttgtaggtggtgcgtcacttgaagctgaaagcttcttggctttgcttgactttgtaaaataatcagtaagtagcaaaagctaggtggaacagcattcagatgtctgttacattttttataggagaggaaagagcgatgatttgaaagagggatgtgatagtagcaccctctttttctttgttttatgatagttcatcctcgagtaaattcaagatcttgctcggaaatgaagcttatatagtagattgaatatagacaaataccttgtgattggtaaaacattttagaaattcatttacctttcctaatcgacttgg

SEQ ID NO: 19 - DNA, 478 bp, synthetic construct
  source 1..478: mol_type = other DNA
gattcaagaaggcagtcgctttacagcagatcatccaggcttgccgatttcctatacaacttcttttttacgtgacaatgtagttgcgacctttcaaaacagtacagactatgttgagactaaggttacagcttacagaaacggagatttactgctggatcatagtggtgcctatgttgcccaatattatattacttgggatgaattatcctataatcatcaaggtaaggaagtcttgactcctaaggcttgggacagaaatgggcaggatttgacggctcactttaccactagtattcctttaaaagggaatgttcgtaatctctctgtcaaaattagagagggtaccgggcttgccttcgaatggtggcgtacggtttatgaaaaaaccgatttgccactagtgcgtaagcggacgatttctatttggggaacaactctctatcctcaggtagaggataaggtagaaaatgactag

SEQ ID NO: 20 - DNA, 20 bp, synthetic construct
  source 1..20: mol_type = other DNA
cgtgagccaatcacttcaaa

SEQ ID NO: 21 - DNA, 20 bp, synthetic construct
  source 1..20: mol_type = other DNA
ctcaaaataatctgcaatta

SEQ ID NO: 22 - DNA, 20 bp, synthetic construct
  source 1..20: mol_type = other DNA
actggtatcgcggttgggat

SEQ ID NO: 23 - DNA, 20 bp, synthetic construct
  source 1..20: mol_type = other DNA
acctgatttccccatgacaa

SEQ ID NO: 24 - protein, 158 aa, Streptococcus pneumoniae
IQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 25 - protein, 158 aa, synthetic construct
IQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIREGTGLAFEWWRTVYEKTDLPLVRKRTISIWGTTLYPQVEDKVEND

SEQ ID NO: 26 - DNA, 478 bp, Streptococcus pneumoniae; identical to SEQ ID NO: 19
  source 1..478: mol_type = genomic DNA